(19) United States
(12) Patent Application Publication
(10) Pub. No.: US 2026/0207666 A1
(43) Pub. Date: Jul. 23, 2026

(54) CD38 AS INTEGRATION SITE FOR ENHANCED FUNCTION OF GENE-MODIFIED IMMUNE CELLS

(71) Applicant: RESEARCH INSTITUTE AT NATIONWIDE CHILDREN'S HOSPITAL, Columbus, OH (US)

(72) Inventors: Dean LEE, Canal Winchester, OH (US); Meisam NAEIMI KARAROUDI, Columbus, OH (US)

(21) Appl. No.: 19/137,150

(22) PCT Filed: Dec. 11, 2023
(86) PCT No.: PCT/US2023/083400

§ 371 Date: Jun. 9, 2025

Related U.S. Application Data
(60) Provisional application No. 63/431,509, filed on Dec. 9, 2022.

(51) Int. Cl. A61K 35/17; A61K 40/15; A61K 40/31; A61K 40/42; C07K 14/705; C07K 16/28; C12N 5/0783; C12N 9/22; C12N 15/113

(52) U.S. Cl. CPC A61K 35/17; A61K 40/15; A61K 40/31; A61K 40/4222; A61K 40/4224; C07K 14/7051; C07K 14/70521; C07K 16/2896; C12N 5/0646; C12N 9/222; C12N 15/113; A61K 2239/48; C07K 2317/622; C12N 2310/20; C12N 2510/00

(57) ABSTRACT

Disclosed herein is a genetically engineered cells expressing a chimeric antigen receptor and methods and uses thereof.

### REFERENCE TO CROSS-RELATED APPLICATION

This PCT application claims priority to, and the benefit of, U.S. Provisional Patent Application No. 63/431,509, filed Dec. 9, 2022, which is incorporated by reference herein in its entirety.

### REFERENCE TO SEQUENCE LISTING

The sequence listing submitted on Dec. 11, 2023, as an .XML file entitled “10935-023WO1_ST26.xml” created on Dec. 7, 2023, and having a file size of 87,749 bytes is hereby incorporated by reference pursuant to 37 C.F.R. § 1.52(e)(5).

### BACKGROUND

Modifying immune cells, such as NK cells and T cells, with a chimeric antigen receptor (CAR) can improve their targeting and increase specificity. However, genetic modifications for enhancing the function of immune cells with CAR remain to be explored. What are needed are new methods and vectors for engineering immune cells.

### SUMMARY

The present disclosure relates to a genetically engineered cell (including, for example, a T cell, B cell, NK cell, or NK T cell) and methods for the manufacture and use thereof.

In one aspect, disclosed herein is an engineered cells comprising a nucleic acid sequence encoding chimeric antigen receptor (CAR) (such as, for example, a CAR comprising the amino acid sequence as set forth in SEQ ID NO: 18, SEQ ID NO: 25, SEQ ID NO: 27, SEQ ID NO: 29, or SEQ ID NO: 31) comprising a single-chain variable fragment (scFV) that specifically binds to a target molecule (such as, for example, CD33 or CD38), wherein the nucleic acid sequence encoding the CAR is integrated into a CD38 gene locus. In some embodiments, the integration of the CD38 gene locus is at exon 1, exon 2, exon 3, exon 4, exon 5, exon 6, exon 7, and/or exon 8 of a CD38 gene.

Also disclosed herein are engineered cells of any preceding aspect, wherein the engineered cell has a decreased expression of CD38 as compared with a reference control.

In one aspect, disclosed herein are engineered cells of any preceding aspect, wherein the nucleic acid sequence encoding the CAR is integrated into the CD38 locus using a method comprising introducing into the cell a CRISPR/Cas endonuclease system with a CRISPR/Cas guide RNA, wherein the guide RNA targets the CD38 gene or a fragment thereof. In some aspects, the guide RNA comprises the sequence of CTGAACTCGCAGTTGGCCAT (SEQ ID NO: 1) or a fragment thereof.

Also disclosed herein are engineered cells of any preceding aspect, wherein the target molecule is a protein (including, but not limited to a mutated protein, a polysaccharide, or a toxin. In some aspects, the target molecule can be on a cell (such as, for example a normal cell or a tumor cell). In some aspects, the protein has an increased level in a subject or a cell in comparison to a reference control.

In one aspect, disclosed herein are engineered cells of any preceding aspect, wherein the CAR further comprises a transmembrane domain (such as, for example, a CD28 transmembrane domain, a CD3ζ transmembrane domain, or an NKG2D transmembrane domain) and/or a co-stimulatory domain (such as, for example, a 2B4 domain, a CD28 co-stimulatory domain, a 4-1BB co-stimulatory domain, or any combination thereof).

Also disclosed herein are pharmaceutical compositions comprising the engineered cell of any preceding aspect. In one aspect, the pharmaceutical composition can comprise an anti-CD38 inhibitor. In some aspects, the anti-CD38 inhibitor is an anti-CD38 antibody including, but not limited to daratumumab, isatuximab, MOR202, or TAK-079. In some aspects, the anti-CD38 antibody comprises an Fc domain or does not comprise an Fc domain.

In one aspect, disclosed herein are pharmaceutical compositions of any preceding aspect, further comprising an agent to increase an expression of CD38 (such as, for example all-trans retinoic acid (ATRA)).

Also disclosed herein are methods of making the engineered cell comprising a CAR of any preceding aspect the method comprising: a) obtaining a ribonucleoprotein (RNP) complex comprising a class 2 CRISPR/Cas endonuclease (Cas9) complexed with a corresponding CRISPR/Cas guide RNA and an Adeno-associated virus (AAV) vector (such as a serotype 6 (AAV6) vector) comprising a plasmid, nucleic acid, and/or construct comprising a polynucleotide sequence encoding a chimeric antigen receptor (CAR) polypeptide; wherein the polynucleotide sequence is flanked by homology arms; and wherein the homology arms are 800 bp in length or less; and b) introducing the polynucleotide sequence encoding the CAR polypeptide and the RNP complex into a cell; wherein the polynucleotide sequence encoding the CAR polypeptide is introduced into the cell via infection with the AAV into the cell; wherein the RNP complex hybridizes to a target sequence within the genomic DNA of the cell and the cell's DNA repair enzymes insert the polynucleotide sequence encoding the CAR polypeptide into the host genome at the target sequence within the genomic DNA of the cell thereby creating the engineered cell. In some aspects, the plasmid, nucleic acid, or construct further comprises a murine leukemia virus-derived (MND) promoter.

In one aspect, disclosed herein are methods of making an engineered cell of any preceding aspect, wherein the left homology arm and right homology arm are the same length. In some embodiments, the homology arms are each 1000 bp in length or less (for example, 600 bp). In other embodiments, the left homology arm and right homology arm have different lengths. In some embodiments, the homology arms specifically hybridize to a CD38 locus.

Also disclosed herein are methods of making an engineered cell of any preceding aspect, wherein the vector is a single stranded AAV (ssAAV) or a self-complimentary AAV (scAAV).

In some embodiments, disclosed herein are methods of making the engineered cell comprising a CAR of any preceding aspect said methods comprising a) obtaining a ribonucleoprotein (RNP) complex comprising a class 2 CRISPR/Cas endonuclease (Cas9) complexed with a corresponding CRISPR/Cas guide RNA and an AAV vector comprising a plasmid, nucleic acid, or construct comprising a polynucleotide sequence encoding a chimeric antigen receptor (CAR); wherein the polynucleotide sequence is adjacent to one protospacer adjacent motifs (PAMs) and one polynucleotide sequence encoding crispr RNA (crRNA) or flanked by one or more PAMs and one or more polynucleotide sequences that encode crRNAs; and b) introducing the polynucleotide sequence encoding the CAR polypeptide and the RNP complex into a cell; wherein the plasmid, nucleic acid, or construct is introduced into the cell via infection with the Adeno-associated virus (AAV) into the cell; wherein in the ribonucleoprotein (RNP) complex hybridizes to a target sequence within the genomic DNA of the cell, and the cell's DNA repair enzymes insert the polynucleotide encoding the CAR into the host genome at the target sequence, thereby creating the engineered cell.

Also disclosed herein are methods of making an engineered cell of any preceding aspect, wherein the plasmid, nucleic acid, or construct comprises in order one PAM sequence and one polynucleotide sequence that encodes crRNA, the polynucleotide sequence encoding the CAR polypeptide, one polynucleotide sequence that encodes crRNA, and one PAM sequence. In some embodiments, the plasmid, nucleic acid, or construct comprises in order the polynucleotide sequence encoding the CAR polypeptide, one polynucleotide sequence that encodes crRNA, and one PAM sequence.

In one aspect, disclosed herein is a method of treating, inhibiting, decreasing, reducing, ameliorating, and/or preventing a disease (e.g., cancer including, but not limited to leukemia or lymphoma) in a subject comprising administering to the subject a therapeutically effective amount of the engineered cell or the pharmaceutical composition of any preceding aspect. In some embodiments, the method further comprises administering to the subject a therapeutically effective amount of an anti-CD38 inhibitor. In some embodiments, the anti-CD38 inhibitor is an anti-CD38 antibody. In some embodiments, the anti-CD38 antibody comprises an Fc domain or does not comprise an Fc domain. In some embodiments, the anti-CD38 antibody comprises daratumumab, isatuximab, MOR202, or TAK-079.

Also disclosed herein is a method of treating, inhibiting, decreasing, reducing, ameliorating, and/or preventing a disease of any preceding aspect, further comprising administering to the subject a therapeutically effective amount of an agent to increase an expression of CD38 (such as, for example, all-trans retinoic acid (ATRA)). In some aspects, the subject was previously resistant to anti-CD38 therapy.

Also disclosed herein are methods of treating, reducing, decreasing, inhibiting, ameliorating, and/or preventing a minimal residual disease (MRD) (e.g., cancer including, but not limited to leukemia or lymphoma) in a subject comprising administering to the subject a therapeutically effective amount of the engineered cell or the pharmaceutical composition of any preceding aspect. In some embodiments, the method further comprises administering to the subject a therapeutically effective amount of an anti-CD38 inhibitor. In some embodiments, the anti-CD38 inhibitor is an anti-CD38 antibody. In some embodiments, the anti-CD38 antibody comprises an Fc domain or does not comprise an Fc domain. In some embodiments, the anti-CD38 antibody comprises daratumumab, isatuximab, MOR202, or TAK-079.

Also disclosed herein is a method of treating, inhibiting, decreasing, reducing, ameliorating, and/or preventing a MRD of any preceding aspect, further comprising administering to the subject a therapeutically effective amount of an agent to increase an expression of CD38 (such as, for example, all-trans retinoic acid (ATRA)). In some aspects, the subject was previously resistant to anti-CD38 therapy.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIGS. 1A and 1B show overcoming the fratricide of NK cells in Daratumumab therapy for multiple myeloma by generating CRISPR-modified CD38-KO ex vivo-expanded NK cells.

FIG. 2 shows generation of genetically edited human primary NK cells using Cas9/RNP and AAV.

FIG. 3 shows design of CD33CAR-Gen2 and CD33CAR-Gen4v2.

FIG. 4 shows anti-AML activity of CD33CAR-NK.

FIG. 5 shows that CD33CAR NK cells have enhanced anti-AML activity.

FIG. 6 shows that CD38 expressing on AML cells. AML cell co-culture with Wt-NK or CD33 CAR-NK cells induces AML cell death as shown by viability assessment or in SPADE plots (colored for pRb expression indicative of viable cycling cell), green arrows indicate live AML cells while red arrows indicate dead/dying AML cells. CD33 CAR-NK cells demonstrate increased AML cells killing, surviving AML cells have reduced CD33 surface expression and increased CD38 expression.

FIG. 7 shows a schematic indicating generation of fratricide resistant CD38KO/CD33-CAR NK cells.

FIG. 8 shows fratricide resistant CD38KO/CD33-CAR NK cells to target residual AML.

FIG. 9 shows Isatuximab based CD38KO/CD38-CAR NK cells.

FIG. 10 shows CD38 expressing AML, T-ALL, Burkitt Lymphoma and MM were targeted by CD38KO/CD38 CAR NK and T cells+ATRA.

FIG. 11 shows the summarized data of NK cell persistence in NSG mice during treatment. The frequency of human NK cells in PB at day 7 and their absolute number in spleen and bone marrow at day 9 are shown (n=5, mean±SD).

FIG. 12 shows map of construct CD38CAR_V1_ISA-LHIG-41_ssAAV-BackBone_Kan.

FIG. 13 shows map of construct CD38CAR_V2_ISA-HLIG-41_ssAAV-BackBone_Kan.

FIG. 14 shows map of construct CD38CAR_V3_ISA-LHCD8-41_ssAAV-BackBone_Kan.

FIG. 15 shows map of construct CD38CAR_V4_ISA-HLCD8-41_ssAAV-BackBone_Kan.

FIG. 16 shows map of construct CD38CAR_V4_ISA-HLCD8-41_ssAAV-BackBone_KanAAAE46.

FIG. 17 shows map of construct CD38CAR_V5_mISA-LHIG-41_ssAAV-BackBone_Kan.

FIGS. 18A, 18B, 18C, 18D, and 18E show the generation of CRISPR engineered CD38^{KO}/CD38-CAR human primary NK cells using Cas9/RNP and AAV. FIG. 18A shows a schemata of steps for CRISPR/RNP knockout of the CD38 gene and directed insertion of a CD38-CAR encoding DNA delivered by AAV6 vector with homology arms for CD38 targeting site. FIG. 18B shows CD38 (PE) and CAR (APC) expression levels measured by flow cytometry for binding of CD38 antigen, seven days post-stimulation. Constructs contain a 41BB signaling domain, a CD8α transmembrane domain/hinge, a CD3ζ stimulatory domain and reversed orderings of light and heavy chain orientations. FIG. 18C shows the relative percentage and intensity of CD38-CAR expression (n=10; mean±standard deviation [SD]). FIG. 18D shows the fold expansion of wildtype and CD38-CAR NK cells over 12 days after activation with irradiated, modified mbIL21-K562 cells and IL-2 show no significant change from wildtype human NK cells (n=10, mean±SD). P values were calculated using a two-way ANOVA, P: 0.0332 (*), 0.0021 (**), 0.0002 (***), <0.0001 (****). FIG. 18E shows the cytotoxicity observed for V3 and V4 CD38^{KO}/CD38-CAR NK cells against high CD38 expressing MM (H929), BL (Raji), and AML (MV-11) (n=5, mean±SD). P values were calculated using a two-way ANOVA, P<0.05 (*), <0.01 (**), 0.001 (***), <0.0001 (****).

FIGS. 19A, 19B, 19C, and 19D show that CD38-CAR NK cells exhibit enhanced cytotoxic function and cytokine secretion. CD38-CAR NK cells were tested against CD38 expressing AML, MM, and T-cell malignancies collected from patients at baseline. FIG. 19A shows NK and CD38-CAR NK killing of acute myeloid leukemia (AML-1) (n=3, mean±SD). FIG. 19B shows NK and CD38-CAR NK killing of multiple myeloma patient samples (MM-1, MM-2) (n=1, mean±SD). FIG. 19C shows NK and CD38-CAR NK killing of T-cell malignancies (hepatosplenic T cell lymphoma (HSTCL) and T cell prolymphocytic leukemia (T-PLL-1, T-PLL-2)) (n=1, mean±SD). All cytotoxicity P values were calculated using a two-way ANOVA, P<0.05 (*), <0.01 (**), 0.001 (***), <0.0001 (****). FIG. 19E shows Bio-Plex Pro Human Cytokine Assay was performed on the supernatant of wildtype and CD38-CAR NK cells co-cultured with CD38+ malignancies (n=9; mean±SD). P values were calculated using a paired Student t test; *P, 0.05, **P, 0.01, ***P, 0.001, ns, not significant.

FIGS. 20A, 20B, and 20C show ATRA upregulates CD38 expression on tumor cells and can enhance antitumor activity. FIG. 20A shows CD38 cell surface expression as measured by flow cytometry across the hematologic malignancies MM, AML, BL, and T-ALL after treatment with 10 nM of ATRA for, 48-hour. FIG. 20B shows the mean fluorescence intensity (MFI) of CD38 expression on cell lines with and without ATRA treatment. FIG. 20C shows cytotoxicity assays performed by co-culturing wildtype and CD38-CAR NK cells against AML, MM, BL, and T-cell malignancies with and without 48 hour, 10 nM ATRA pretreatments. MM1S (n=4), H929 (n=4), AML-10 (n=4), MV4-11 (n=4), Raji (n=4), Daudi (n=4), T-ALL patient primary cells (n=3) (mean±SD). P values were calculated using a two-way ANOVA, P<0.05 (*), <0.01 (**), 0.001 (***), <0.0001 (****).

FIGS. 21A, 21B, 21C, 21D, 21E, 21F, 21G, 21H, and 21I show mass cytometry analysis shows combination ATRA and CD38-CAR NK cell treatment decreases live AML population. Mass cytometry analysis was performed after 24 hour primary AML cell lines (AML-1 and AML-2) were cultured with wildtype or CD38-CAR NK cells with and without 48 hr, 10 nM ATRA pre-treatment. Eight distinct conditions were analyzed: (21A) wildtype NK cells alone, (21B) CD38-CAR NK cells alone, (21C) AML cells alone, (21D) AML+wildtype NK cells, (21E) AML+CD38-CAR NK cells, (21F) AML cells with ATRA, (21G) AML+ATRA+wildtype NK cells, and (21H) AML+ATRA+CD38-CAR NK cells. FIG. 21I shows a heat map showing surface marker expression in live AML cells after control or treatment groups, graphed in log 2 scale.

FIGS. 22A, 22B, 22C, and 22D show CD38^{KO}/CD38-CAR T cells generated by Cas9/RNP and AAV6 show antitumor activity. FIG. 22A shows CD38 (PE) and CAR (APC) expression levels measured by flow cytometry. FIG. 22B shows the relative percentage of CD38-CAR expression (n=5; mean±SD). FIG. 22C shows 10-day fold expansion of generated CD38-CAR T cells with matched donors (n=4, mean±SD). P values were calculated using a two-way ANOVA, P: 0.0332 (*), 0.0021 (**), 0.0002 (***), <0.0001 (****). FIG. 22D shows the cytotoxicity of CD38^{KO}/CD38-CAR T cells toward H929, Raji, and MV4-11 cell lines (n=4, mean±SD). P values were calculated using a two-way ANOVA, P<0.05 (*), <0.01 (**), 0.001 (***), <0.0001 (****).

FIGS. 23A, 23B, 23C, 23D, and 23E show AAVS1KO/CD38-CAR NK cells avoid fratricide. FIG. 23A shows CD38 (PE) and CAR (APC) expression levels measured by flow cytometry for the CD38-CAR inserted into different loci. All CAR-NK cells were generated with matched donors. FIG. 23B shows the fold expansion of wildtype and CD38-CAR NK cells over 12 days (n=6; mean±SD), P values were calculated using a two-way ANOVA, P<0.05 (*), <0.01 (**), 0.001 (***), <0.0001 (****). FIG. 23C shows relative percentage of CD38-CAR expression (n=6; mean±SD). FIG. 23D shows reverse-transcription quantitative PCR was performed using CD38 primer probes to detect transcription of the CD38 gene in the wildtype and AAVS1KO/CD38-CAR NK cells. FIG. 23E shows CD38 expression measured by Flow cytometry using a polyclonal anti-CD38 antibody.

FIGS. 24A, 24B, 24C, 24D, and 24E show AAVS1KO/CD38-CAR NK cells display enhanced cytotoxicity and metabolism. CD38-CAR NK cells were tested against CD38 expressing AML, MM, and BL. FIG. 24A shows CD38-CAR NK cell killing of CD38 expressing cell lines (n=3, mean±SD). P values were calculated using a two-way ANOVA, P<0.05 (*), <0.01 (**), 0.001 (***), <0.0001 (****). FIG. 24B shows oxygen consumption rate (OCR) for CD38-CAR NK cells. FIG. 24C shows glycolytic capacity, measured by the extracellular acidification rate (ECAR), observed in CD38-CAR NK cells. FIG. 24D shows spare respiratory capacity, a measure of the cell's ability to produce ATP in response to stress, measured in CAR NK cells. FIG. 24E shows CAR NK cells maximal respiration rates compared to wildtype.

FIG. 25 shows flow cytometry gating strategy. Gating strategy used to measure the CD38 and CD38-CAR expression in NK and T cell. Cells were gated for viability and lymphocyte populations before measuring CD38 and CAR expression.

FIG. 26 shows primary cell line CD38 expression. CD38 surface expression was measured using flow cytometry for primary cell lines of AML, MM, and T-cell malignancies.

FIG. 27 shows CD38-CAR cytokine production. Bio-Plex Pro Human Cytokine Assay was performed on the supernatant of wildtype and CD38-CAR NK cells co-cultured with CD38+ malignancies (n=9; mean±SD). P values were calculated using a paired Student t test; *P, 0.05, **P, 0.01, ***P, 0.001, ns, not significant.

FIG. 28 shows CD38-CAR NK cells metabolic function. Basal respiration and glycolytic function of CD38^{KO} and AAVS1^{KO} CAR NK cells compared to wildtype NK cells.

### DETAILED DESCRIPTION

The following description of the disclosure is provided as an enabling teaching of the disclosure in its best, currently known embodiment. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Administration” or “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

A “control” is an alternative subject or sample used in an experiment for comparison purposes. A control can be “positive” or “negative.”

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A DNA sequence that “encodes” a particular RNA is a DNA nucleic acid sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature, and which has not been intentionally modified by a human in the laboratory is wild type (and naturally occurring).

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

“Optional” or “optionally” means that the subsequently described event or circumstance may or may not occur, and that the description includes instances where said event or circumstance occurs and instances where it does not.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g. promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “promoter” as used herein is defined as a DNA sequence recognized by the synthetic machinery of the cell, or introduced synthetic machinery, required to initiate the specific transcription of a polynucleotide sequence.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define an target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

A “chimeric antigen receptor” is an artificial cell receptor used for immunotherapy. CAR are protein receptors that have been engineered to give immune cells (e.g., T cells, NK cells, NKT cells, B cells, or macrophages) an enhanced ability to target a specific protein. CAR receptors are chimeric because the antigen binding and cell activating functions have been combined into a single receptor.

The terms “cell,” “cell line” and “cell culture” include progeny. It is also understood that all progeny may not be precisely identical in DNA content, due to deliberate or inadvertent mutations. Variant progeny that have the same function or biological property, as screened for in the originally transformed cell, are included. The “host cells” used in the present invention generally are prokaryotic or eukaryotic hosts.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating or impeding one or more causes of a disorder or condition. Treatments according to the disclosure may be applied preventively, prophylactically, palliatively, or remedially. Treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of a cancer.

As used herein, the term, “deletion”, also called gene deletion, deficiency, or deletion mutation, refers to part of a chromosome or a sequence of DNA being left out during DNA replication. Deletion, or gene deletions can cause any number of nucleotides to be deleted from a single base to an entire piece of chromosome.

“Effective amount” of an agent refers to a sufficient amount of an agent to provide a desired effect. The amount of agent that is “effective” will vary from subject to subject, depending on many factors such as the age and general condition of the subject, the particular agent, or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. In some embodiments, a desired therapeutic result is the control of cancer. In some embodiments, a desired therapeutic result is the control of metastasis. In some embodiments, a desired therapeutic result is the reduction of tumor size. In some embodiments, a desired therapeutic result is the prevention and/or treatment of relapse. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein, “transgene” refers to exogenous genetic material (e.g., one or more polynucleotides) that has been or can be artificially provided to a cell. The term can be used to refer to a “recombinant” polynucleotide encoding any of the herein disclosed polypeptides that are the subject of the present disclosure. The term “recombinant” refers to a sequence (e.g., polynucleotide or polypeptide sequence) which does not occur in the cell to be artificially provided with the sequence, or is linked to another polynucleotide in an arrangement which does not occur in the cell to be artificially provided with the sequence. It is understood that “artificial” refers to non-natural occurrence in the host cell and includes manipulation by man, machine, exogenous factors (e.g., enzymes, viruses, etc.), other non-natural manipulations, or combinations thereof. A transgene can comprise a gene operably linked to a promoter (e.g., an open reading frame), although is not limited thereto. Upon artificially providing a transgene to a cell, the transgene may integrate into the host cell chromosome, exist extrachromosomally, or exist in any combination thereof.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compositions and Methods of Genetically Modifying Cells

CD38 expression on NK cells can interference with the efficacy of antibodies targeting CD38. The examples herein show that CD38KO NK cell are resistant to CD38-mediated fratricide and effective to suppress tumors. Further, deletion of CD38 in NK cells can enhance their metabolic profile. Disclosed herein are immune cells (including, for example, T cells, B cells, macrophages, NK cells, or NK T cells) with CAR, wherein these cells are generated by integrating a nucleic acid sequence encoding CAR into a CD38 locus for improving immune cell anti-tumor activity.

Accordingly, in one aspect, disclosed herein is an engineered cell (including, for example, a T cell, B cell, macrophage, NK cell, or NK T cell) comprising a nucleic acid sequence encoding a chimeric antigen receptor (CAR) (such as, for example, a CAR comprising the amino acid sequence as set forth in SEQ ID NO: 18, SEQ ID NO: 25, SEQ ID NO: 27, SEQ ID NO: 29, or SEQ ID NO: 31) comprising a single-chain variable fragment (scFV) that specifically binds to a target molecule, wherein the nucleic acid sequence encoding the CAR is integrated into a CD38 gene locus.

In some embodiments, the nucleic acid sequence encoding the CAR is integrated into a CD38 gene locus at exon 1, exon 2, exon 3, exon 4, exon 5, exon 6, exon 7, and/or exon 8 of a CD38 gene. Integration of the nucleic acid sequence encoding the CAR can lead to a deletion or disruption of a CD38 gene or a fragment thereof (e.g., exon 1, exon 2, exon 3, exon 4, exon 5, exon 6, exon 7, and/or exon 8 of a CD38 gene). In some embodiments, the engineered cell has a decreased expression of CD38 as compared with a reference control.

The nucleic acid sequence encoding the CAR is integrated into the CD38 locus using a method comprising introducing into the cell a CRISPR/Cas endonuclease system with a CRISPR/Cas guide RNA, wherein the guide RNA targets the CD38 gene or a fragment thereof. In some embodiments, the guide RNA comprises the sequence of CTGAACTCGCAGTTGGCCAT (SEQ ID NO: 1) or a fragment thereof. In general, “CRISPR system” or “CRISPR integration system” refers collectively to transcripts and other elements involved in the expression of or directing the activity of CRISPR-associated “Cas” genes. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

In some embodiments, the target molecule of the CAR is a protein, a peptide, a polysaccharide, or a toxin. The target molecule can be on a cell (e.g., on a normal cell or a tumor cell). In some embodiments, the target molecule is on a tumor cell.

In some examples, the target molecule of the CAR is a protein. In some embodiments, the protein is a mutated protein. In some embodiments, the protein has an increased level in a subject or a cell in comparison to a reference control. In some embodiments, the protein is increased by 10% or more (e.g., increased by 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or at least about a 2-fold, or at least about a 3-fold, or at least about a 4-fold, or at least about a 5-fold or at least about a 10-fold increase, or any increase between 2-fold and 10-fold or greater) in a subject or cell in comparison to a reference control.

As used herein “chimeric antigen receptor” or “CAR” refers to a chimeric receptor that targets a cancer antigen and serves to bring the cell expressing the receptor to a cancer cell expressing the target antigen. Typically, the CAR comprises a molecule that recognizes peptides derived from the tumor antigen presented by major histocompatibility (MHC) molecules, or an antibody or fragment thereof (such as for example, a Fab′, scFv, Fv) expressed on the surface of the CAR cell that targets a cancer antigen. The receptor is fused to a signaling domain (such as, for example, the CD3ζ domain for T cells and NKG2C, NKp44, or CD3ζ domain for NK cells or NK T cells) via a linker. Tumor antigen targets are proteins that are produced by tumor cells that elicit an immune response, particularly B cell, NK cell, NK T cells, and T cell mediated immune responses. The selection of the antigen binding domain will depend on the particular type of cancer to be treated. Tumor antigens are well known in the art and include, for example, a glioma-associated antigen, carcinoembryonic antigen (CEA), EGFRvIII, IL-llRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin B1, lectin-reactive AFP, Fos-related antigen 1, ADRB3, thyroglobulin, EphA2, RAGE-1, RU1, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, IL13Ra2, CA 19-9, CA 72-4, CAM 17.1, NuMa, K-ras, beta-Catenin, CDK4, Mum-1, p 15, p 16, 43-9F, 5T4, 791Tgp72, alphafetoprotein, beta-HCG, BCA225, BTAA, CA 125, CA 15-3CA 27.29BCAA, CA 195, CA 242, CA-50, CAM43, CD68P1, CO-029, FGF-5, G250, Ga733EpCAM, HTgp-175, M344, MA-50, MG7-Ag, MOV18, NB/70K, NY-CO-1, RCAS1, SDCCAG1 6, TA-90Mac-2 binding proteincyclophilm C-associated protein, TAAL6, TAG72, TLP, TPS, GPC3, MUC16, LMP1, EBMA-1, BARF-1, CS1, CD319, HER1, B7H6, LICAM, IL6, and MET.

The CAR polypeptide can also comprise a transmembrane domain (such as, for example, an NKG2D transmembrane domain, a CD4 transmembrane domain, a CD8 transmembrane domain, a CD28 transmembrane domain, and/or a CD3 transmembrane domain) and a co-stimulatory domain (such as, for example, a 2B4 domain (e.g., SEQ ID NO: 13), a CD28 co-stimulatory domain (e.g., SEQ ID NO: 7), a 4-1 BB co-stimulatory domain (e.g., SEQ ID NO: 20), or any combination of a 2B4 domain, a CD28 co-stimulatory domain and/or a 4-1 BB co-stimulatory domain). For example, in some embodiments, the CAR polypeptide comprises a IgG4 hinge domain (e.g., SEQ ID NO: 19), a CD4 transmembrane domain, a CD28 co-stimulatory domain (e.g., SEQ ID NO: 7), a CD3zeta polypeptide (e.g., SEQ ID NOs: 8, 15, or 21), and a single-chain variable fragment (scFV) that specifically binds to a receptor on a target cell including, but not limited to, a cancer cell expressing a target antigen (for example, CD33 or CD38). In some embodiments, the CAR polypeptide comprises a IgG4 hinge domain, a NKG2D transmembrane domain, a 2134 domain, a CD3zeta polypeptide, and a single-chain variable fragment (scFV) that specifically binds to a receptor on a target cell including, but not limited to, a cancer cell expressing a target antigen (for example, CD33 or CD38).

In some embodiments, the CD38 scFV described herein comprises a sequence at least about 70% (for example, at least about 75%, 80%, 85%, 90%, 95%, 97%, or 99%) identical to SEQ ID NO: 18, 25, 27, 29, 31 or a fragment thereof.

In some embodiments, the polynucleotide encoding the CD28 co-stimulatory domain described herein comprises a sequence at least about 70% (for example, at least about 75%, 80%, 85%, 90%, 95%, 97%, or 99%) identical to SEQ ID NO: 7 or a fragment thereof.

In some embodiments, the polynucleotide encoding the CD3zeta described herein comprises a sequence at least about 70% (for example, at least about 75%, 80%, 85%, 90%, 95%, 97%, or 99%) identical to SEQ ID NO: 8, SEQ ID NO: 15, SEQ ID NO: 21, or a fragment thereof.

In some embodiments, the polynucleotide encoding the NKG2D transmembrane domain described herein comprises a sequence at least about 70% (for example, at least about 75%, 80%, 85%, 90%, 95%, 97%, or 99%) identical to SEQ ID NO: 11 or a fragment thereof.

In some embodiments, the polynucleotide encoding the 2B4 domain described herein comprises a sequence at least about 70% (for example, at least about 75%, 80%, 85%, 90%, 95%, 97%, or 99%) identical to SEQ ID NO: 13 or a fragment thereof.

In some embodiments, the CD38 scFV described herein comprises a sequence at least about 70% (for example, at least about 75%, 80%, 85%, 90%, 95%, 97%, or 99%) identical to SEQ ID NO: 18, 25, 27, 29, 31 or a fragment thereof.

In some embodiments, the MND promoter described herein comprises a sequence at least about 70% (for example, at least about 75%, 80%, 85%, 90%, 95%, 97%, or 99%) identical to SEQ ID NO: 17 or a fragment thereof.

In some embodiments, the expression vector described herein comprises one or more linker sequences, wherein the linker sequence comprises a sequence at least about 70% (for example, at least about 75%, 80%, 85%, 90%, 95%, 97%, or 99%) identical to SEQ ID NO: 9, SEQ ID NO: 12, or SEQ ID NO: 14, or a fragment thereof.

CD33CAR NK cells and CD38CAR NK cells are shown to be effective to treat cancers (e.g., acute myeloid leukemia). CD38KO/CD33CAR NK cells and CD38KO/CD38CAR NK cells are generated herein and uses thereof are disclosed.

Accordingly, in some embodiments, disclosed herein is an engineered cell (e.g., T cell, NK cell, NKT cell, B cell, or macrophage) comprising a polynucleotide sequence encoding a CD33CAR polypeptide comprising a single-chain variable fragment (scFV) that specifically binds to a CD33 polypeptide, wherein the engineered cell is suppressed in the expression of CD38. In some embodiments, disclosed herein is an engineered cell comprising a polynucleotide sequence a transmembrane domain (e.g., an NKG2D transmembrane domain, a CD4 transmembrane domain, a CD8 transmembrane domain, a CD28 transmembrane domain, or a CD3ξ transmembrane domain), a costimulatory domain (e.g., a 2B4 domain, a CD28 co-stimulatory domain, a 4-1 BB co-stimulatory domain, or any combination of a 2B4 domain, a CD28 co-stimulatory domain and/or a 4-1 BB co-stimulatory, domain), CD3zeta, and a single-chain variable fragment (scFV) that specifically binds to a CD33 polypeptide.

In some embodiments, disclosed herein is an engineered cell (e.g., T cell, NK cell, NKT cell, B cell, or macrophage) comprising a polynucleotide sequence encoding a CD38CAR polypeptide comprising a single-chain variable fragment (scFV) that specifically binds to a CD38 polypeptide, wherein the engineered cell is suppressed in the expression of CD38. In some embodiments, disclosed herein is an engineered cell comprising a polynucleotide sequence a transmembrane domain (e.g., an NKG2D transmembrane domain, a CD4 transmembrane domain, a CD8 transmembrane domain, a CD28 transmembrane domain, or a CD3 transmembrane domain), a costimulatory domain (e.g., a 2B4 domain, a CD28 co-stimulatory domain, a 4-1 BB co-stimulatory domain, or any combination of a 2B4 domain, a CD28 co-stimulatory domain and/or a 4-1 BB co-stimulatory domain), CD3zeta, and a single-chain variable fragment (scFV) that specifically binds to a CD38 polypeptide.

The engineered cells (e.g., T cell, NK cell, NKT cell, or B cell, or macrophage) disclosed herein are generated by methods comprising introducing a double-strand break (DSB) in the genome of the cells, which results in successful gene knock-out and enhanced antitumor activity. After this initial success in gene silencing, the development of a gene insertion method was further pursued. After Cas9 introduces a DSB, two independent and innate DNA repair mechanisms can be employed to repair the break: homologous recombination (HR) or non-homologous end-joining (NHEJ). In the presence of a DNA template encoding a gene of interest, the exogenous gene can be integrated into the Cas9-targeting site using either of these repair mechanisms.

In some embodiments, the engineered cell (e.g., T cell, NK cell, NKT cell, B cell, or macrophage) comprising the chimeric antigen receptor (CAR) is created by a) obtaining a ribonucleoprotein (RNP) complex comprising a class 2 CRISPR/Cas endonuclease (Cas9) complexed with a corresponding CRISPR/Cas guide RNA and an AAV vector comprising a plasmid, nucleic acid, and/or construct comprising a polynucleotide sequence encoding a chimeric antigen receptor (CAR) polypeptide; wherein the polynucleotide sequence is flanked by homology arms; and wherein the homology arms are 800 bp in length or less, and b) introducing the polynucleotide sequence encoding the CAR polypeptide and the RNP complex into a cell; wherein the polynucleotide sequence encoding the CAR polypeptide is introduced into the cell via infection with the AAV into the cell; wherein the RNP complex hybridizes to a target sequence within the genomic DNA of the cell and the cell's DNA repair enzymes insert the polynucleotide sequence encoding the CAR polypeptide into the host genome at the target sequence within the genomic DNA of the cell thereby creating the engineered cell. In some embodiments, the engineered cells are generated by the methods disclosed in International Application Nos. WO2022/093863 and WO2020/198675, which are incorporated herein by reference in their entireties.

Also disclosed herein are methods of making the engineered cell of any preceding aspect comprising the chimeric antigen receptor (CAR) the method comprising: a) obtaining a ribonucleoprotein (RNP) complex comprising a class 2 CRISPR/Cas endonuclease (Cas9) complexed with a corresponding CRISPR/Cas guide RNA and an AAV vector comprising a plasmid, nucleic acid, or construct comprising a polynucleotide sequence encoding a chimeric antigen receptor (CAR) polypeptide; wherein the polynucleotide sequence is flanked by homology arms; and wherein the homology arms are 800 bp in length or less; and b) introducing the polynucleotide sequence encoding the CAR polypeptide and the RNP complex into a cell; wherein the polynucleotide sequence encoding the CAR polypeptide is introduced into the cell via infection with the AAV into the cell; wherein the RNP complex hybridizes to a target sequence within the genomic DNA of the cell and the cell's DNA repair enzymes insert the polynucleotide sequence encoding the CAR polypeptide into the host genome at the target sequence within the genomic DNA of the cell thereby creating the engineered cell.

In some embodiments the homology arms are 10-800 bp in length. In some embodiments, the homology arms are 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, or 800 bp in length.

Further, the homology arms can be symmetrical 30 bp homology arms, symmetrical 300 bp homology arms, symmetrical 500 bp homology arms, symmetrical 600 bp homology arms, symmetrical 800 bp homology arms, or asymmetrical 800 bp homology arms for homologous recombination (HR) or no homology arms at all for non-homologous end joining using homology-independent targeted integration (HITI) plasmids. In some examples, the plasmids with or without homology arms are those disclosed in International Publication Number WO2020/198675, which is incorporated herein by reference in its entirety. It is understood and herein contemplated that homology arms can be symmetrical (same length on each side) or asymmetrical (different lengths on each side) to accommodate differing transgene lengths.

In some embodiments, the left homology arm and right homology arm are the same length. In some embodiments, the homology arms are each 600 bp in length. In some embodiments, the left homology arm and right homology arm are different lengths. In some embodiments, the LHA is 600 bp in length. In some embodiment, the LHA comprises a sequence at least about 70% (for example, at least about 75%, 80%, 85%, 90%, 95%, 97%, or 99%) identical to SEQ ID NO: 2 or 23 or a fragment thereof. In some embodiments, the RHA is 600 bp in length. In some embodiment, the RHA comprises a sequence at least about 70% (for example, at least about 75%, 80%, 85%, 90%, 95%, 97%, or 99%) identical to SEQ ID NO: 3 or 22 or a fragment thereof.

That is, homology arm lengths can have any combination of left homology arm (LHA) length and right homology arm (RHA) length including but not limited to LHA 30 bp and RHA 30 bp, LHA 30 bp and RHA 100 bp, LHA 30 bp and RHA 300 bp, LHA 30 bp and RHA 500 bp, LHA 30 bp and RHA 800 bp, LHA 30 bp and RHA 1000 bp, LHA 100 bp and RHA 30 bp, LHA 100 bp and RHA 100 bp, LHA 100 bp and RHA 300 bp, LHA 100 bp and RHA 500 bp, LHA 100 bp and RHA 800 bp, LHA 100 bp and RHA 1000 bp, LHA 300 bp and RHA 30 bp, LHA 300 bp and RHA 100 bp, LHA 300 bp and RHA 300 bp, LHA 300 bp and RHA 500 bp, LHA 300 bp and RHA 800 bp, LHA 300 bp and RHA 1000 bp, LHA 500 bp and RHA 30 bp, LHA 500 bp and RHA 100 bp, LHA 500 bp and RHA 300 bp, LHA 500 bp and RHA 500 bp, LHA 500 bp and RHA 800 bp, LHA 500 bp and RHA 1000 bp, LHA 800 bp and RHA 30 bp, LHA 800 bp and RHA 100 bp, LHA 800 bp and RHA 300 bp, LHA 800 bp and RHA 500 bp, LHA 800 bp and RHA 800 bp, LHA 800 bp and RHA 1000 bp, LHA 1000 bp and RHA 30 bp, LHA 1000 bp and RHA 100 bp, LHA 1000 bp and RHA 300 bp, LHA 1000 bp and RHA 500 bp, LHA 1000 bp and RHA 800 bp, and LHA 1000 bp and RHA 1000 bp.

There are several ways to provide the DNA template, including viral and non-viral methods. In non-viral approaches, the single-stranded or double-stranded DNA template is typically electroporated along with Cas9/RNP, however, it has a lower efficiency in comparison to viral transduction. For viral gene delivery, adeno-associated viruses (AAV), including AAV6, were used safely in clinical trials and are useful as vectors for sensitive primary immune cells, including T-cells.

Transcripts that are delivered via AAV vectors can be packaged as a linear single-stranded (ss) DNA with a length of approximately 4.7 kb (ssAAV) or as linear self-complementary (sc) DNA (scAAV). The benefit of the scAAV vector is that it contains a mutated inverted terminal repeat (ITR), which is required for replication and helps to bypass rate-limiting steps of second strand generation in comparison to ssDNA vectors.

In some embodiments, a CRISPaint approach is used to create the engineered cell disclosed herein. The CRISPaint approach is a homology-independent method for gene insertion or tagging. In this method, the sequence encoding crRNA and PAM sequence (herein also termed as PAMg) is provided in the DNA template encoding the gene of interest. Upon the introduction of the Cas9 complex, both template and genomic DNA are cut simultaneously. As a result, the CRISPaint template is presented as a linearized double-stranded DNA that can be integrated through non-homology repair machinery. In some embodiments, the CRISPaint templates used herein are those disclosed in International Application No. WO2022/093863, which is incorporated herein by reference in its entirety. Accordingly, in one aspect, disclosed herein are plasmids, nucleic acids, or constructs for delivering donor transgene to a cell and integrating said transgene (e.g., CAR) into the cell in combination with CRISPR/Cas9. Thus, disclosed herein are plasmids, nucleic acids, or constructs for use with CRISPR/Cas9 integration systems of any preceding aspect, wherein the left homology arm and right homology arm are the same length or different lengths.

In some embodiments, the plasmid, nucleic acid, or construct further comprises a murine leukemia virus derived (MND) promoter. In some embodiments, the serotype of the AAV comprises AAV6. In some embodiments, the vector is a single stranded AAV (ssAAV) or a self-complimentary AAV (scAAV).

Accordingly, in some embodiment, the engineered cell (e.g., T cell, NK cell, NKT cell, B cell, or macrophage) disclosed herein comprising the chimeric antigen receptor (CAR) is created by a) obtaining a ribonucleoprotein (RNP) complex comprising a class 2 CRISPR/Cas endonuclease (Cas9) complexed with a corresponding CRISPR/Cas guide RNA and an AAV vector comprising a plasmid, nucleic acid, or construct comprising a polynucleotide sequence encoding a chimeric antigen receptor (CAR); wherein the polynucleotide sequence is adjacent to one protospacer adjacent motifs (PAMs) and one polynucleotide sequence encoding crispr RNA (crRNA) or flanked by one or more PAMs and one or more polynucleotide sequences that encode crRNAs, and b) introducing the polynucleotide sequence encoding the CAR polypeptide and the RNP complex into a cell; wherein the plasmid, nucleic acid, or construct is introduced into the cell via infection with the Adeno-associated virus (AAV) into the cell; wherein in the ribonucleoprotein (RNP) complex hybridizes to a target sequence within the genomic DNA of the cell, and the cell's DNA repair enzymes insert the polynucleotide encoding the CAR into the host genome at the target sequence, thereby creating the engineered cell.

The plasmid, nucleic acid, and/or construct disclosed herein comprises in order one PAM sequence and one polynucleotide sequence that encodes crRNA, the polynucleotide sequence encoding the chimeric antigen receptor CAR polypeptide, one polynucleotide sequence that encodes crRNA, and one PAM sequence. The plasmid, nucleic acid, and/or construct disclosed herein can further comprise in order the polynucleotide sequence encoding the CAR polypeptide, one polynucleotide sequence that encodes crRNA, and one PAM sequence.

Also disclosed herein are plasmids, nucleic acids, or constructs that can be integrated into the genome of the transduced cells via HITI, CRISPaint, or other nonhomologous end joining (NHEJ). As such, they have an advantage of integrating with higher efficiency. In some examples, the plasmids, nucleic acids, or constructs for NHEJ are those disclosed in International Publication Numbers WO2020/198675 and WO2022/093863, which are incorporated herein by reference in their entireties. To aid in the identification of cleavage site to remove the transgene for integration, the plasmids, nucleic acids, or constructs comprise one or more PAMg sequences (i.e., the protospacer adjacent motif (PAM) and the sequence encoding crRNA (i.e., the gRNA)) to target the donor transgene integration. In some examples, for the NHEJ DNA templates (e.g., CRISPaint DNA templates), a single (PAMg) or a double (PAMgPAMg) Cas9-targeting sequences are incorporated around the transgene (e.g., a polynucleotide encoding the CAR, such as CD33 CAR or CD38 CAR, disclosed herein) but within the ITRs. Therefore, Cas9 can simultaneously cut gDNA and the CRISPaint DNA template, enabling integration at the genomic DSB.

Accordingly, in some aspects, disclosed herein is a plasmid, nucleic acid, and/or construct for use with clustered regularly interspaced short palindromic repeat (CRISPR)/CRISPR-associated 9 (Cas9) integration systems wherein the plasmid, nucleic acid, or construct comprises a polynucleotide sequence encoding a chimeric antigen receptor (CAR) polypeptide; wherein the polynucleotide sequence is adjacent to one protospacer adjacent motif (PAM) and one polynucleotide sequence encoding crispr RNA (crRNA) or flanked by two PAMs and two polynucleotide sequences encoding crRNAs. In some aspects, disclosed herein is a plasmid, nucleic acid, and/or construct for use with clustered regularly interspaced short palindromic repeat (CRISPR)/CRISPR-associated 9 (Cas9) integration systems wherein the plasmid, nucleic acid, or construct comprises in order one protospacer adjacent motif (PAM) sequence and one polynucleotide sequence encoding crRNA, a polynucleotide sequence encoding a chimeric antigen receptor (CAR) polypeptide, and one PAM sequence and one polynucleotide sequence encoding crRNA.

In some embodiments, also disclosed herein are methods of making the engineered cell of any preceding aspect comprising the chimeric antigen receptor (CAR) said methods comprising a) obtaining a ribonucleoprotein (RNP) complex comprising a class 2 CRISPR/Cas endonuclease (Cas9) complexed with a corresponding CRISPR/Cas guide RNA and an AAV vector comprising a plasmid, nucleic acid, or construct comprising a polynucleotide sequence encoding a chimeric antigen receptor (CAR); wherein the polynucleotide sequence is adjacent to one protospacer adjacent motifs (PAMs) and one polynucleotide sequence encoding crispr RNA (crRNA) or flanked by one or more PAMs and one or more polynucleotide sequences that encode crRNAs; and b) introducing the polynucleotide sequence encoding the CAR polypeptide and the RNP complex into a cell; wherein the plasmid, nucleic acid, or construct is introduced into the cell via infection with the Adeno-associated virus (AAV) into the cell; wherein in the ribonucleoprotein (RNP) complex hybridizes to a target sequence within the genomic DNA of the cell, and the cell's DNA repair enzymes insert the polynucleotide encoding the CAR into the host genome at the target sequence, thereby creating the engineered cell.

It is understood and herein contemplated that the use of a particular Cas9 can change the PAM sequence which the Cas9 endonuclease (or alternative) uses to screen for targets. As used herein, suitable PAM sequences comprises NGG (SpCas9 PAM) NNGRRT (SaCas9 PAM) NNNNGATT (NmCAs9 PAM), NNNNRYAC (CjCas9 PAM), NNAGAAW (St), TTTV (LbCpf1 PAM and AsCpf1 PAM); TYCV (LbCpf1 PAM variant and AsCpf1 PAM variant); where N can be any nucleotide; V=A, C, or G; Y=C or T; W=A or T; and R=A or G.

### Pharmaceutical Compositions

As described above, the compositions of any preceding aspect can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

In one embodiment, the composition further comprises an anti-CD38 inhibitor. In some embodiment, the anti-CD38 inhibitor is an anti-CD38 antibody (including, but not limited to daratumumab, isatuximab, MOR202, and T AK-079). In some embodiments, the anti-CD38 antibody comprises an Fc domain or does not comprise an Fc domain. In some embodiments, the anti-CD38 antibody comprises daratumumab or isatuximab.

Downregulation of CD38 on MM cells is considered to play an important role in developing resistance to daratumumab. Treatment with agent that increases the expression of CD38 (e.g., ATRA) can overcome this resistance. Accordingly, in some embodiments, the composition further comprises an agent to increase an expression level of CD38. In some examples, the agent comprises all-trans retinoic acid (ATRA).

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Inmunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### Methods of Treatment

The plasmids, vectors, engineered cells, and the pharmaceutical compositions disclosed herein can be used to treat, inhibit, reduce, decrease, ameliorate, and/or prevent any disease where uncontrolled cellular proliferation occurs such as cancers.

Thus, disclosed herein are methods of treating, decreasing, reducing, inhibiting, ameliorating, and/or preventing a cancer and/or metastasis (such as, for example, acute lymphocytic leukemia (ALL), acute myeloid leukemia (AML), chronic myeloid leukemia (CML), hairy cell leukemia (HCL), and/or myelodysplastic syndromes (MDS)) in a subject comprising administering to a subject with a cancer any modified cell (for example, engineered NK cells, NK T cells, T cells, or B cells) disclosed herein. For example, disclosed herein are methods of treating, decreasing, reducing, inhibiting, ameliorating, and/or preventing a cancer and/or metastasis (such as, for example, acute lymphocytic leukemia (ALL), acute myeloid leukemia (AML), chronic myeloid leukemia (CML), hairy cell leukemia (HCL), and/or myelodysplastic syndromes (MDS)) in a subject comprising administering to the subject a therapeutically effective amount of an engineered cell (for example, engineered NK cell, NK T cell, T cell, or B cell), wherein the engineered cell (for example, engineered NK cell, NK T cell, T cell, or B cell) comprises a plasmid, nucleic acid, or construct for use with clustered regularly interspaced short palindromic repeat (CRISPR)/CRISPR-associated 9 (Cas9) integration systems wherein the plasmid, nucleic acid, or construct comprises in order a left homology arm, a polynucleotide sequence encoding a chimeric antigen receptor (CAR) polypeptide (such as, for example, a CD33 targeting CAR or CD38 targeting CAR including, but not limited to a CAR comprising the amino acid sequence as set forth in SEQ ID NO: 18, SEQ ID NO: 25, SEQ ID NO: 27, SEQ ID NO: 29, or SEQ ID NO: 31), and a right homology arm; wherein the left and right homology arms are each 1000 bp in length or less (for example, 600 bp).

In some embodiments, the method further comprises administering to the subject a therapeutically effective amount of an anti-CD38 inhibitor. In some embodiments, the anti-CD38 is an anti-CD38 antibody (including, but not limited to daratumumab, isatuximab, MOR202, and TAK-079. In some embodiments, the anti-CD38 antibody comprise an Fc domain. In some embodiments, the anti-CD38 antibody does not comprise an Fc domain.

In some embodiments, the method further comprises administering to the subject a therapeutically effective amount of an agent to increase an expression of CD38 (e.g., all-trans retinoic acid (ATRA)).

The plasmids, vectors, engineered cells, and the pharmaceutical compositions disclosed herein can also be used to treat, inhibit, reduce, decrease, ameliorate, and/or prevent minimal residual disease (MRD). “Minimal residual disease”, “measurable residual disease”, or MRD refers to a very small number of cancer cells that remain in the body during or after treatment. MRD can be found by highly sensitive laboratory methods (e.g., next generation genetic sequencing that analyzes samples genetic mutations or flow cytometry) that are able to find one cancer cell among one million normal cells. MRD testing is used mostly for blood cancers such as lymphoma and leukemia.

In some aspects, disclosed herein is a method of treating, inhibiting, reducing, decreasing, ameliorating, and/or preventing minimal residual disease (MRD) in a subject in need thereof, comprising administering to the subject a therapeutically effective amount of the engineered cell disclosed herein. In some embodiments, the engineered cell comprises a nucleic acid sequence encoding chimeric antigen receptor (CAR) comprising a single-chain variable fragment (scFV) that specifically binds to a target molecule (e.g., CD33CAR or CD38 CAR including, but not limited to a CAR comprising the amino acid sequence as set forth in SEQ ID NO: 18, SEQ ID NO: 25, SEQ ID NO: 27, SEQ ID NO: 29, or SEQ ID NO: 31), wherein the engineered cell is suppressed in the expression of CD38. In some embodiments, the method further comprises administering to the subject a therapeutically effective amount of an anti-CD38 inhibitor. In some embodiments, the anti-CD38 inhibitor is an anti-CD38 antibody (including, but not limited to daratumumab, isatuximab, MOR202, and TAK-079). In some embodiments, the anti-CD38 antibody comprise an Fc domain. In some embodiments, the anti-CD38 antibody does not comprise an Fc domain.

The subject having MRD can be previously treated with one or more anti-cancer therapeutics. Exemplary cancers include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As noted above, the plasmids, nucleic acids, constructs, vectors, and the engineered cell (for example, engineered NK cell, NK T cell, T cell, or B cell) disclosed herein can be used to treat, inhibit, reduce, decrease, ameliorate, and/or prevent cancer. A representative but non-limiting list of cancers that the disclosed compositions can be used to treat is the following: lymphoma, B cell lymphoma, T cell lymphoma, mycosis fungoides, Hodgkin's Disease, acute lymphocytic leukemia (ALL), hairy cell leukemia (HCL), myelodysplastic syndromes (MDS), myeloid leukemia (including, but not limited to acute myeloid leukemia (AML) and chronic myeloid leukemia (CML)), bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

As noted throughout the present disclosure, the disclosed the engineered cells (for example, engineered NK cells, NK T cells, T cells, or B cells) are ideally suited for use in immunotherapy such as the adoptive transfer of the engineered (i.e., engineered NK cells, NK T cells, T cells, or B cells to a subject in need thereof). Thus, in one aspect, disclosed herein are methods of adoptively transferring an engineered cell to a subject in need thereof said method comprising a) obtaining a cell to be engineered; b) obtaining a ribonucleoprotein (RNP) complex comprising a class 2 CRISPR/Cas endonuclease (Cas9) complexed with a corresponding CRISPR/Cas guide RNA and an AAV vector comprising a plasmid, nucleic acid, or construct comprising a transgene (such as, for example, a chimeric antigen receptor for a tumor antigen); wherein the transgene is flanked by homology arms; and wherein the homology arms are less than 1000 bp; and c) introducing the transgene and the RNP complex into the cell; wherein the transgene is introduced into the cell via infection with the Adeno-associated virus (AAV) into the cell; wherein the RNP complex hybridizes to a target sequence within the genomic DNA of the cell and the cell's DNA repair enzymes insert the transgene into the host genome (for example, by homologous repair) at the target sequence within the genomic DNA of the target cell thereby creating an engineered cell; and d) transferring the engineered cell into the subject. In one aspect the transgene can be comprised on the same plasmid, nucleic acid, or construct as the Cas9 endonuclease or encoded on a second plasmid, nucleic acid, or construct in the same or different AAV vector. In one aspect, the target cell can be transduced with the RNP complex via electroporation before or concurrently with the infection of the cell with the transgene comprising AAV.

In one aspect, the engineered cell (e.g., engineered NK cells, NK T cells, T cells, or B cells) used in the disclosed immunotherapy methods can be primary cells from a donor source (such as, for example, an allogeneic donor source for an adoptive transfer therapy or an autologous donor source (i.e., the ultimate recipient of the modified cells), a cell line (including, but not limited to NK cell lines NK RPMI8866; HFWT, K562, and EBV-LCL), or from a source of expanded cells derived a primary cell source or cell line. Because primary cells can be used, it is understood and herein contemplated that the disclosed modifications of the cell can occur ex vivo or in vitro.

Also disclosed herein is a plasmid, nucleic acid, and/or construct comprising in order a left homology arm, a polynucleotide sequence encoding a chimeric antigen receptor (CAR) polypeptide, and a right homology arm; wherein the left and right homology arms are each 1000 bp in length or less.

In one aspect, disclosed herein are a plasmid, nucleic acid, construct, an AAV vector, or a modified cell as disclosed herein for use as a medicament. Also disclosed herein are a use of a plasmid, nucleic acid, and/or construct, an AAV vector or a modified cell as disclosed herein for the manufacture of a medicament.

Also disclosed herein are a plasmid, nucleic acid, construct, an AAV vector or a modified cell as disclosed herein for use in the treatment of cancer. Also disclosed herein are a use of a plasmid, nucleic acid, construct, an AAV vector, or a modified cell as disclosed herein for the manufacture of a medicament for the treatment of cancer.

Also disclosed herein are a CAR cell (e.g., engineered NK cell, NK T cell, or T cell), created by using a method of creating a chimeric antigen receptor (CAR) natural killer (NK) cell, NK T cell, or T cell as disclosed herein, for use in the treatment of cancer. Also disclosed herein are a use of a CAR cell (e.g., engineered NK cell, NK T cell, or T cell), created by using a method of creating a chimeric antigen receptor (CAR) engineered NK cell, NK T cell, or T cell as disclosed herein, for the manufacture of a medicament for the treatment of cancer.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Combination CD38^{KO}/CAR^{KI} NK Cell Immunotherapy for Dual Targeting with CD38 Monoclonal Antibodies

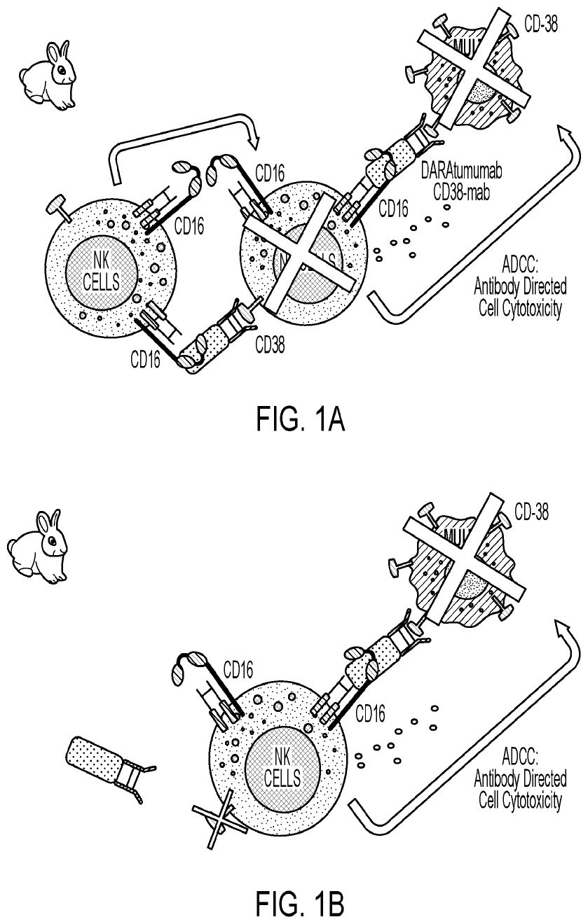

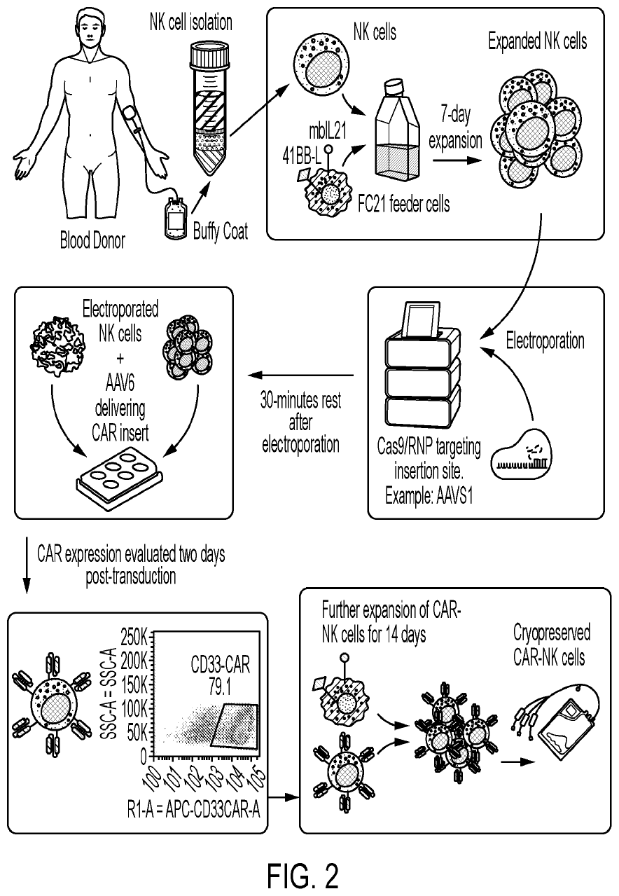

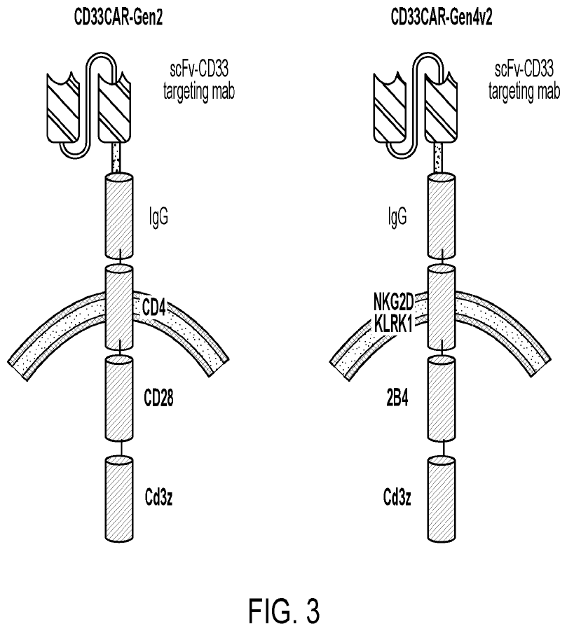

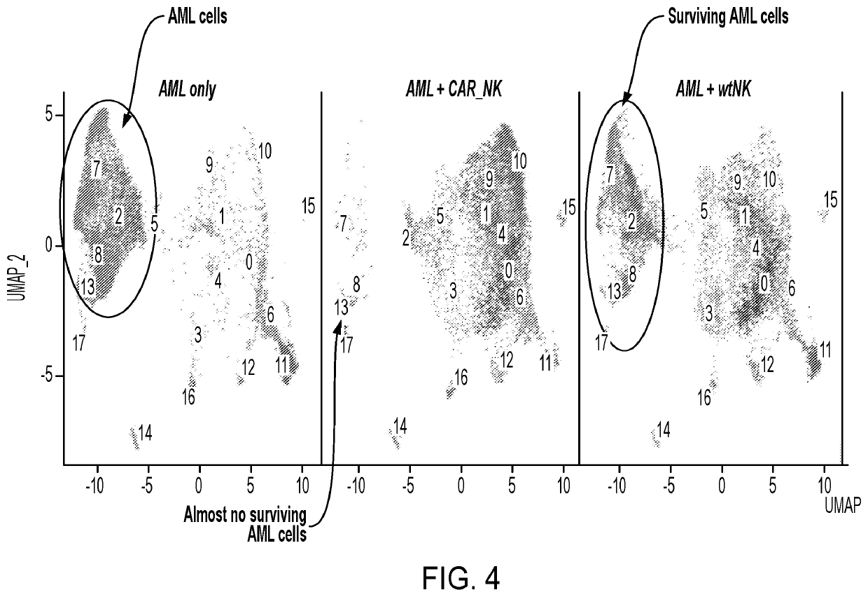

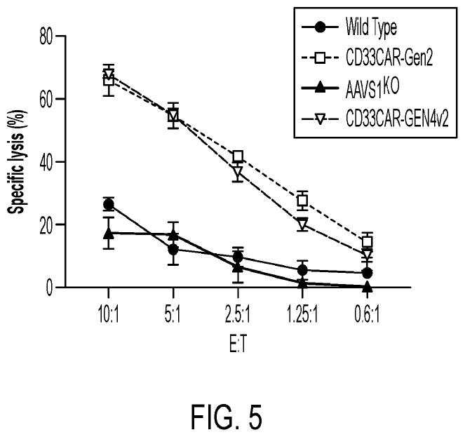

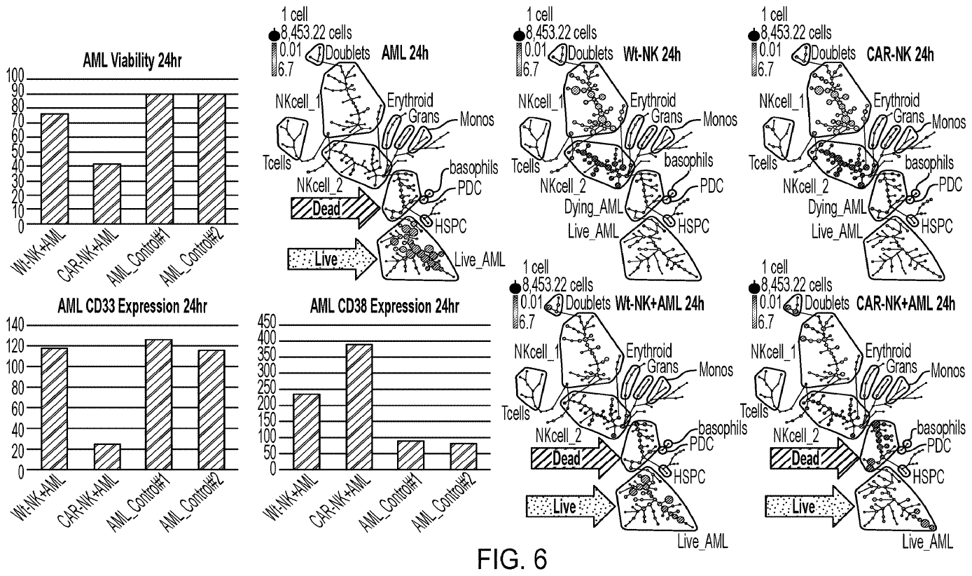

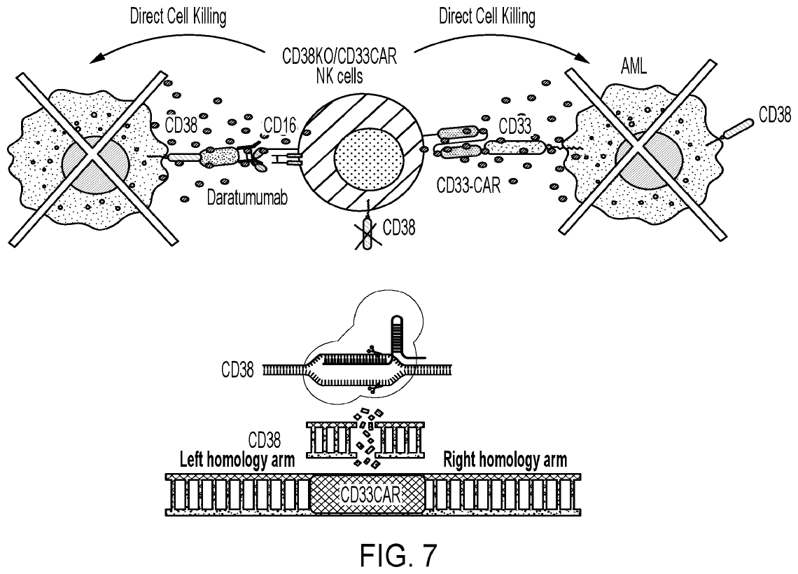

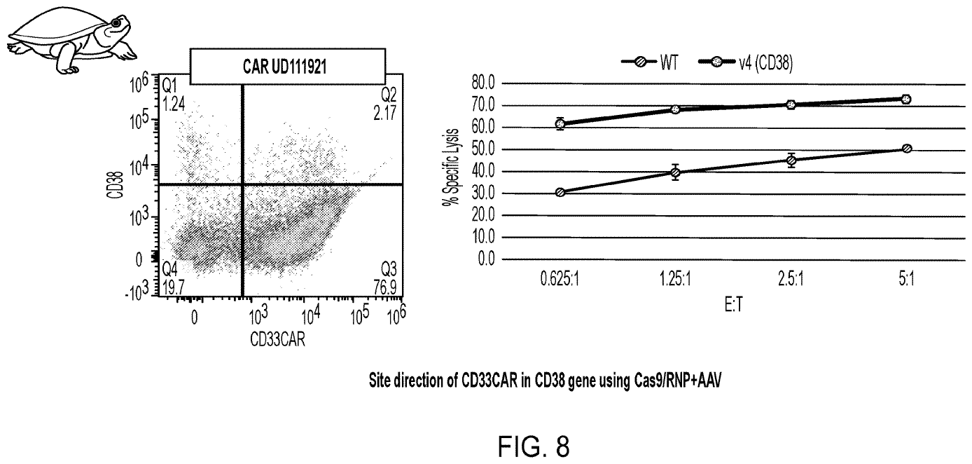

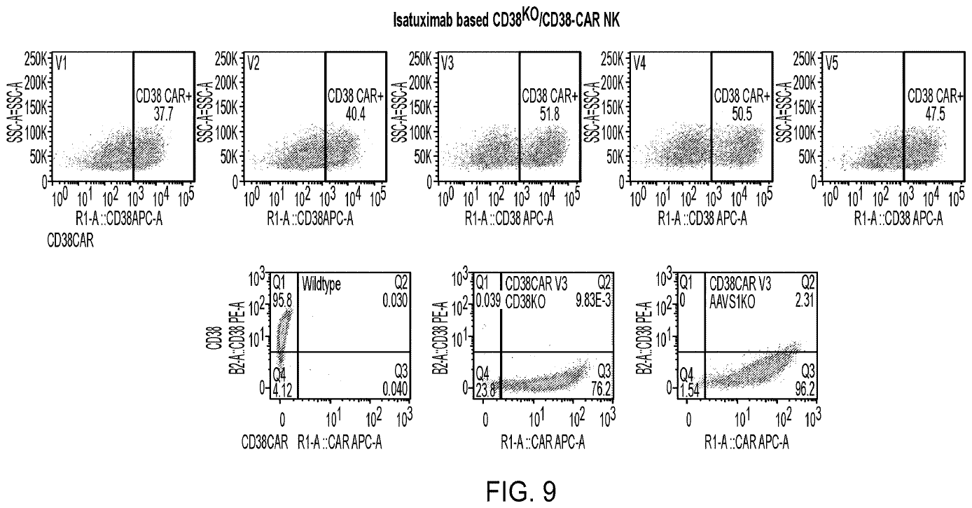

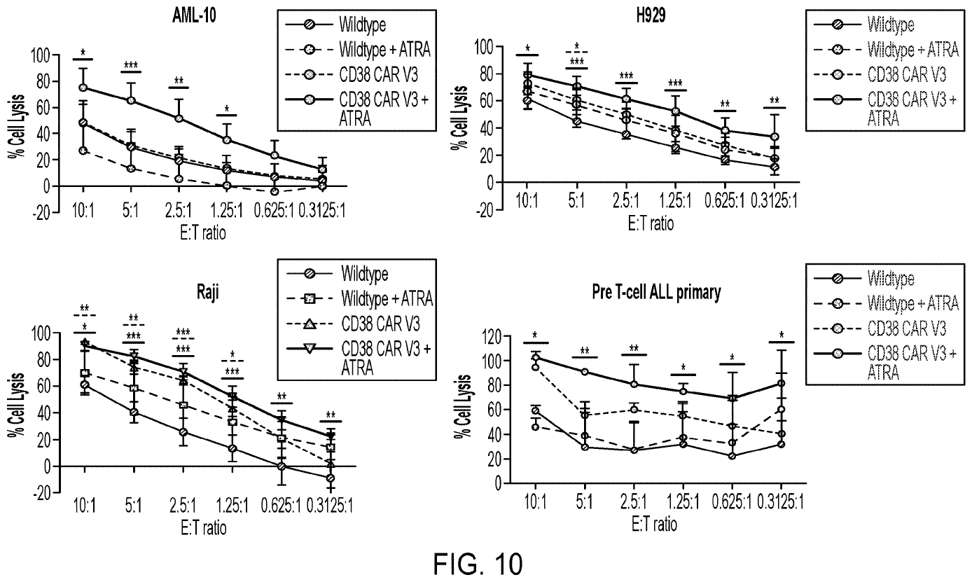

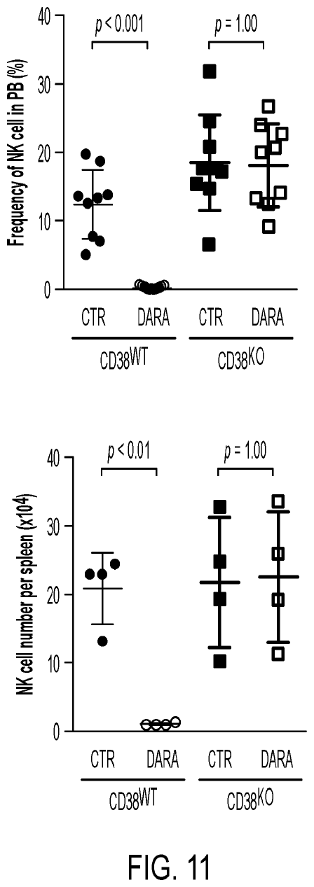

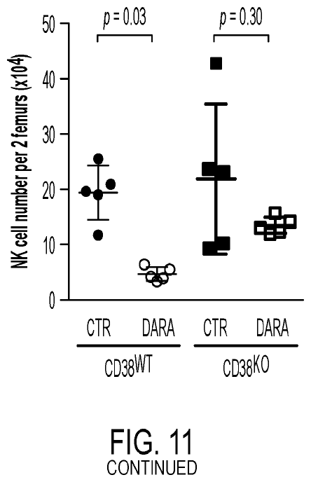

Acute Myeloid Leukemia (AML) is a blood malignancy with high mortality rate. The AML cells highly express CD33 which can be targeted by CD33-CAR expressing immune cells such as NK cells. CD33CAR NK is generated by a combination of Cas9/RNP and AAV cells to effectively target the CD33 expressing AML cells (FIG. 1). However, it has been revealed that AML cells that survive being killed by CD33CAR NK cells have high expression CD38 (FIG. 2). Therefore, using CD38 monoclonal antibodies such as daratumumab can be used to eliminate these cells (Haematologica. 2019 March; 104(3): e100-e103, ClinicalTrials.gov Identifier: NCT03067571). But due to the fact that NK cells themselves have high expression of CD38, CD38 monoclonal antibodies also induce fratricide of NK cells and eliminate them from peripheral blood. It has also been shown that knocking out CD38 on NK cells can overcome the fratricide and boost their antitumor activity (FIG. 3).

Here, NK cells were generated by simultaneous knock-out of CD38 and knock-in of CD33CAR (CD38KO/CD33CARKI− NK). These cells can be used in combination with CD38 monoclonal antibodies with no risk of fratricide in order to eliminate CD38^{positive}CD33^{negative} AML cells which survive CD33CAR-NK cells mediated killing.

### Example 2: CD38-CAR Human NK Cells in Combination with ATRA Enhance Cytotoxicity Against CD38-Expressing Hematologic Malignancies

Cellular therapy has shown tremendous efficacy against hematologic malignancies. Despite the strong anti-tumor efficacy of chimeric antigen receptor (CAR)-T cell therapies, these therapies are limited by associated toxicities and their inability to be accessible, off-the-shelf treatments. Natural killer (NK) cells are immune cells with an innate killing potential toward malignant and viral-infected cells that downregulate major histocompatibility complex (MHC) class I. Thus, natural killer (NK) cell-based immunotherapies offer a unique, off-the-shelf therapeutic option with innate cytotoxicity toward cancerous cells that also have the ability to be further enhanced by genetic engineering to express CARs. Malignant cells can evade NK cell killing by suppressing the expression of activating ligands, preserving MHC expression to inhibit activation, and inhibiting immune cell function through suppressive cytokines. By genetically engineering them to express a CAR, NK cells can directly target tumor-specific antigens and kill independently of MHC recognition or expression levels. Thus, CAR NK cells are a promising therapeutic for hematologic malignancies because they offer target-specific cellular cytotoxicity and retain their innate killing potential.

CD38 is a transmembrane glycoprotein highly expressed on the surface of plasma cells and other lymphoid and myeloid cell populations. CD38 is expressed in high levels on various normal hematologic tissues and on hematologic malignancies such as multiple myeloma (MM), burkitt lymphoma (BL), acute myeloid leukemia (AML), and T-cell leukemias. While these malignancies are diverse in their origin, their high CD38 expression makes them all strong targets for anti-CD38 CAR NK cell therapy. Similarly, CD38 is well-studied to play a role in tumor progression and the tumor microenvironment (TME) by metabolizing NAD+ in hypoxic conditions and assisting in angiogenesis. CD38 targeting cellular therapies are relevant because of their broad application toward a diverse range of CD38 expressing hematologic malignancies.

Currently, CD38 has been targeted in patients with MM using the FDA-approved anti-CD38 monoclonal antibodies, isatuximab and daratumumab. Isatuximab-based combination therapies display efficacy and a tolerable safety level in patients. NK cells play an important role in the recognition and killing of malignant cells in the presence of anti-CD38 monoclonal antibodies through antibody-dependent cellular cytotoxicity (ADCC). However, because NK cells also highly express CD38, anti-CD38 mAbs cause NK NK cells to target and kill one another (a.k.a., fratricide), limiting their efficacy. The limited efficacy of CD38 mAb therapies indicates that novel approaches are needed to reduce fratricide and improve NK cell cytotoxicity toward CD38 malignancies.

### Materials and Methods

### Cell Lines and Primary Cells

CSTX002 feeder cells, derived from K562 and transduced to express membrane-bound IL-21 and 4-1BBL (referred to hereafter as FC21) were cultured in RMPI 1640 media containing 10% fetal bovine serum (FBS) at 37° C., 5% CO2. FC21 were treated with 100 Gy x-ray irradiation (Faxitron, Tucson AZ) prior to coculture with NK cells. Human AML cell line MV4-11, MM cell lines MM1S and H929, and BL cell lines Daudi and Raji were purchased from American type culture collection (ATCC). Primary human AML cells (AML10) were obtained from a pediatric patient with relapsed M5 AML and passaged in NOD-SCID mice. Primary human AML, Pre-T ALL, T-PLL, MM, and HSTCL were obtained from the Ohio State University Comprehensive Cancer Center Leukemia Tissue Bank. Tumor cell lines were cultured in RPMI 1640 media plus Glutamax, 10% FBS and routinely tested for mycoplasma contamination. Anonymized human buffy coats were obtained from the American Red Cross of Ohio (Columbus, OH, USA) as healthy donor lymphocyte source material, designated not human subjects research by the institutional review board.

### NK Cell Purification and In Vitro Expansion

Human primary NK cells were isolated from buffy coats by negative depletion using RosetteSep™ Human NK Cell Enrichment Cocktail (Stem Cell Technologies) and expanded. Briefly, isolated NK cells were stimulated and expanded using FC21 cells. On day 7, NK cells were electroporated and transduced as described below and then rested in NK cell media for 2 days. The genetically modified NK cells were then further expanded by restimulation with FC21 cells (1:1) every 7 days under the culture conditions described above.

### T Cell Purification and In Vitro Expansion

Human primary T cells were isolated from donor buffy coats using RosetteSep™ Human T Cell Enrichment Cocktail (Stem Cell Technologies, Vancouver, BC, Canada) as described for NK cells. Isolated T cells were stimulated on day 0 with Human T-Activator CD3/CD28 (Thermo Fisher Scientific). Stimulated T cells were cultured in RPMI 1640 media plus Glutamax, 10% FBS at a concentration of 1.0e6 cells/mL and supplemented with 50 IU/mL human recombinant IL-7 and IL-15 (Biolegend, San Diego, CA) every 2-3 days.

Two days after cytokine activation the T cells were electroporated and transduced as described below. After transduction the cells were expanded in RPMI 1640 media plus Glutamax, 10% FBS at 1.0e6 cells/mL with IL-7 and IL-15 supplemented at 50 IU/mL every 2-3 days for up to 10 days.

### Generation of Genome-Edited NK and T Cells

CD38^{KO}/CD38-CAR T cells or NK cells were generated by electroporation of HiFi Cas9 Nuclease V3 (Integrated DNA Technologies, Coralville, IA) and gRNA targeting exon 1 of the CD38 gene (5-CTGAACTCGCAGTTGGCCAT) (SEQ ID NO: 1). Knock-out cells were then returned to culture media and transduced within 30 minutes with 75,000 vg/cell of Adeno-Associated Virus Type 6 (AAV6, Andelyn Biosciences) vector containing the CAR expression cassette with CD38 homology arms. Similarly, AAVS1^{KO}/CD38-CAR NK cells were generated using gRNA targeting the AAVS1 safe harbor gene (5-GGGGCCACTAGGGACAGGAT) (SEQ ID NO: 5) and AAV6 vectors with homology arms for the AAVS1 locus.

### Flow Cytometry

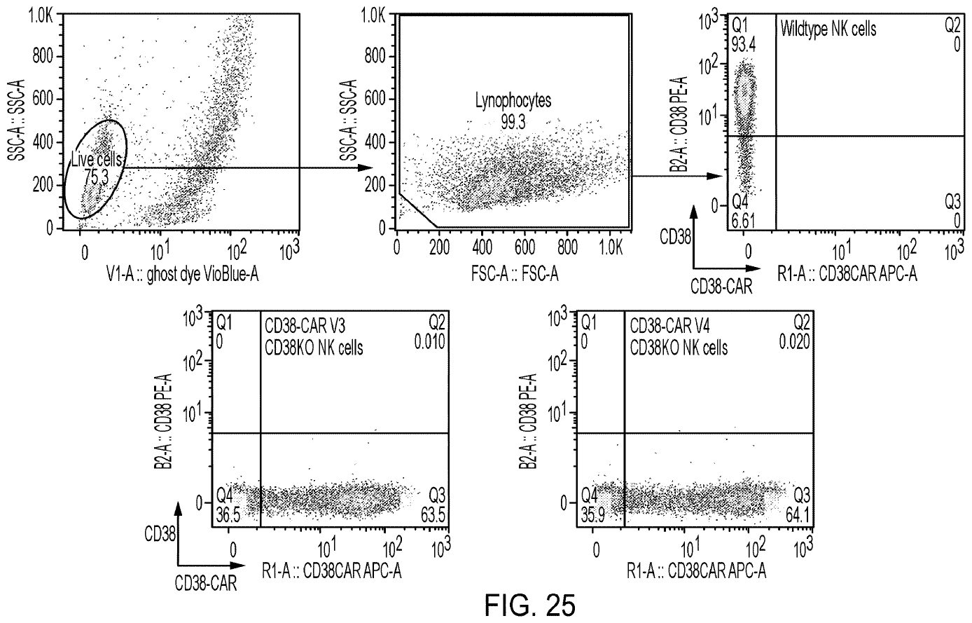

Cells were stained in PBS buffer containing 2% FBS using anti-CD38/PE (Miltenyi Biotec, clone REA572), CD38 Fc Chimera Protein (R&D Systems, rhCD38) followed by secondary staining with Alexa Fluor 647 AffiniPure Goat Anti-Human IgG (Jackson ImmunoResearch), and Tonbo Ghost Dye 450 (Tonbo Biosciences, San Diego, CA, USA). See representative gating strategy (FIG. 25).

### Cytotoxicity Assay

4-hour cytotoxicity assays were performed using calcein AM (Fisher Scientific, Hampton, NH). Briefly, target tumor cells were stained with 2 μg/mL calcein AM and co-cultured with effector cells at the indicated Effector:Target ratios. After 4 hours, culture supernatant was assessed by fluorimetry and percent specific lysis calculated using maximum and minimum controls. Cytotoxicity is plotted as percent specific lysis.

### Metabolic Assay

The oxygen consumption rate (OCR) and extracellular acidification rate (ECAR) of WT, CD38^{K}o/CD38-CAR, and AAVS1^{KO}/CD38-CAR NK cells were measured using the Agilent Extracellular Flux Assay Kit (Aligent Technologies, Santa Clara, CA) on the Seahorse XFe24 analyzer (Aligent Technologies, Santa Clara, CA) according to manufacturer's instructions.

### Bio-Plex Cytokine Assay

Cytokine secretion was assessed in wildtype and CD38^{KO}/CD38-CAR NK cells after co-culture with three separate tumor targets (MMIS, MV4-11, and Raji). Cells were co-cultured for 6 hours at a 5:1 Effector:Target ratio before the supernatant was collected. The assay was performed according to manufacturer instructions (Bio-Plex Pro Human Cytokine 8-plex Assay, Bio-Rad) on the Bioplex 200 system.

### Mass Cytometry Lysis Assay

A primary AML patient cell sample (OSU leukemia tissue bank) was cultured in a 6-well plate (pre-coated with rat tail collagen [Corning]) in serum free media (SFEM) supplemented with a cytokine cocktail (IL-6, IL-3, FLT3, GMCSF, GCSF, and TPO at 6.66 ng/mL, EPO at 3.33 ng/mL, SCF at 20 ng/mL; Stem Cell Technologies). The primary AML samples were cultured for 24 hours with or without 10 nM ATRA. After 24 hours, wild-type or CD38^{KO}/CD38 CAR-NK cells were added at a ratio of 1:1 and co-cultured for 24 hours. Control wells were cultured in parallel. Cell were stained and events were acquired on the Helios CyTOF system (Fluidigm, Toronto, Canada). Cells were identified using markers and analyzed using SPADE.

CD38 qPCR

qPCR analysis on RNA isolated from wildtype and AAVS1^{KO}/CD38-CAR NK cells was performed.

### Statistical Analysis

Statistical analyses were performed using Prism 9.3.0 (GraphPad Software, Inc.) as described in the figure legends. Student t-test was used to compare 2 independent groups. Three or more groups were compared with 2-way ANOVA analysis. p Values less than 0.05 were considered significant.

### Results

### Generation of Highly Efficient CD38^{KO}/CD38CAR NK Cells Using Cas9/RNP and AAV

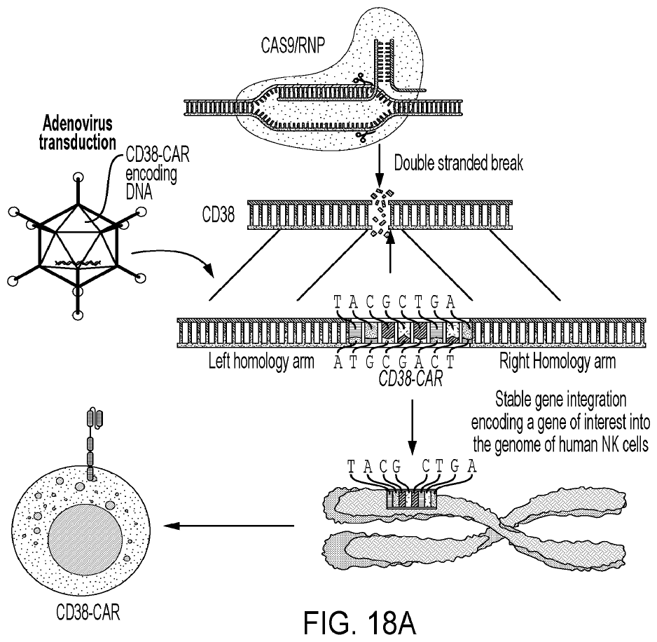

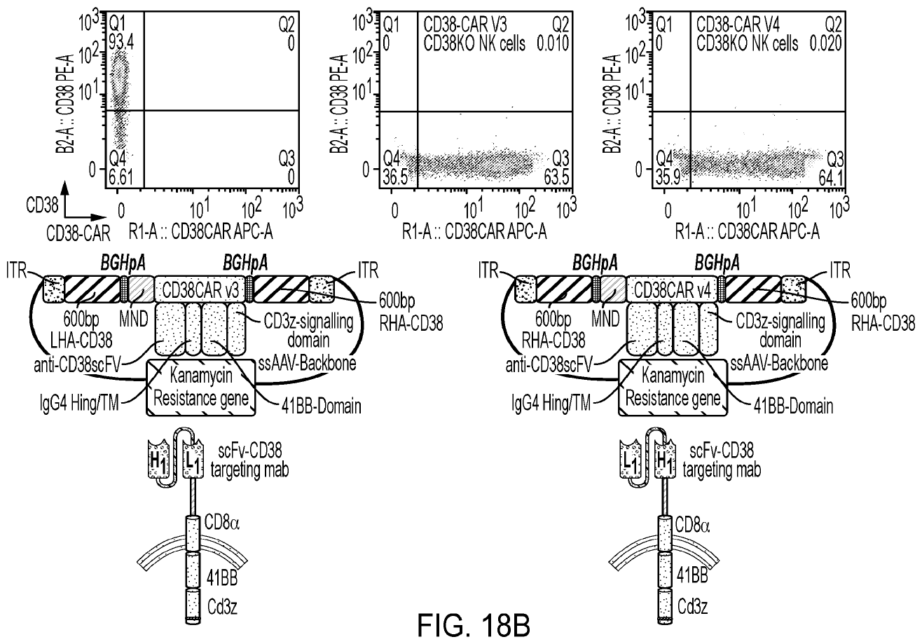

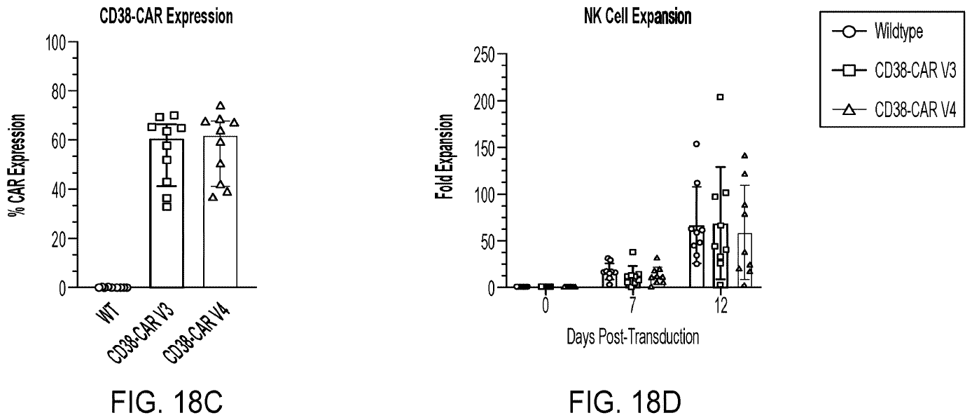

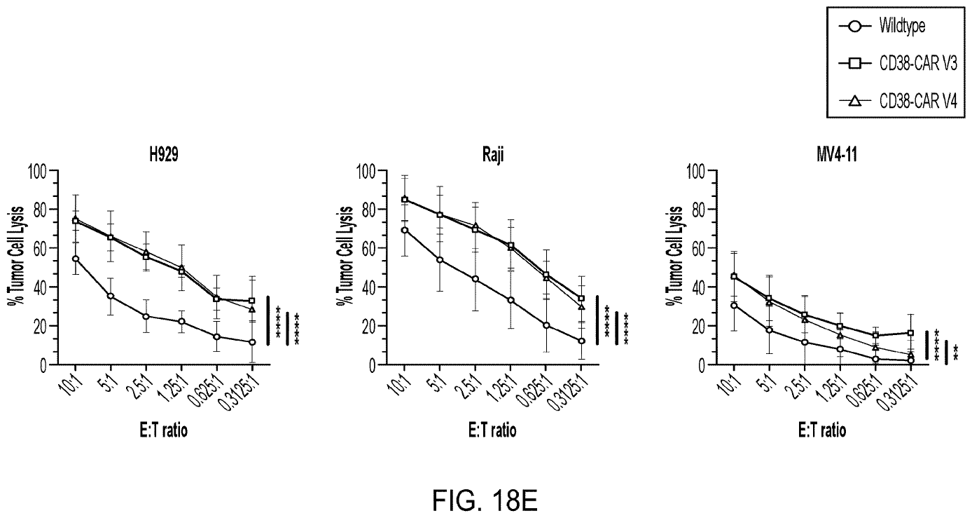

We generated two single-chain variable fragments (scFv) based on publicly-available variable heavy (V_{H}) and light (V_{L}) chains of isatuximab to generate a second-generation CAR with CD3ζ and 41BB co-stimulatory domains and a CD80α transmembrane (TM)/hinge (FIG. 18B). CAR construct V3 expressed the scFv in V_{H}-V_{L} orientation and construct V4 expressed the scFv in V_{L}-V_{H} orientation. After the initial seven-day expansion, activated NK cells were electroporated with Cas9/RNP targeting CD38 and were transduced with AAV vectors providing the CAR constructs with homology arms for the targeting site at the CD38 locus (FIG. 18A). Complete CD38 knockouts and CAR expression were by flow cytometry seven days post-transduction in the CAR NK cells, indicating a successful generation of the CD38^{KO}/CD38-CAR NK cells (FIG. 18B). The median adenovirus transduction efficiency for V3 was 60.7% and for V4 was 61.9% (FIG. 18C). No significant difference between transduction efficiency was observed between V3 and V4 CAR NK cells, so the remaining experiments were all performed with V3.

### CD38^{KO}/CD38-CAR NK Cells Avoid Fratricide and Show Enhanced Cytotoxicity Toward Cancer Cell Lines

NK cells mediate the anti-leukemic effect of anti-CD38 mAbs via antibody-dependent cell-mediated cytotoxicity (ADCC), but their efficacy is also limited by ADCC against each other, known as fratricide. We hypothesized that we could overcome fratricide in NK cells expressing a CD38 CAR by developing a simultaneous CD38 KO and CD38-CAR KI. There was no significant difference between the post-transduction expansion (FIG. 18D) of the CAR-NK cells compared to wildtype NK cells, indicating that the CD38^{KO}/CD38-CAR NK cells avoided fratricide during expansion.

We then performed killing assays to assess the activity of V3 and V4 CARs against CD38-expressing malignancies, and observed a significant increase in cytotoxicity for the CAR-NK cells against all tumor targets (MM (H929), BL (Raji), and AML (MV4-11)) for five NK cell donors. (FIG. 18E). No significant difference was seen between NK cells expressing the V3 and V4 CAR constructs, indicating that the heavy and light chain order did not impact CD38 recognition.

CD38-CAR NK Cells have Enhanced Killing Toward Patient Tumor Samples

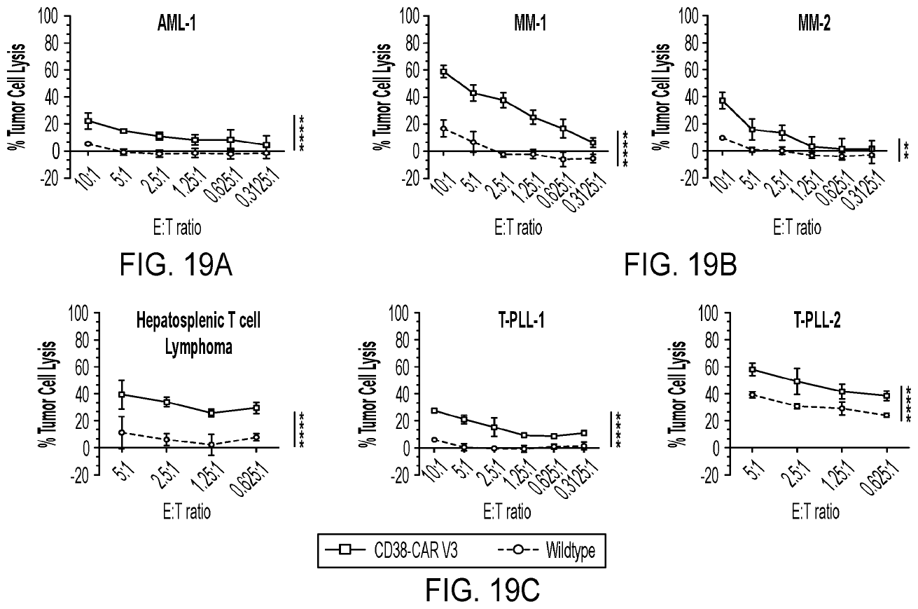

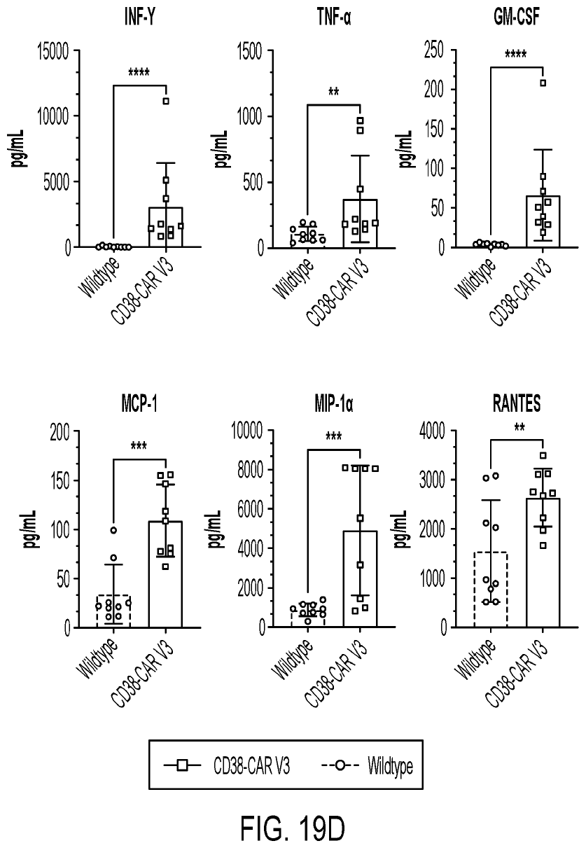

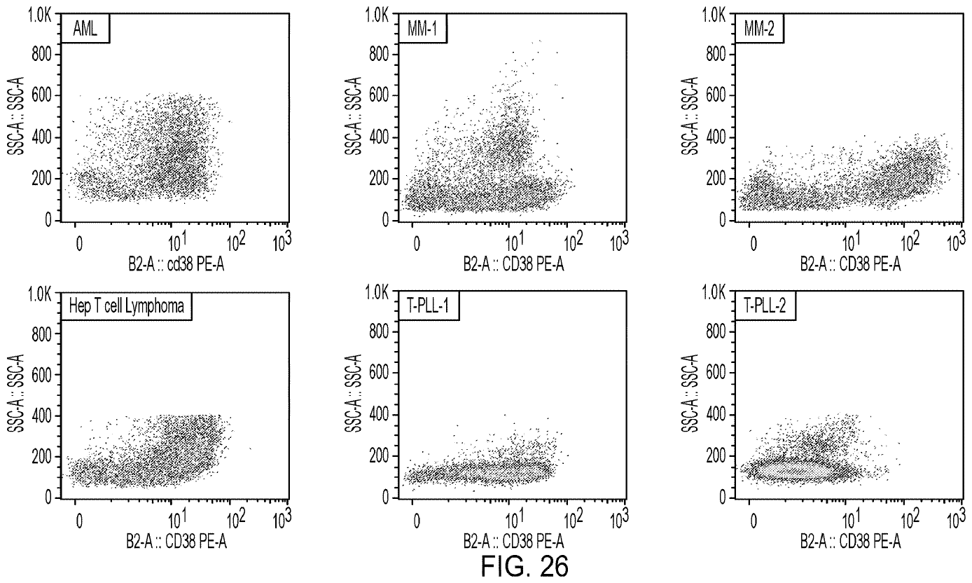

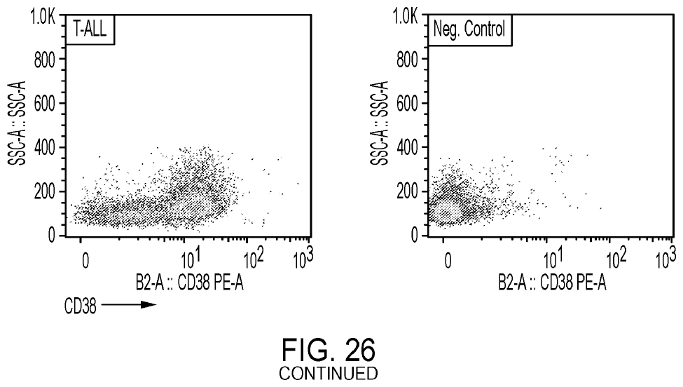

To validate the clinical potential of CD38-CAR NK cells, we tested their cytotoxicity against CD38-expressing (FIG. 26) primary patient tumor samples. One to three NK cell donors were tested against each tumor sample based on the availability of viable primary tumor cells after thawing. CD38-CAR NK cells showed higher killing than WT NK cells against AML (AML-1 (n=3 NK cell donors), FIG. 19A), multiple myeloma (MM-1 and MM-2 (n=1 NK cell donor), FIG. 19B), and lymphoma (T-PLL-1 and T-PLL-2 (n=1 NK cell donor), and HSTCL (n=1 NK cell donor), FIG. 19C).

CD38-CAR NK Cells have Higher Production of Activating Cytokines

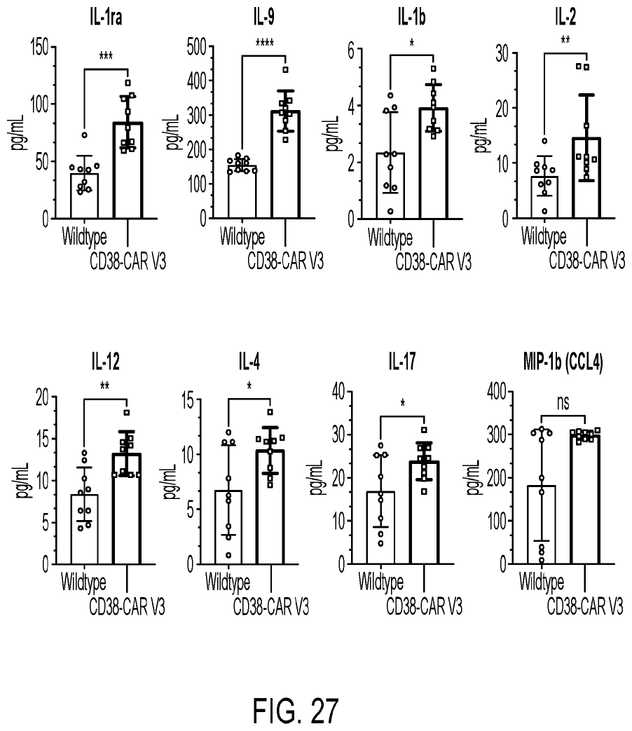

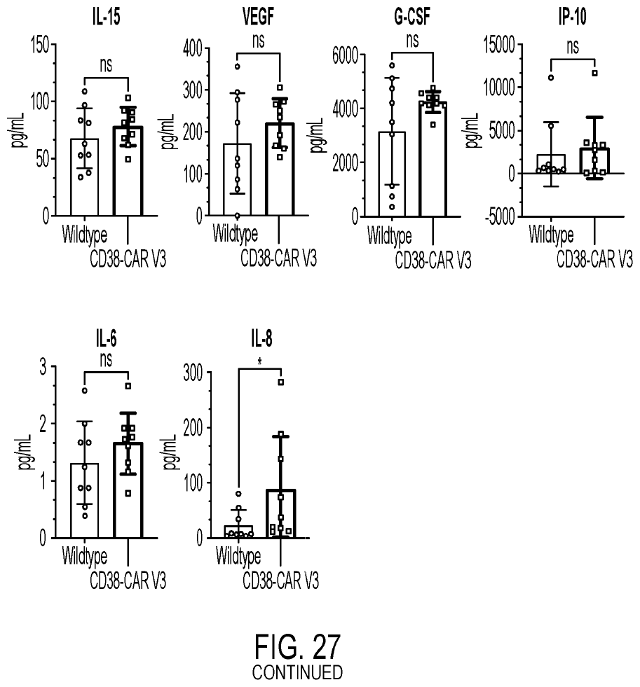

To further evaluate the enhanced activation in the CAR NK cells, we measured the supernatant for cytokines after CAR-NK cells from three different donors were co-cultured with three tumor targets (MMIS, Raji, and MV4-11). The CAR NK cells showed significantly higher production of IFN-γ, TNF-α, GM-CSF, MCP-1, MIP-1α, and RANTES (FIG. 19D), important mediators of NK cell effector function and antitumor activity. They also showed a notable increase in the NK cell activating cytokine IL1RA and IL-9 (FIG. 27). Other cytokines analyzed showed no significant difference (FIG. 27).

### ATRA Increases CD38 Expression and Improves CAR Killing in Certain Cancer Cell Lines

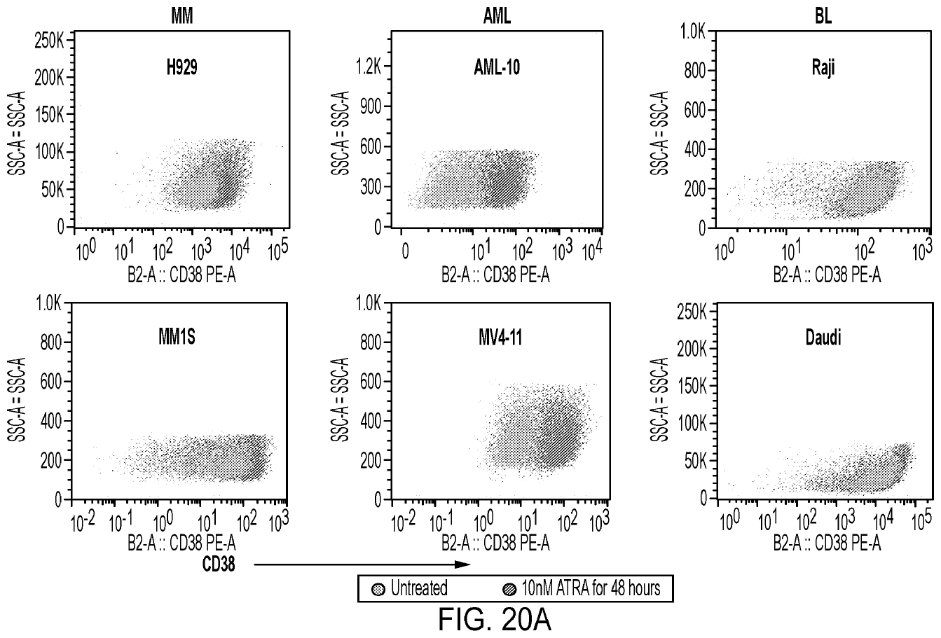

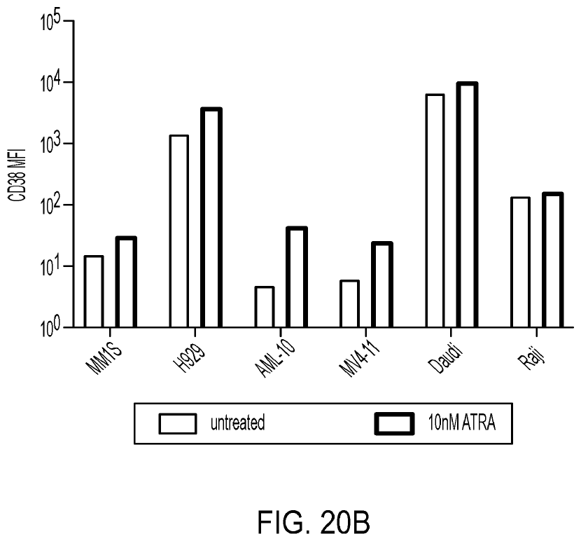

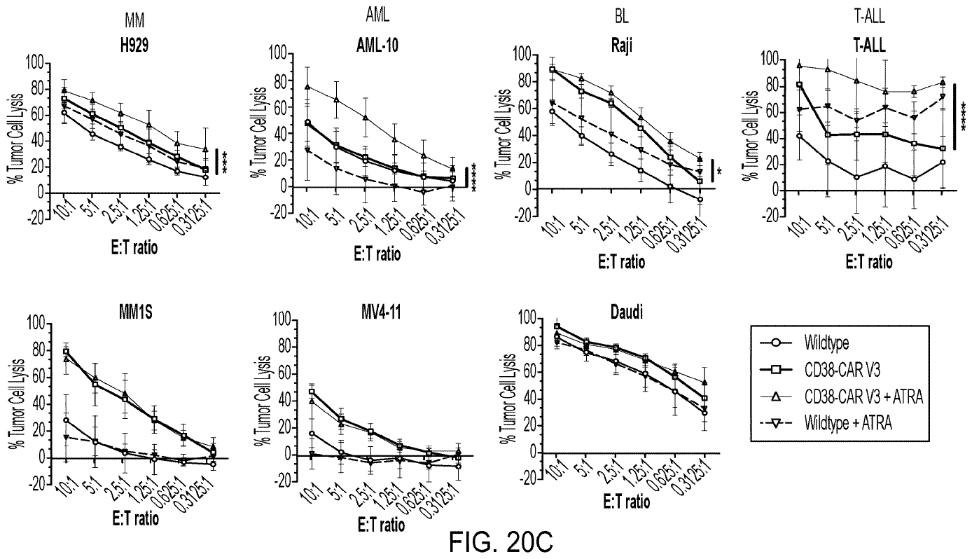

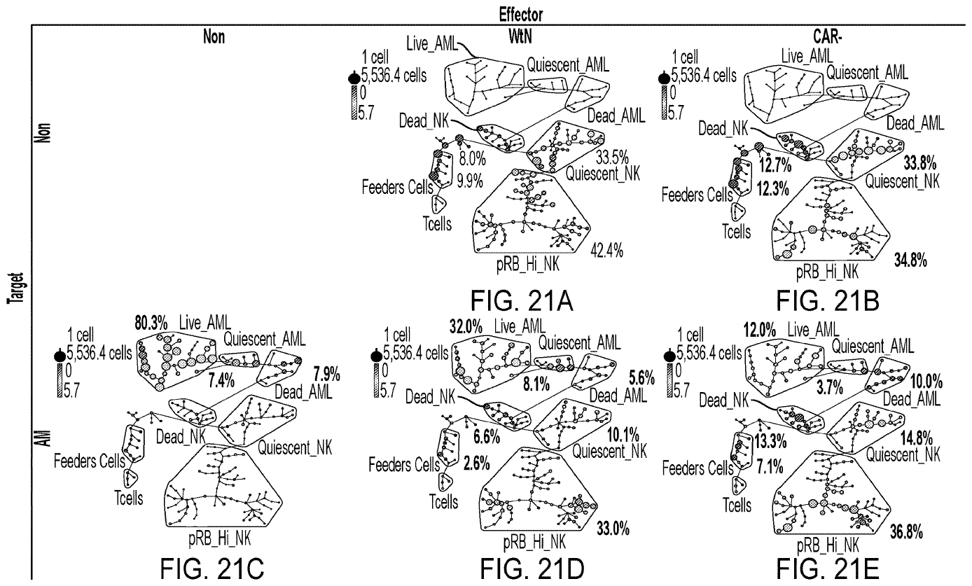

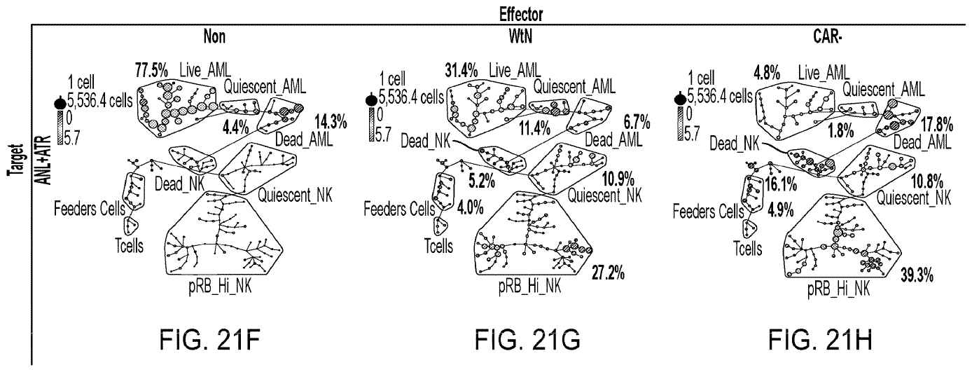

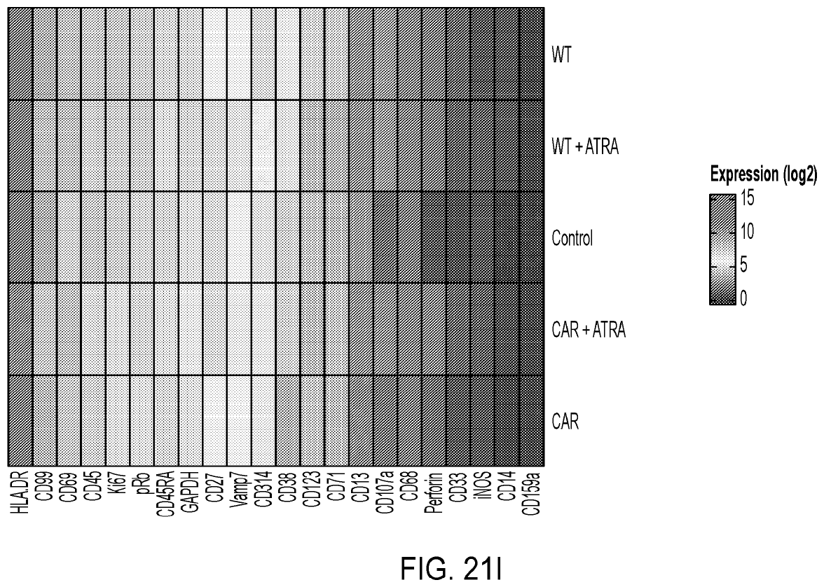

To further augment the antitumor activity of the CD38-CAR NK cells, we explored combination therapy with ATRA. ATRA is a differentiation agent that upregulates CD38 expression in many cell types. Across cell lines of AML, MM, and BL, we observed variable responses in CD38 expression after treatment with 10 nM ATRA for 48 hours (FIG. 20A). The two AML cell lines with the lowest baseline CD38 expression showed the greatest proportional increase in CD38 after ATRA treatment (FIG. 21B). Conversely, intermediate to high CD38-expressing MM cell lines saw a minor increase in expression, while high CD38-expressing BL showed no observable increase (FIG. 20B). A significant increase in tumor cell killing between the CAR NK cells alone and ATRA+CAR NK cells was observed for H929, AML-10, Raji, and T-ALL (FIG. 20C). No increase in killing by the CAR NK cells with the addition of ATRA was seen for MMIS, MV4-11, or Daudi. Thus, ATRA appears to have a cell line dependent effect on the increase in CD38 surface density and sensitivity to CAR NK cell killing (FIG. 20A, 20C). ATRA did not have any adverse effect on the function or viability of CD38^{KO} NK cells.

### ATRA and CD38-CAR NK Cell Combination Treatment Reduced Live Primary AML Cell Populations

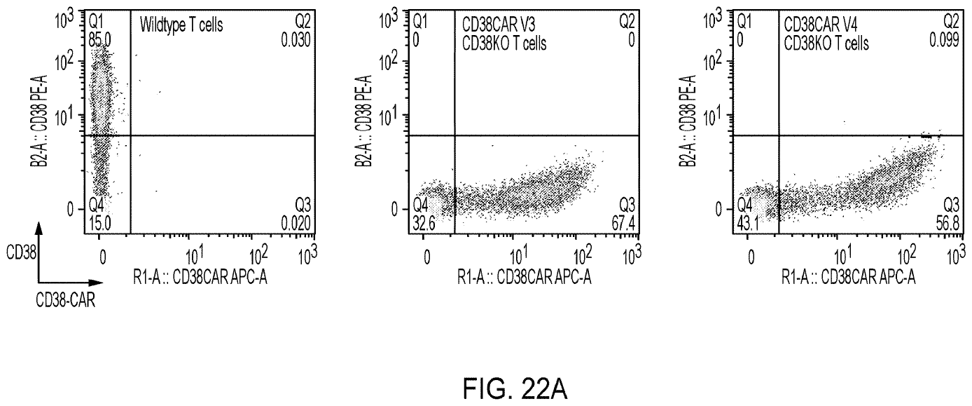

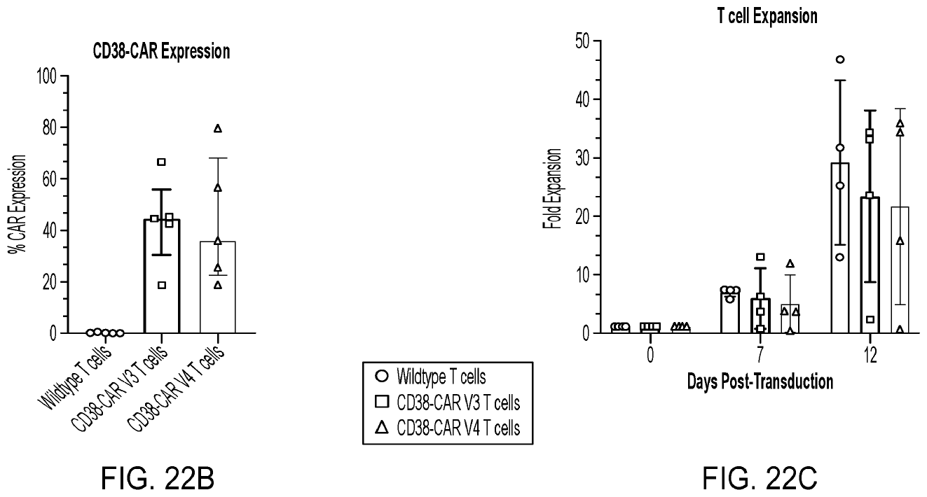

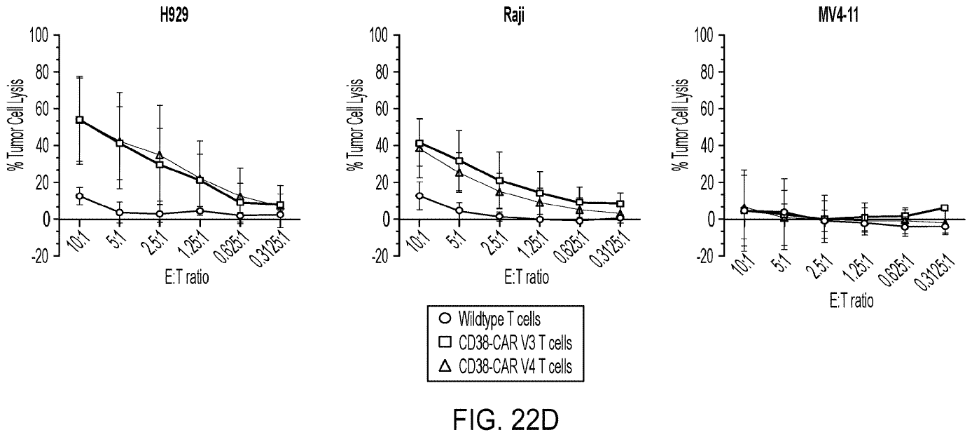

Mass cytometry analysis was performed to further evaluate the efficacy of the combination ATRA and CD38-CAR NK cell treatment. Primary AML cells with and without ATRA prior to co-culture with NK cells had live cell populations of 77.5% and 80.3% respectively (FIG. 21C, 21F). After co-culture with wildtype NK cells with and without ATRA, AML live cell populations decreased to 31.4% and 32.0% respectively (FIG. 21D, 21G). A further reduction was observed when AML cells were treated with CD38-CAR NK cells, showing only 12% remaining live AML (FIG. 22E). The greatest reduction in live AML cell populations, however, was seen when ATRA and CD38-CAR NK cells were used in combination, leaving only 4.8% live AML (FIG. 21H) as a result of upregulation of CD38 (FIG. 21I). We also observed that primary AML co-treated with ATRA and CD38-CAR NK cells had the highest AML dead cell population (17.8%). Notably, there was an increase in live proliferating (pRb+) CD38-CAR NK cells after coculture with AML (FIG. 21E) or AML and ATRA (FIG. 21H), whereas this population decreased after coculture with AML (FIG. 21D) or AML and ATRA (FIG. 21G), indicating that the CAR can also deliver a survival or proliferative signal to the NK cells.

### CD38^{KO}/CD38-CAR is Reproducible and Functional in T Cells

CAR T cells are effective treatments for a variety of hematologic malignancies as an alternative to chemotherapy. Anti-CD38 CAR T cells have recently shown efficacy against MM, T-ALL, and AML in xenografted mouse models. To understand how the cytotoxic function of CAR NK cells compared to CAR T cells, we also generated CD38^{KO}/CD38-CAR T cells using the same CRISPR/Cas9 and adenovirus transduction method. CD38-CAR expression and complete CD38 knockouts were observed in the CAR T cells seven days post-transduction (FIG. 22A). The median adenovirus transduction efficiency for V3 was 44.4% and for V4 was 35.9% (FIG. 22B). Using the same transduction conditions of 75,000 vg/cell of AAV6, this transduction efficiency in T cells was lower than in NK cells (60.7% vs 61.9% for V3 and V4, respectively (FIG. 18C). The CAR T cells expanded the same as wildtype T cells (FIG. 22C), which indicates that the cells avoid fratricide as was observed with CAR NK cells. CAR T cells had enhanced cytotoxicity toward high antigen density tumor cell lines H929 and Raji, but they showed no significant increase in killing of CD38^{low} MV4-11 (FIG. 22D). In this single time-point assay, CAR T cells had lower percent tumor cell lysis than CAR NK cells across all cell lines and E:T ratios, due to lower CAR expression in the T cells or additional innate cytolytic activity of the NK cells. Maximum killing at the 10:1 E:T ratio was 54.1% for H929 and 41.5% for Raji with CAR T cells but was 73.7% for H929 and 84.9% for Raji with the CAR NK cells (FIG. 22D and FIG. 18E).

CD38-CAR NK Cells Avoid Fratricide without CD38^{KO}

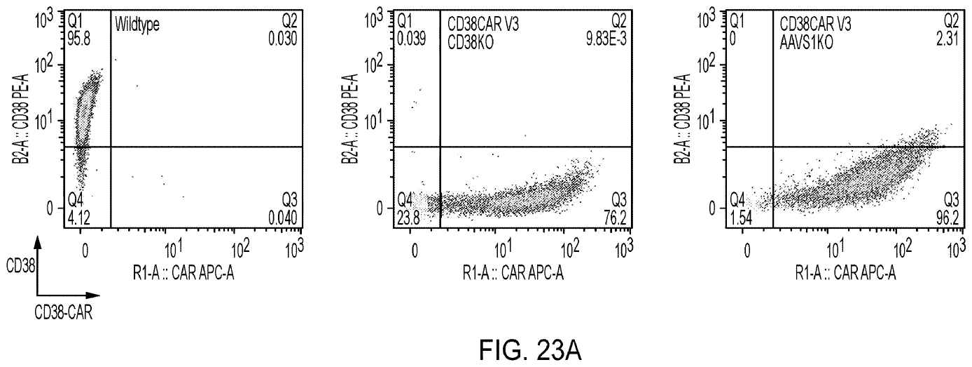

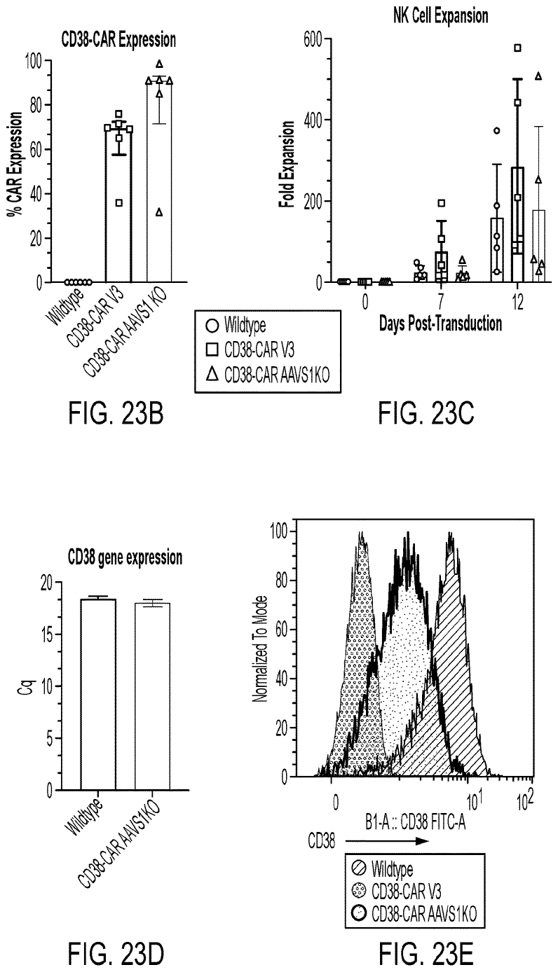

To establish whether CD38 deletion is necessary to avoid fratricide amongst the CAR NK cells, we generated CD38-CAR NK cells using the same CAR constructs but with AAVS1 as the knockout/gene insertion site, leaving the CD38 gene intact. Because the CAR NK cells still express CD38, we hypothesized that they would experience fratricide as is seen for wildtype NK cells in the presence of daratumumab. Instead, the AAVS1^{KO}/CD38-CAR NK cells were 90% CAR-positive seven days after transduction and CD38 was not detectable by flow cytometry (FIG. 23A). The median transduction efficiency for the CD38^{KO}/CD38-CAR NK cells was 69.6% and for the AAVS1^{KO}/CD38-CAR NK cells was 91.2% (FIG. 23B). AAVS1^{KO}/CD38-CAR NK cells expanded like wildtype NK cells, implying no significant fratricide was occurring and CAR NK cells were not dying (FIG. 23C). Because the CD38 gene was left intact for the AAVS1^{KO}/CD38-CAR NK cells but CD38 was undetectable initially, we used reverse transcriptase qPCR to assess CD38 gene expression. Surprisingly, the AAVS1^{KO} CAR NK cells showed equivalent CD38 gene expression to wildtype NK cells (FIG. 23D). To further confirm expression at the protein level, we re-assessed by flow cytometry using a polyclonal anti-CD38 antibody and observed CD38 surface expression (FIG. 23E). The lack of expression using the monoclonal antibody is likely explained by steric hindrance through cis interactions of CD38 with the CAR. The reduced detection by the polyclonal antibody can also be explained by steric inhibition of some, but not all, epitopes recognized by the antibody. The qPCR results show that the CAR has not selected for cells with reduced transcription of CD38, but it is still possible that the CAR selected for cells with some as-yet-unknown epigenetic mechanism that results in reduced protein translation or surface translocation.

### CD38^{KO} in CD38-CAR NK Cells is not Necessary for Increased Cytotoxicity and Metabolism

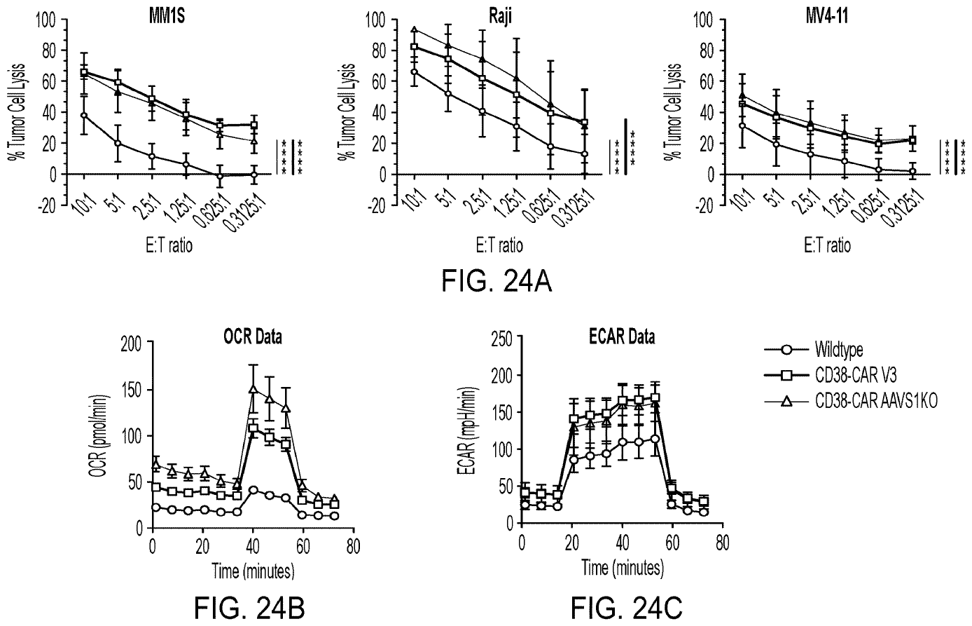

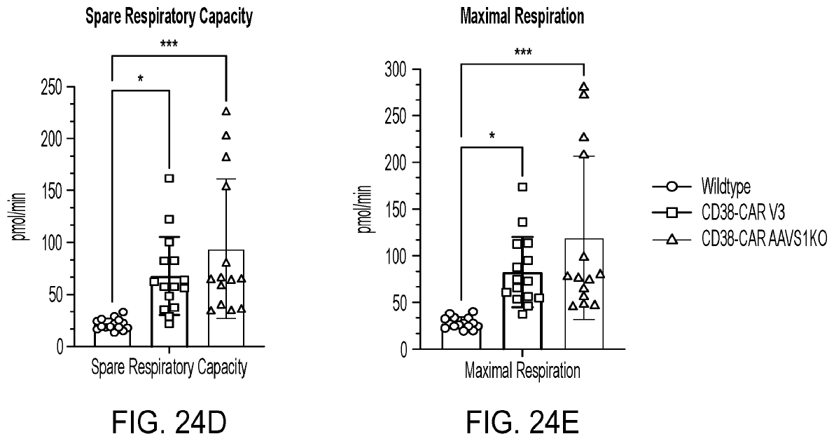

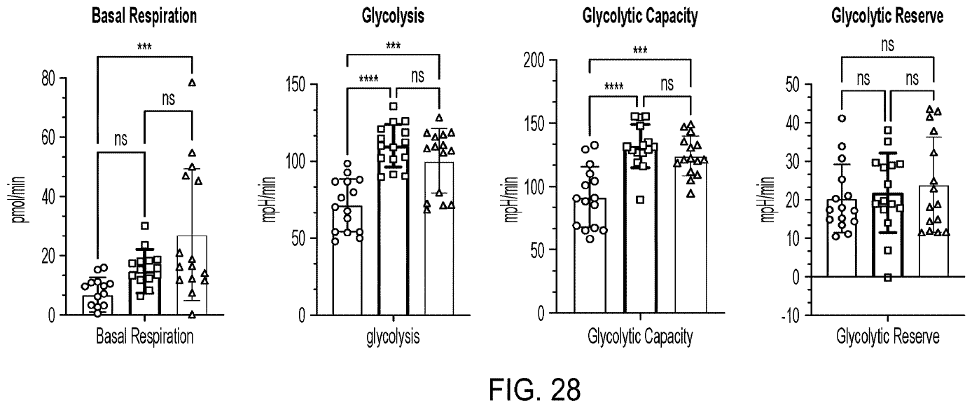

The function of CAR NK cells in both insertion sites (CD38^{KO} and the AAVS1^{KO}) was analyzed by examining cytotoxicity and metabolism in these cells compared to wildtype NK cells. Cytotoxicity assays were performed against MM1S, Raji, and MV4-11. Regardless of the insertion site, both CAR NK cells had significantly higher killing than wildtype NK cells against the CD38+MM, BL, and AML cell lines (FIG. 24A). In addition, both CAR NK cells had significantly higher rates of oxidative phosphorylation than wildtype NK cells (FIG. 24B). The AAVS1^{KO}/CD38-CAR NK cells had higher basal respiration rate and maximal respiration capacity than the CD38^{KO}/CD38-CAR NK cells (FIG. 24B and FIG. 28). Extracellular acidification rates (ECAR) for both CARs, regardless of the insertion site, were higher than wildtype NK cells which indicated that the CAR NK cells perform glycolysis at a faster rate and have a higher overall glycolytic capacity (FIG. 24C). In addition, CD38-CAR NK cells, regardless of the insertion site, had significantly higher spare respiratory capacity than wildtype NK cells (FIG. 24D). Both CAR conditions also displayed significantly higher maximal respiration than wildtype NK cells (FIG. 24E). Overall, this data indicates that CD38 inhibition is present in both of the CD38-CAR NK products, resulting in increased metabolic fitness and flexibility.

### Discussion

Our study reports the successful generation and in vitro evaluation of CD38-CAR NK cells as immunotherapeutics against a broad range of hematological malignancies. We developed two unique CD38-CAR constructs with isatuximab-based scFv, CD3ζ and 41BB co-stimulatory, and a CD8α TM, having reversed heavy and light chain orientations. Both constructs were evaluated for their ability to express the CAR, proliferate without fratricide, and target CD38 expressing malignancies. After determining comparable function between alternative scFv constructs, we proceeded to evaluate the V3 CD38^{KO}/CD38-CAR NK cells for cytolytic activity against primary patient samples and cell lines, cytokine production, and metabolism. We additionally explored ATRA for enhancing the expression of CD38 density on tumor cells and thereby improve CD38-CAR NK cell killing.

MM expresses CD38, and CD38-targeting mAbs (e.g., isatuximab and daratumumab) are effective and well tolerated therapies. There is an unmet need for novel treatments for relapsed/refractory T-cell lymphomas, BL, and AML, which also express CD38. We explored the feasibility of engineering NK cells with an anti-CD38 CAR construct to improve specificity and augment their innate killing potential toward hematologic malignancies. CD38 mAbs induce fratricide among NK cells, but CD38^{KO} NK cells avoided CD38 mAb-mediated fratricide. Here, we explored inserting the isatuximab-based CAR into the CD38 locus in NK cells and thereby create a simultaneous knockout/knockin. The CD38^{KO}/CD38-CAR NK cells are fratricide resistant, proliferate similar to wildtype NK cells, and show enhanced killing toward cell lines and primary patient tumor cells of a wide range of hematologic malignancies. CD38-CAR NK cells displayed increased production of pro-inflammatory cytokines (IFN-γ and TNF-α), GM-CSF, and proinflammatory chemokines (MCP-1, MIP-1α, and RANTES), which are released by activated NK cells to promote immune cell recruitment and maturation.

ATRA is an anti-cancer differentiation agent currently used to treat acute promyelocytic leukemia (APL) and neuroblastoma. ATRA reverses anti-CD38 resistance to daratumumab by upregulating CD38 expression on tumor cells. We explored using ATRA to enhance CD38 expression on AML, MM, BL, and T-ALL to improve sensitivity of tumor cells to CD38-CAR mediated lysis in an isatuximab-based therapy. ATRA induced the greatest increases in surface CD38 expression in immature, CD38-low tumor cell lines, including AML-10, MV4-11, and AML-1. Additionally, primary patient AML cells (AML-10 and AML-1) with lower CD38 surface density displayed a significant increase in cell lysis when co-treated with ATRA and CD38-CAR NK cells. Mass cytometry revealed combination treatments of ATRA and CD38-CAR NK cells against primary patient AML displayed the greatest decreases in live AML populations. The combination therapy also showed a significant decrease in the population of quiescent AML cells which are responsible for the progression and maintenance of the leukemia clone, and therefore elimination of quiescent leukemic cells is vital to eliminating residual disease and preventing relapse. The therapeutic ability of an ATRA and CD38-CAR NK cell combination therapy can be important in targeting CD38-low disease.

The efficacy and application of CD38 targeted CAR T cells therapies for use in AML, MM, and T-ALL, establishing their broad impact on diverse patient populations. CAR T cells therapies can treat hematologic malignancies that are resistant to chemotherapy. Therefore, we developed CD38-CAR T cells using the same CRISPR/Cas9 genome editing and AAV gene delivery approach performed for the CAR NK cells to establish reproducibility of the method in T cells and compare their cytolytic function. While our model was reproducible and functional in T cells, CD38-CAR T cells displayed lower cytotoxicity toward the tumor cell lines AML, MM, and BL across all effector-to-target ratios when compared to CAR NK cells. CD38-CAR T cells also displayed no cytotoxic function against the CD38-low expressing AML cell line MV4-11.

CD38 knockout in NK cells is effective at overcoming fratricide in the presence of CD38 mAbs, we produced a simultaneous CD38^{KO}/CD38-CAR NK cell to avoid CAR-mediated fratricide amongst the NK cells. While the CD38^{KO}/CD38-CAR NK cells model showed fratricide resistance and cytotoxicity toward target cells, we sought to understand whether a CD38^{KO} was necessary for optimal antitumor activity, and used the same approach to generate AAVS1^{KO}/CD38-CAR NK cells. The AAVS1^{KO} cells had unaffected CD38 gene expression and surface expression measured with a polyclonal antibody, but undetected with a monoclonal antibody, which can be explained by steric hinderance through cis interactions masking CD38 epitopes. Sterically blocking detection of CD38 from surrounding cells allows the CAR NK cells to avoid fratricide without a CD38^{KO}. Both CD38^{KO} and AAVS1^{KO} CD38-CAR NK cells showed similarly high increases in killing toward CD38+ hematologic malignancies. Additionally, both CD38-CAR NK cells, regardless of CD38^{KO} or AAVS1^{KO}, had increased metabolic function compared to conventional NK cells, indicating that CD38, even if expressed, is not functional. Unlike daratumumab, isatuximab's binding site mediates inhibition of CD38 metabolic activity, which can also be the mechanism for metabolic enhancement of this isatuximab-derived CAR.

### REFERENCES

Ben Baruch B, Blacher E, Mantsur E, et al. Stromal CD38 regulates outgrowth of primary melanoma and generation of spontaneous metastasis. Oncotarget. 2018; 9(61):31797-31811.
Caligiuri M A. Human natural killer cells. Blood. 2008; 112(3):461-469.
Casneuf T, Xu X S, Adams H C, 3rd, et al. Effects of daratumumab on natural killer cells and impact on clinical outcomes in relapsed or refractory multiple myeloma. Blood Adv. 2017; 1(23):2105-2114.
Denman C J, Senyukov V V, Somanchi S S, et al. Membrane-bound IL-21 promotes sustained ex vivo proliferation of human natural killer cells. PLoS One. 2012; 7(1):e30264.
Fauriat C, Long E O, Ljunggren H G, Bryceson Y T. Regulation of human N K-cell cytokine and chemokine production by target cell recognition. Blood. 2010; 115(11):2167-2176.
Foster C, Kuruvilla J. Treatment approaches in relapsed or refractory peripheral T-cell lymphomas. F1000Res. 2020; 9.
Frampton J E. Isatuximab: A Review of Its Use in Multiple Myeloma. Target Oncol. 2021; 16(5):675-686.
Glisovic-Aplenc T, Diorio C, Chukinas J A, et al. CD38 as a pan-hematologic target for chimeric antigen receptor T cells. Blood Adv. 2023; 7(16):4418-4430.
Graham C, Jozwik A, Pepper A, Benjamin R. Allogeneic CAR-T Cells: More than Ease of Access? Cells. 2018; 7(10).
Gurney M, Stikvoort A, Nolan E, et al. CD38 knockout natural killer cells expressing an affinity optimized CD38 chimeric antigen receptor successfully target acute myeloid leukemia with reduced effector cell fratricide. Haematologica. 2022; 107(2):437-445.
Haslauer T, Greil R, Zaborsky N, Geisberger R. CAR T-Cell Therapy in Hematological Malignancies. Int J Mol Sci. 2021; 22(16).
Kumar S K, Lee J H, Lahuerta J J, et al. Risk of progression and survival in multiple myeloma relapsing after therapy with IMiDs and bortezomib: a multicenter international myeloma working group study. Leukemia. 2012; 26(1):149-157.
Li Y, Sun R. Tumor immunotherapy: New aspects of natural killer cells. Chin J Cancer Res. 2018; 30(2):173-196.
Martin T, Strickland S, Glenn M, et al. Phase I trial of isatuximab monotherapy in the treatment of refractory multiple myeloma. Blood Cancer J. 2019; 9(4):41.
Martin T G, Corzo K, Chiron M, et al. Therapeutic Opportunities with Pharmacological Inhibition of CD38 with Isatuximab. Cells. 2019; 8(12).
Martino M, Alati C, Canale F A, Musuraca G, Martinelli G, Cerchione C. A Review of Clinical Outcomes of CAR T-Cell Therapies for B-Acute Lymphoblastic Leukemia. Int J Mol Sci. 2021; 22(4).
Naeimi Kararoudi M, Likhite S, Elmas E, et al. Optimization and validation of CAR transduction into human primary N K cells using CRISPR and AAV. Cell Rep Methods. 2022; 2(6):100236.
Naeimi Kararoudi M, Nagai Y, Elmas E, et al. CD38 deletion of human primary N K cells eliminates daratumumab-induced fratricide and boosts their effector activity. Blood. 2020; 136(21):2416-2427.
Newell L F, Cook R J. Advances in acute myeloid leukemia. BMJ. 2021; 375:n2026.
Nijhof I S, Casneuf T, van Velzen J, et al. CD38 expression and complement inhibitors affect response and resistance to daratumumab therapy in myeloma. Blood. 2016; 128(7):959-970.
Nijhof I S, Groen R W, Lokhorst H M, et al. Upregulation of CD38 expression on multiple myeloma cells by all-trans retinoic acid improves the efficacy of daratumumab. Leukemia. 2015; 29(10):2039-2049.
Passarelli A, Tucci M, Mannavola F, Felici C, Silvestris F. The metabolic milieu in melanoma: Role of immune suppression by CD73/adenosine. Tumour Biol. 2019; 42(4):1010428319837138.
Rong Q X, Wang F, Guo Z X, et al. G M-CSF mediates immune evasion via upregulation of PD-L1 expression in extranodal natural killer/T cell lymphoma. Mol Cancer. 2021; 20(1):80.
Saito Y, Uchida N, Tanaka S, et al. Induction of cell cycle entry eliminates human leukemia stem cells in a mouse model of AML. Nat Biotechnol. 2010; 28(3):275-280.
Sanchez L, Wang Y, Siegel D S, Wang M L. Daratumumab: a first-in-class CD38 monoclonal antibody for the treatment of multiple myeloma. J Hematol Oncol. 2016; 9(1):51.
Schuh W, Mielenz D, Jack H M. Unraveling the mysteries of plasma cells. Adv Immunol. 2020; 146:57-107.
Short N J, Kantarjian H M, Ko H, et al. Outcomes of adults with relapsed or refractory Burkitt and high-grade B-cell leukemia/lymphoma. Am J Hematol. 2017; 92(6):E114-E117.
Siegler E L, Zhu Y, Wang P, Yang L. Off-the-Shelf CAR-NK Cells for Cancer Immunotherapy. Cell Stem Cell. 2018; 23(2):160-161.
Somanchi S S, Lee D A. Ex Vivo Expansion of Human N K Cells Using K562 Engineered to Express Membrane Bound IL21. Methods Mol Biol. 2016; 1441:175-193.
Somanchi S S, Senyukov V V, Denman C J, Lee D A. Expansion, purification, and functional assessment of human peripheral blood N K cells. J Vis Exp. 2011(48).
Sterner R C, Sterner R M. CAR-T cell therapy: current limitations and potential strategies. Blood Cancer J. 2021; 11(4):69.
van de Donk N, Usmani S Z. CD38 Antibodies in Multiple Myeloma: Mechanisms of Action and Modes of Resistance. Front Immunol. 2018; 9:2134.
Wang Y, Zhang Y, Hughes T, et al. Fratricide of N K Cells in Daratumumab Therapy for Multiple Myeloma Overcome by Ex Vivo-Expanded Autologous N K Cells. Clin Cancer Res. 2018; 24(16):4006-4017.
Young R M, Engel N W, Uslu U, Wellhausen N, June C H. Next-Generation CAR T-cell Therapies. Cancer Discov. 2022; 12(7):1625-1633.

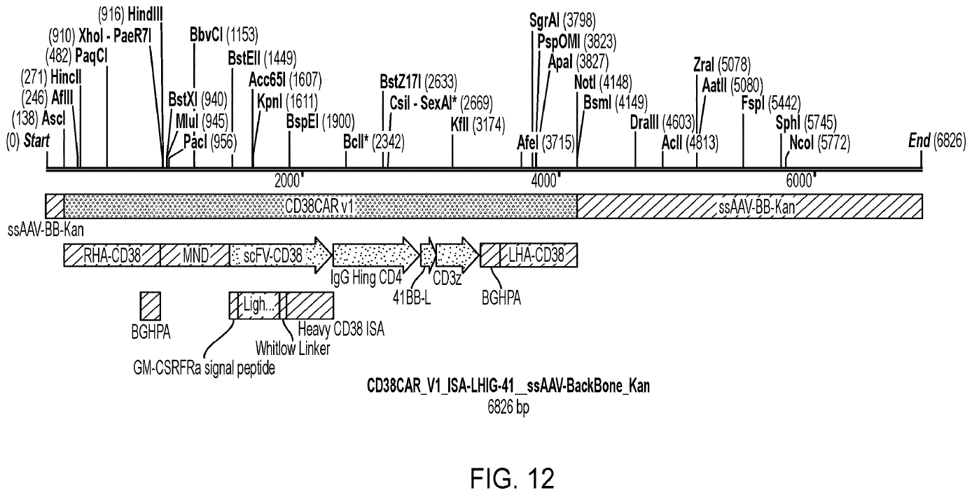

SEQUENCES
gRNA sequence
SEQ ID NO: 1
ctgaactcgcagttggccat
LEFT CD38 HOMOLOGY
SEQ ID NO: 2
GAAGTACAGAGAGGTTAAGTAACTTGCCCCATGTCATCCAGCAAGAACTAAATT
TGAACCCAGAGCTTAGCCACTGATGCCTCTTGAGAGAAGGAGTCAGACTTAAGT
TGAGTCTTTAAAGGTGGTTGACCAGGCATTTGTCAGAGTTAAGAAAGAGAGGTA
GGACATCCTTTTCCAGGCAGAGGGCATTGTGTGCACACACGTATAGAAGCAGGC
AGCCCACCCTCATGCTTTCCAGGAAGCAAATGTGGCTCAGGTGTAAAGTGCCCG
GTTGATGAAGGGAGTTAGCGGAGGGAGTATAAGGATGTACTGTCTGCCCCCTTA
GGACACCTGCAGAGGATTAAGGTGGCTGTTTCTCCCTGGAGGTGGAGTGGGTGG
GTCACTGCACAGGAGCCTATAGTTGTTGGTCTTTTAAACTCTTATTGGTGTAACC
AGCCACGGAACTCTGAGGCAAGGGGTTGGGGGTGGGAAGGGAAACAGAGAAAA
GGCAAGTGAAACAGAAGGGGAGGTGCAGTTTCAGAACCCAGCCAGCCTCTCTCT
TGCTGCCTAGCCTCCTGCCGGCCTCATCTTCGCCCAGCCAACCCCGCCTGGAGCC
CTATG
RIGHT CD38 HOMOLOGY ARM:
SEQ ID NO: 3
GCCAACTGCGAGTTCAGCCCGGTGTCCGGGGACAAACCCTGCTGCCGGCTCTCT
AGGAGAGCCCAACTCTGTCTTGGCGTCAGTATCCTGGTCCTGATCCTCGTCGTGG
TGCTCGCGGTGGTCGTCCCGAGGTGGCGCCAGCAGTGGAGCGGTCCGGGCACCA
CCAAGCGCTTTCCCGAGACCGTCCTGGCGCGATGCGTCAAGTACACTGAAATTC
ATCCTGAGATGAGGTGGGTTGGCGACTAAGGCGCACCGGTGGGCACTGCGGGGA
CAGCAGGGCCCCGCGCGCAGGGAAGCCGCCCGGATCGCCCGGAACCGGGCATCT
TCCGTGGCGGGTCAGCCGAGAGCCCGCCGGGTGGTGCTGAGTAGGGAGTCCCGG
GCTCGGGGCTCCGCGGGCCGCTTTCAGGAGCAGCTGGCCTTGGCACCGAGCGTG
CCCGCGGGAGGCGGGGGGGGGCGCTGCTCGGTGGCTCTGCTGCGTAGCCGGTGA
ACACTTGGCACCGATGCCCGCCTTCTGGGCAAGGTGCCCTGAGCCCAGCCCCTCG
CCGGGCTGCAGCCCACCCTCGGCGCGCTCAGCCCGCTTCACCGCTTCAGGGACG
GAAT
BGH polyA terminator
SEQ ID NO: 4
cctcgactgtgccttctagttgccagccatctgttgtttgcccctcccccgtgccttccttgaccctggaaggtgccactcccactgtccttt
cctaataaaatgaggaaattgcatcgcattgtctgagtaggtgtcattctattc
(crRNA)
SEQ ID NO: 5
GGGGCCACTAGGGACAGGAT
GM-CSRFRa signal peptide:
SEQ ID NO: 6
MLLLVTSLLLCELPHPAFLLIP
CD28:
SEQ ID NO: 7
Atgttttgggtgctggtggtggtgggaggcgtgctggcctgttattccctgctggtgaccgtggccttcatcatcttttgggtgcgctcca
agcggagccggggcggacactctgactacatgaacatgaccccacggagacccggacctacaaggaagcactatcagccctacg
cccctccacgggacttcgcagcatatcgcagc
CD3z:
SEQ ID NO: 8
Cgggtgaagtttagcagatccgccgatgcaccagcatatcagcagggacagaatcagctgtacaacgagctgaatctgggcaggc
gcgaggagtacgacgtgctggataagaggcggggccgggaccccgagatgggaggcaagcccaggcgcaagaaccctcagga
gggcctgtataatgagctgcagaaggacaagatggccgaggcctacagcgagatcggcatgaagggagagcggagaaggggca
agggacacgatggcctgtatcagggcctgtccaccgccacaaaggacacctacgatgcactgcacatgcaggccctgccacctcgg
tga
Whitlow linker
SEQ ID NO: 9
GSTSGSGKPGSGEGSTKG
CD8 stalk:
SEQ ID NO: 10
TTTPAPRPPTPAPTIASQPLSLRPEACRPAAGGAVHTRGLDFACDIYIWAPLAGTCGV
LLLSLVITLYC
NKG2D Transmembrane domain:
SEQ ID NO: 11
Agcaacctgttcgtggcctcctggatcgccgtgatgatcatctttcgcatcggcatggccgtggccatcttctgctgtttctttttcccatc
c
Linker:
SEQ ID NO: 12
Ggaggctctggaggaggctccggc
2B4:
SEQ ID NO: 13
Tggcggagaaagcggaaggagaagcagagcgagacctcccctaaggagtttctgacaatctatgaggacgtgaaggatctgaag
accaggcgcaatcacgagcaggagcagaccttcccaggaggaggctctacaatctacagcatgatccagtcccagagcagcgccc
caaccagccaggagccagcctatacactgtactctctgatccagcctagccggaagtctggcagccgcaageggaaccactcccca
tctttcaattctaccatctatgaagtgatcggcaagagccagcctaaggcccagaacccagccagactgtccaggaaggagctggag
aattttgacgtgtactct
Linker:
SEQ ID NO: 14
Ggaggcagcggaggaggctctggc
CD3z:
SEQ ID NO: 15
Cgcgtgaagttcagccggtccgccgatgccccagcctataagcagggccagaaccagctgtacaacgagctgaatctgggccgga
gagaggagtacgacgtgctggataagaggcggggccgggaccccgagatgggaggcaagccccggagaaagaaccctcagga
gggcctgtataatgagctgcagaaggacaagatggccgaggcctactccgagatcggcatgaagggagagaggcgccggggcaa
gggacacgatggcctgtatcagggcctgagcaccgccacaaaggacacctacgatgccctgcacatgcaggccctgcctccacgg
tgatga
CD38CAR_V4_ISA-HLCD8-41 ssAAV-BackBone_KanAAAE46
SEQ ID NO: 16
CCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCTGCGGCCGGCGCGCCGAAGTACAGAGAGGTTAA
GTAACTTGCCCCATGTCATCCAGCAAGAACTAAATTTGAACCCAGAGCTTAGCC
ACTGATGCCTCTTGAGAGAAGGAGTCAGACTTAAGTTGAGTCTTTAAAGGTGGTT
GACCAGGCATTTGTCAGAGTTAAGAAAGAGAGGTAGGACATCCTTTTCCAGGCA
GAGGGCATTGTGTGCACACACGTATAGAAGCAGGCAGCCCACCCTCATGCTTTC
CAGGAAGCAAATGTGGCTCAGGTGTAAAGTGCCCGGTTGATGAAGGGAGTTAGC
GGAGGGAGTATAAGGATGTACTGTCTGCCCCCTTAGGACACCTGCAGAGGATTA
AGGTGGCTGTTTCTCCCTGGAGGTGGAGTGGGTGGGTCACTGCACAGGAGCCTA
TAGTTGTTGGTCTTTTAAACTCTTATTGGTGTAACCAGCCACGGAACTCTGAGGC
AAGGGGTTGGGGGTGGGAAGGGAAACAGAGAAAAGGCAAGTGAAACAGAAGG
GGAGGTGCAGTTTCAGAACCCAGCCAGCCTCTCTCTTGCTGCCTAGCCTCCTGCC
GGCCTCATCTTCGCCCAGCCAACCCCGCCTGGAGCCCTATGCCTCGACTGTGCCT
TCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGT
CTGAGTAGGTGTCATTCTATTCATCGATCACGAGACTAGCCTCGAGAAGCTTGAT
ATCGAATTCCACGGGGTTGGACGCGTCTTAATTAAGGATCCAAGGTCAGGAACA
GAGAAACAGGAGAATATGGGCCAAACAGGATATCTGTGGTAAGCAGTTCCTGCC
CCGGCTCAGGGCCAAGAACAGTTGGAACAGCAGAATATGGGCCAAACAGGATA
TCTGTGGTAAGCAGTTCCTGCCCCGGCTCAGGGCCAAGAACAGATGGTCCCCAG
ATGCGGTCCCGCCCTCAGCAGTTTCTAGAGAACCATCAGATGTTTCCAGGGTGCC
CCAAGGACCTGAAATGACCCTGTGCCTTATTTGAACTAACCAATCAGTTCGCTTC
TCGCTTCTGTTCGCGCGCTTCTGCTCCCCGAGCTCTATATAAGCAGAGCTCGTTTA
GTGAACCGTCAGATCGCCTGGAGACGCCATCCACGCTGTTTTGACCTCCATAGAA
GACACCGACTCTAGAGGATCGATCCCCCGGGCTGCAGGAATTCAAGCGAGAAGA
CAAGGGCAGAAAGCACCGCCACCATGCTGCTGCTGGTGACCTCTCTGCTGCTGT
GCGAGCTGCCACACCCAGCCTTCCTGCTGATCCCACAGGTGCAGCTGCAGCAGT
CCGGAGCAGAGCTGGCCCGGCCCGGCACCTCCGTGAAGCTGTCTTGCAAGGCCA
GCGGCTACACCTTCACAGACTATTGGATGCAGTGGGTGAAGCAGAGACCAGGAC
AGGGACTGGAGTGGATCGGAACCATCTACCCTGGCGACGGCGATACAGGCTATG
CCCAGAAGTTTAAGGGCAAGGCCACCCTGACAGCCGACAAGAGCTCCAAGACA
GTGTACATGCACCTGTCTAGCCTGGCCTCTGAGGATAGCGCCGTGTACTATTGTG
CCCGGGGCGACTACTATGGCAGCAACTCCCTGGATTATTGGGGCCAGGGCACCT
CTGTGACAGTGTCCTCTGGCTCCACCTCTGGCAGCGGCAAGCCTGGCAGCGGAG
AGGGCTCCACAAAGGGAGACATCGTGATGGCCCAGTCCCACAAGTTCATGTCTA
CCAGCGTGGGCGACAGGGTGTCTATCACATGCAAGGCCTCTCAGGATGTGAGCA
CCGTGGTGGCCTGGTACCAGCAGAAGCCAGGCCAGAGCCCCAAGCGCCTGATCT
ATTCCGCCTCTTACCGGTATATCGGAGTGCCAGACAGATTCACCGGAAGCGGAT
CCGGCACAGATTTCACCTTTACAATCAGCTCCGTGCAGGCCGAGGATCTGGCCGT
GTACTATTGTCAGCAGCACTACTCCCCCCCTTATACCTTTGGCGGCGGCACAAAG
CTGGAGATCAAGCGGACAACAACTCCAGCACCCCGCCCACCTACTCCCGCTCCT
ACAATCGCCTCTCAGCCCCTGTCCCTGCGCCCAGAAGCCTGTCGCCCCGCAGCAG
GAGGAGCAGTGCACACCAGGGGCCTGGACTTCGCCTGCGATATCTACATCTGGG
CTCCTCTGGCTGGAACTTGTGGCGTGCTGCTGCTGTCACTGGTCATTACCCTGTAT
TGTAAGCGGGGCAGAAAGAAGCTGCTGTACATCTTCAAGCAGCCCTTTATGAGG
CCTGTGCAGACCACACAGGAGGAGGACGGCTGCAGCTGTCGGTTCCCCGAGGAG
GAGGAGGGCGGCTGCGAGCTGCGGGTGAAGTTTAGCAGATCCGCCGATGCACCA
GCATATCAGCAGGGACAGAATCAGCTGTACAACGAGCTGAATCTGGGCAGGCGC
GAGGAGTACGACGTGCTGGATAAGAGGCGGGGCCGGGACCCCGAGATGGGAGG
CAAGCCCAGGCGCAAGAACCCTCAGGAGGGCCTGTATAATGAGCTGCAGAAGG
ACAAGATGGCCGAGGCCTACAGCGAGATCGGCATGAAGGGAGAGCGGAGAAGG
GGCAAGGGACACGATGGCCTGTATCAGGGCCTGTCCACCGCCACAAAGGACACC
TACGATGCACTGCACATGCAGGCCCTGCCACCTCGGTGAAAGTAACGCCCTCGA
CTGTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGCAT
CGCATTGTCTGAGTAGGTGTCATTCTATTCGCCAACTGCGAGTTCAGCCCGGTGT
CCGGGGACAAACCCTGCTGCCGGCTCTCTAGGAGAGCCCAACTCTGTCTTGGCGT
CAGTATCCTGGTCCTGATCCTCGTCGTGGTGCTCGCGGTGGTCGTCCCGAGGTGG
CGCCAGCAGTGGAGCGGTCCGGGCACCACCAAGCGCTTTCCCGAGACCGTCCTG
GCGCGATGCGTCAAGTACACTGAAATTCATCCTGAGATGAGGTGGGTTGGCGAC
TAAGGCGCACCGGTGGGCACTGCGGGGACAGCAGGGCCCCGCGCGCAGGGAAG
CCGCCCGGATCGCCCGGAACCGGGCATCTTCCGTGGCGGGTCAGCCGAGAGCCC
GCCGGGTGGTGCTGAGTAGGGAGTCCCGGGCTCGGGGCTCCGCGGGCCGCTTTC
AGGAGCAGCTGGCCTTGGCACCGAGCGTGCCCGCGGGAGGCGGGGGGGGGCGC
TGCTCGGTGGCTCTGCTGCGTAGCCGGTGAACACTTGGCACCGATGCCCGCCTTC
TGGGCAAGGTGCCCTGAGCCCAGCCCCTCGCCGGGCTGCAGCCCACCCTCGGCG
CGCTCAGCCCGCTTCACCGCTTCAGGGACGGAATGCGGCCGCAGGAACCCCTAG
TGATGGAGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCGAGCG
AGCGCGCAGCTGCCTGCAGGGGCGCCTGATGCGGTATTTTCTCCTTACGCATCTG
TGCGGTATTTCACACCGCATACGTCAAAGCAACCATAGTACGCGCCCTGTAGCG
GCGCATTAAGCGCGGCGGGTGTGGTGGTTACGCGCAGCGTGACCGCTACACTTG
CCAGCGCCTTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTA
GTGCTTTACGGCACCTCGACCCCAAAAAACTTGATTTGGGTGATGGTTCACGTAG
TGGGCCATCGCCCTGATAGACGGTTTTTCGCCCTTTGACGTTGGAGTCCACGTTC
TTTAATAGTGGACTCTTGTTCCAAACTGGAACAACACTCAACTCTATCTCGGGCT
ATTCTTTTGATTTATAAGGGATTTTGCCGATTTCGGTCTATTGGTTAAAAAATGAG
CTGATTTAACAAAAATTTAACGCGAATTTTAACAAAATATTAACGTTTACAATTT
TATGGTGCACTCTCAGTACAATCTGCTCTGATGCCGCATAGTTAAGCCAGCCCCG
ACACCCGCCAACACCCGCTGACGCGCCCTGACGGGCTTGTCTGCTCCCGGCATCC
GCTTACAGACAAGCTGTGACCGTCTCCGGGAGCTGCATGTGTCAGAGGTTTTCAC
CGTCATCACCGAAACGCGCGAGACGAAAGGGCCTCGTGATACGCCTATTTTTAT
AGGTTAATGTCATGATAATAATGGTTTCTTAGACGTCAGGTGGCACTTTTCGGGG
AAATGTGCGCGGAACCCCTATTTGTTTATTTTTCTAAATACATTCAAATATGTATC
CGCTCATGAGACAATAACCCTGATAAATGCTTCAATAATATTGAAAAAGGAAGA
GTatgattgaacaagatggattgcacgcaggttctccggccgcttgggtggagaggctattcggctatgactgggcacaacagaca
atcggctgctctgatgccgccgtgttccggctgtcagcgcaggggcgcccggttctttttgtcaagaccgacctgtccggtgccctgaa
tgaactgcaggacgaggcagcgcggctatcgtggctggccacgacgggcgttccttgcgcagctgtgctcgacgttgtcactgaag
cgggaagggactggctgctattgggcgaagtgccggggcaggatctcctgtcatctcaccttgctcctgccgagaaagtatccatcat
ggctgatgcaatgcggcggctgcatacgcttgatccggctacctgcccattcgaccaccaagcgaaacatcgcatcgagcgagcac
gtactcggatggaagccggtcttgtcgatcaggatgatctggacgaagagcatcaggggctcgcgccagccgaactgttcgccagg
ctcaaggegcgcatgcccgacggcgaggatctcgtcgtgacccatggcgatgcctgcttgccgaatatcatggtggaaaatggccg
cttttctggattcatcgactgtggccggctgggtgtggcggaccgctatcaggacatagcgttggctacccgtgatattgctgaagagct
tggcggcgaatgggctgaccgcttcctcgtgctttacggtatcgccgctcccgattegcagcgcategccttctatcgccttcttgacga
gttcttctgaCTGTCAGACCAAGTTTACTCATATATACTTTAGATTGATTTAAAACTTCA
TTTTTAATTTAAAAGGATCTAGGTGAAGATCCTTTTTGATAATCTCATGACCAAA
ATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAA
AAAACCACCGCTACCAGCGGTGGTTTGTTTGCCGGATCAAGAGCTACCAACTCTT
TTTCCGAAGGTAACTGGCTTCAGCAGAGCGCAGATACCAAATACTGTTCTTCTAG
TGTAGCCGTAGTTAGGCCACCACTTCAAGAACTCTGTAGCACCGCCTACATACCT
CGCTCTGCTAATCCTGTTACCAGTGGCTGCTGCCAGTGGCGATAAGTCGTGTCTT
ACCGGGTTGGACTCAAGACGATAGTTACCGGATAAGGCGCAGCGGTCGGGCTGA
ACGGGGGGTTCGTGCACACAGCCCAGCTTGGAGCGAACGACCTACACCGAACTG
AGATACCTACAGCGTGAGCTATGAGAAAGCGCCACGCTTCCCGAAGGGAGAAA
GGCGGACAGGTATCCGGTAAGCGGCAGGGTCGGAACAGGAGAGCGCACGAGGG
AGCTTCCAGGGGGAAACGCCTGGTATCTTTATAGTCCTGTCGGGTTTCGCCACCT
CTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCA
CATGT
MND promoter
SEQ ID NO: 17
atcgatcacgagactagcctcgagaagcttgatatcgaattccacggggttggacgcgtcttaattaaggatccaaggtcaggaacag
agaaacaggagaatatgggccaaacaggatatctgtggtaagcagttcctgccccggctcagggccaagaacagttggaacagcag
aatatgggccaaacaggatatctgtggtaagcagttcctgccccggctcagggccaagaacagatggtccccagatgcggtcccgc
cctcagcagtttctagagaaccatcagatgtttccagggtgccccaaggacctgaaatgaccctgtgccttatttgaactaaccaatcag
ttcgcttctcgcttctgttcgcgcgcttctgctccccgagctctatataagcagagctcgtttagtgaaccgtcagatcgcctggagacgc
catccacgctgttttgacctccatagaagacaccgactctagaggatcgatcccccgggctgcaggaattcaagcgagaagacaagg
gcagaaagcacc
anti-CD38 ScFv v1 CD38CAR_V1_ISA-LHIG-41 ssAAV-BackBone_Kan (FIG. 12)
SEQ ID NO: 18

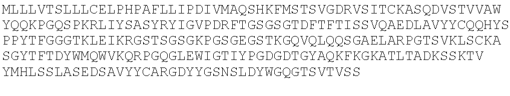

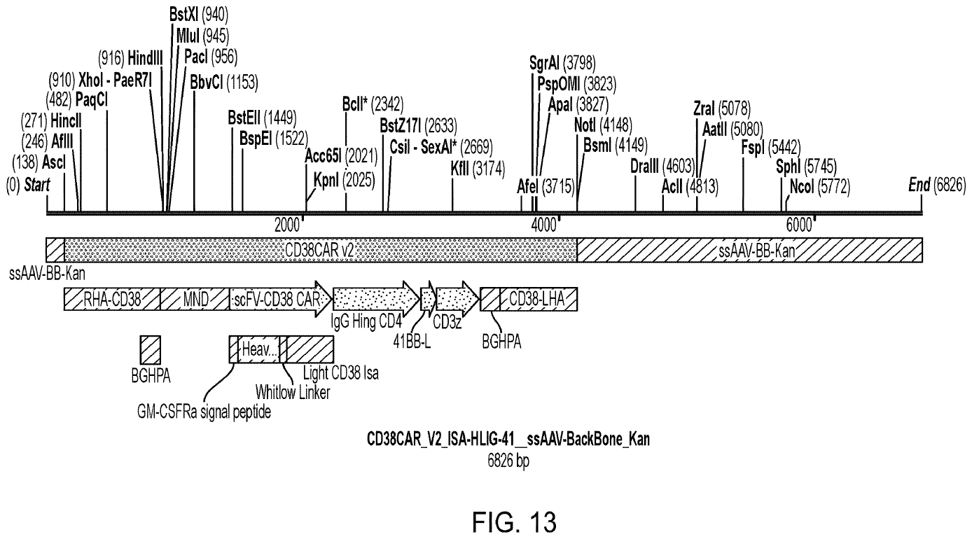

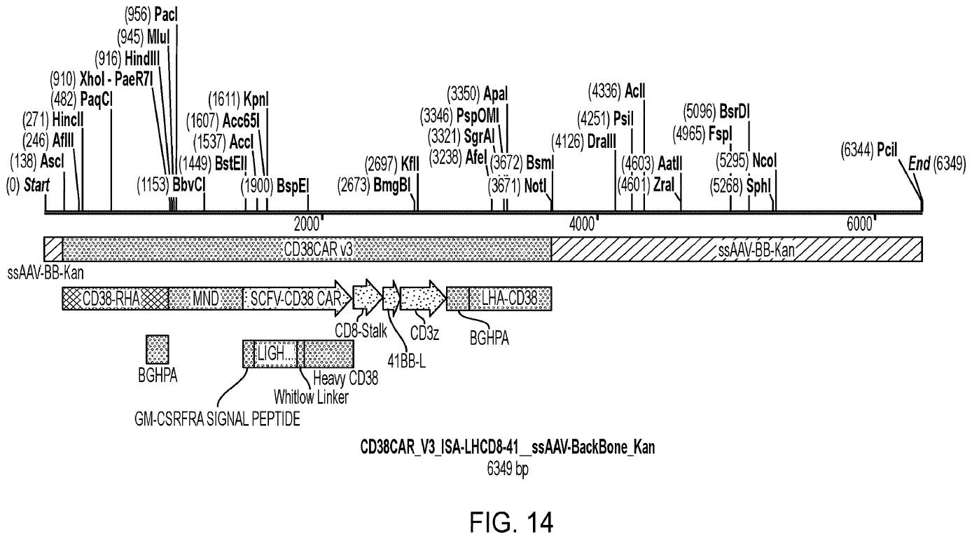

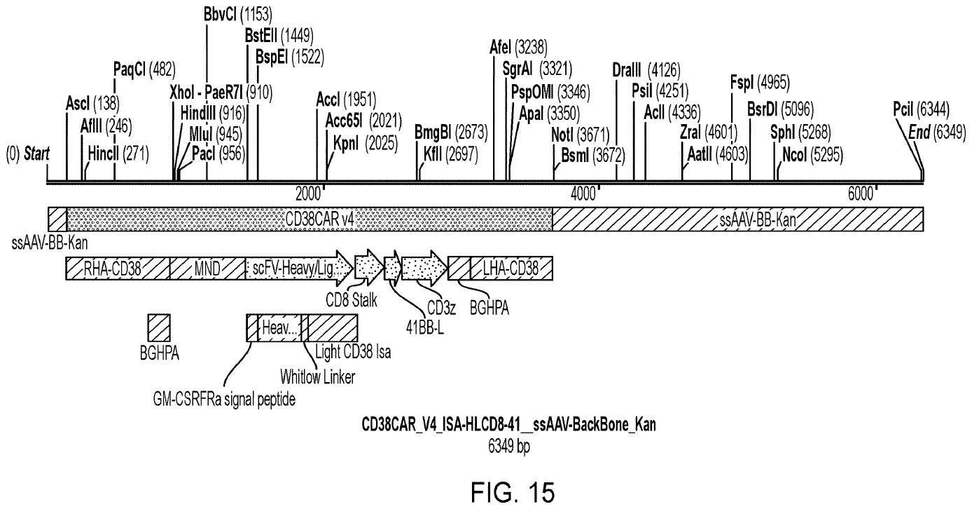

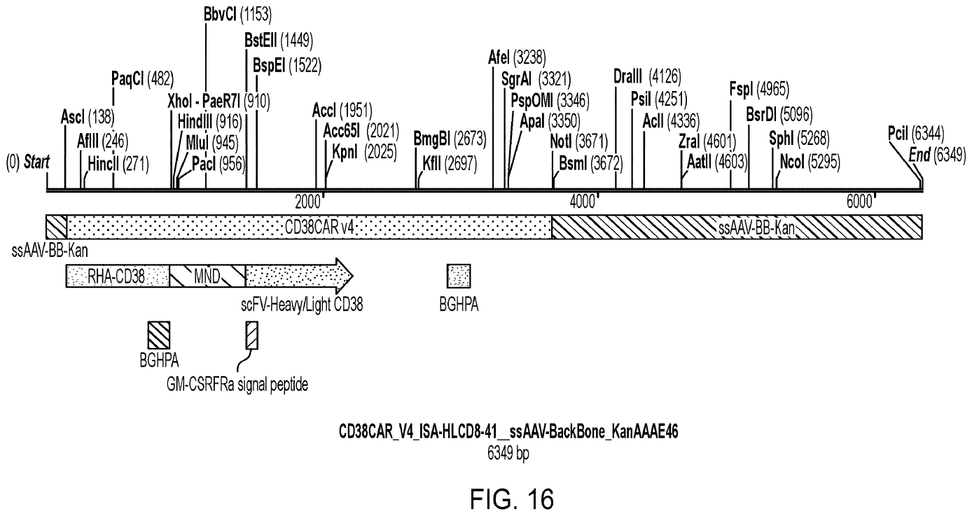

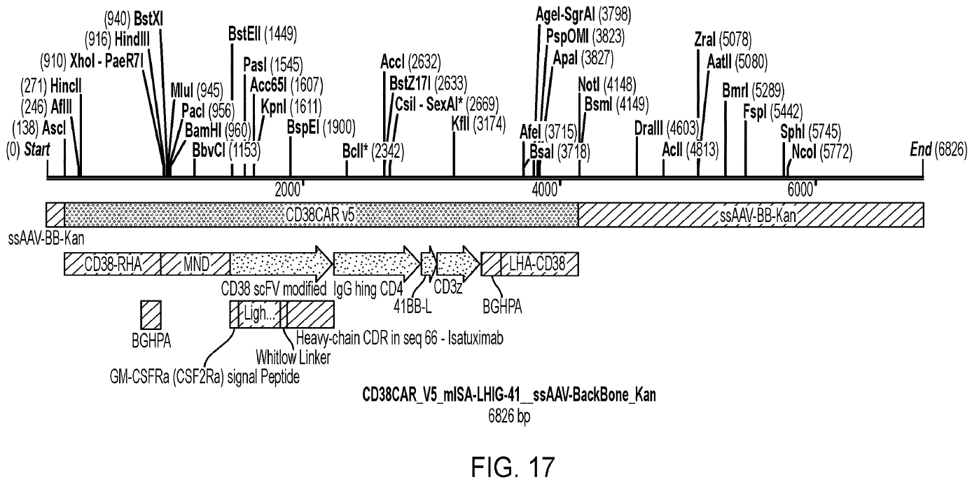

IgG Hinge CD4
SEQ ID NO: 19
ESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSS
IEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSL
G
41BB-L
SEQ ID NO: 20
KRGRKKLLYIFKQPFMRPVQTTQEEDGCSCRFPEEEEGGCEL
CD3z
SEQ ID NO: 21
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
right homology arm
SEQ ID NO: 22
GAAGTACAGAGAGGTTAAGTAACTTGCCCCATGTCATCCAGCAAGAACTAAATT
TGAACCCAGAGCTTAGCCACTGATGCCTCTTGAGAGAAGGAGTCAGACTTAAGT
TGAGTCTTTAAAGGTGGTTGACCAGGCATTTGTCAGAGTTAAGAAAGAGAGGTA
GGACATCCTTTTCCAGGCAGAGGGCATTGTGTGCACACACGTATAGAAGCAGGC
AGCCCACCCTCATGCTTTCCAGGAAGCAAATGTGGCTCAGGTGTAAAGTGCCCG
GTTGATGAAGGGAGTTAGCGGAGGGAGTATAAGGATGTACTGTCTGCCCCCTTA
GGACACCTGCAGAGGATTAAGGTGGCTGTTTCTCCCTGGAGGTGGAGTGGGTGG
GTCACTGCACAGGAGCCTATAGTTGTTGGTCTTTTAAACTCTTATTGGTGTAACC
AGCCACGGAACTCTGAGGCAAGGGGTTGGGGGTGGGAAGGGAAACAGAGAAAA
GGCAAGTGAAACAGAAGGGGAGGTGCAGTTTCAGAACCCAGCCAGCCTCTCTCT
TGCTGCCTAGCCTCCTGCCGGCCTCATCTTCGCCCAGCCAACCCCGCCTGGAGCC
CTATGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGA
GGAAATTGCATCGCATTGTCTGAGTAGGTGTCATTCTATTC
left homology arm
SEQ ID NO: 23
GCCAACTGCGAGTTCAGCCCGGTGTCCGGGGACAAACCCTGCTGCCGGCTCTCT
AGGAGAGCCCAACTCTGTCTTGGCGTCAGTATCCTGGTCCTGATCCTCGTCGTGG
TGCTCGCGGTGGTCGTCCCGAGGTGGCGCCAGCAGTGGAGCGGTCCGGGCACCA
CCAAGCGCTTTCCCGAGACCGTCCTGGCGCGATGCGTCAAGTACACTGAAATTC
ATCCTGAGATGAGGTGGGTTGGCGACTAAGGCGCACCGGTGGGCACTGCGGGGA
CAGCAGGGCCCCGCGCGCAGGGAAGCCGCCCGGATCGCCCGGAACCGGGCATCT
TCCGTGGCGGGTCAGCCGAGAGCCCGCCGGGTGGTGCTGAGTAGGGAGTCCCGG
GCTCGGGGCTCCGCGGGCCGCTTTCAGGAGCAGCTGGCCTTGGCACCGAGCGTG
CCCGCGGGAGGCGGGGGGGGGCGCTGCTCGGTGGCTCTGCTGCGTAGCCGGTGA
ACACTTGGCACCGATGCCCGCCTTCTGGGCAAGGTGCCCTGAGCCCAGCCCCTCG
CCGGGCTGCAGCCCACCCTCGGCGCGCTCAGCCCGCTTCACCGCTTCAGGGACG
GAAT
CD38CAR_V1_ISA-LHIG-41 ssAAV-BackBone_Kan
SEQ ID NO: 24
CCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCTGCGGCCGGCGCGCCGAAGTACAGAGAGGTTAA
GTAACTTGCCCCATGTCATCCAGCAAGAACTAAATTTGAACCCAGAGCTTAGCC
ACTGATGCCTCTTGAGAGAAGGAGTCAGACTTAAGTTGAGTCTTTAAAGGTGGTT
GACCAGGCATTTGTCAGAGTTAAGAAAGAGAGGTAGGACATCCTTTTCCAGGCA
GAGGGCATTGTGTGCACACACGTATAGAAGCAGGCAGCCCACCCTCATGCTTTC
CAGGAAGCAAATGTGGCTCAGGTGTAAAGTGCCCGGTTGATGAAGGGAGTTAGC
GGAGGGAGTATAAGGATGTACTGTCTGCCCCCTTAGGACACCTGCAGAGGATTA
AGGTGGCTGTTTCTCCCTGGAGGTGGAGTGGGTGGGTCACTGCACAGGAGCCTA
TAGTTGTTGGTCTTTTAAACTCTTATTGGTGTAACCAGCCACGGAACTCTGAGGC
AAGGGGTTGGGGGTGGGAAGGGAAACAGAGAAAAGGCAAGTGAAACAGAAGG
GGAGGTGCAGTTTCAGAACCCAGCCAGCCTCTCTCTTGCTGCCTAGCCTCCTGCC
GGCCTCATCTTCGCCCAGCCAACCCCGCCTGGAGCCCTATGCCTCGACTGTGCCT
TCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGT
CTGAGTAGGTGTCATTCTATTCATCGATCACGAGACTAGCCTCGAGAAGCTTGAT
ATCGAATTCCACGGGGTTGGACGCGTCTTAATTAAGGATCCAAGGTCAGGAACA
GAGAAACAGGAGAATATGGGCCAAACAGGATATCTGTGGTAAGCAGTTCCTGCC
CCGGCTCAGGGCCAAGAACAGTTGGAACAGCAGAATATGGGCCAAACAGGATA
TCTGTGGTAAGCAGTTCCTGCCCCGGCTCAGGGCCAAGAACAGATGGTCCCCAG
ATGCGGTCCCGCCCTCAGCAGTTTCTAGAGAACCATCAGATGTTTCCAGGGTGCC
CCAAGGACCTGAAATGACCCTGTGCCTTATTTGAACTAACCAATCAGTTCGCTTC
TCGCTTCTGTTCGCGCGCTTCTGCTCCCCGAGCTCTATATAAGCAGAGCTCGTTTA
GTGAACCGTCAGATCGCCTGGAGACGCCATCCACGCTGTTTTGACCTCCATAGAA
GACACCGACTCTAGAGGATCGATCCCCCGGGCTGCAGGAATTCAAGCGAGAAGA
CAAGGGCAGAAAGCACCGCCACCATGCTGCTGCTGGTGACCTCTCTGCTGCTGT
GCGAGCTGCCACACCCAGCCTTCCTGCTGATCCCAGACATCGTGATGGCCCAGTC
CCACAAGTTCATGTCTACCAGCGTGGGCGACAGGGTGTCTATCACATGCAAGGC
CTCTCAGGATGTGAGCACCGTGGTGGCCTGGTACCAGCAGAAGCCAGGCCAGAG
CCCCAAGCGCCTGATCTATTCCGCCTCTTACCGGTATATCGGAGTGCCAGACAGA
TTCACCGGAAGCGGATCCGGCACAGATTTCACCTTTACAATCAGCTCCGTGCAGG
CCGAGGATCTGGCCGTGTACTATTGTCAGCAGCACTACTCCCCCCCTTATACCTT
TGGCGGCGGCACAAAGCTGGAGATCAAGCGGGGCTCCACCTCTGGCAGCGGCAA
GCCTGGCAGCGGAGAGGGCTCCACAAAGGGACAGGTGCAGCTGCAGCAGTCCG
GAGCAGAGCTGGCCCGGCCCGGCACCTCCGTGAAGCTGTCTTGCAAGGCCAGCG
GCTACACCTTCACAGACTATTGGATGCAGTGGGTGAAGCAGAGACCAGGACAGG
GACTGGAGTGGATCGGAACCATCTACCCTGGCGACGGCGATACAGGCTATGCCC
AGAAGTTTAAGGGCAAGGCCACCCTGACAGCCGACAAGAGCTCCAAGACAGTGT
ACATGCACCTGTCTAGCCTGGCCTCTGAGGATAGCGCCGTGTACTATTGTGCCCG
GGGCGACTACTATGGCAGCAACTCCCTGGATTATTGGGGCCAGGGCACCTCTGT
GACAGTGTCCTCTGAGAGCAAGTACGGACCACCTTGCCCACCATGTCCTGCACC
AGAGTTCCTGGGAGGACCTTCCGTGTTCCTGTTTCCTCCAAAGCCAAAGGACACC
CTGATGATCAGCCGGACCCCAGAGGTGACATGCGTGGTGGTGGACGTGAGCCAG
GAGGACCCCGAGGTGCAGTTCAACTGGTACGTGGATGGCGTGGAGGTGCACAAT
GCCAAGACCAAGCCAAGAGAGGAGCAGTTTAACTCCACCTATAGGGTGGTGTCT
GTGCTGACAGTGCTGCACCAGGACTGGCTGAACGGCAAGGAGTACAAGTGCAAG
GTGTCCAATAAGGGCCTGCCTTCCTCTATCGAGAAGACCATCTCTAAGGCAAAG
GGACAGCCAAGGGAGCCACAGGTGTATACACTGCCCCCTAGCCAGGAGGAGAT
GACCAAGAACCAGGTGTCCCTGACATGTCTGGTGAAGGGCTTTTACCCTTCTGAC
ATCGCCGTGGAGTGGGAGAGCAATGGCCAGCCAGAGAACAATTATAAGACCAC
ACCACCCGTGCTGGACTCTGATGGCAGCTTCTTTCTGTACAGCCGCCTGACCGTG
GATAAGTCCCGGTGGCAGGAGGGCAACGTGTTCTCCTGCTCTGTGATGCACGAG
GCCCTGCACAATCACTACACACAGAAGAGCCTGTCCCTGTCTCTGGGCAAGCGG
GGCAGAAAGAAGCTGCTGTACATCTTCAAGCAGCCCTTTATGAGGCCTGTGCAG
ACCACACAGGAGGAGGACGGCTGCAGCTGTCGGTTCCCCGAGGAGGAGGAGGG
CGGCTGCGAGCTGCGGGTGAAGTTTAGCAGATCCGCCGATGCACCAGCATATCA
GCAGGGACAGAATCAGCTGTACAACGAGCTGAATCTGGGCAGGCGCGAGGAGT
ACGACGTGCTGGATAAGAGGCGGGGCCGGGACCCCGAGATGGGAGGCAAGCCC
AGGCGCAAGAACCCTCAGGAGGGCCTGTATAATGAGCTGCAGAAGGACAAGAT
GGCCGAGGCCTACAGCGAGATCGGCATGAAGGGAGAGCGGAGAAGGGGCAAGG
GACACGATGGCCTGTATCAGGGCCTGTCCACCGCCACAAAGGACACCTACGATG
CACTGCACATGCAGGCCCTGCCACCTCGGTGAAAGTAACGCCCTCGACTGTGCCT
TCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGT
CTGAGTAGGTGTCATTCTATTCGCCAACTGCGAGTTCAGCCCGGTGTCCGGGGAC
AAACCCTGCTGCCGGCTCTCTAGGAGAGCCCAACTCTGTCTTGGCGTCAGTATCC
TGGTCCTGATCCTCGTCGTGGTGCTCGCGGTGGTCGTCCCGAGGTGGCGCCAGCA
GTGGAGCGGTCCGGGCACCACCAAGCGCTTTCCCGAGACCGTCCTGGCGCGATG
CGTCAAGTACACTGAAATTCATCCTGAGATGAGGTGGGTTGGCGACTAAGGCGC
ACCGGTGGGCACTGCGGGGACAGCAGGGCCCCGCGCGCAGGGAAGCCGCCCGG
ATCGCCCGGAACCGGGCATCTTCCGTGGCGGGTCAGCCGAGAGCCCGCCGGGTG
GTGCTGAGTAGGGAGTCCCGGGCTCGGGGCTCCGCGGGCCGCTTTCAGGAGCAG
CTGGCCTTGGCACCGAGCGTGCCCGCGGGAGGCGGGGGGGGGCGCTGCTCGGTG
GCTCTGCTGCGTAGCCGGTGAACACTTGGCACCGATGCCCGCCTTCTGGGCAAG
GTGCCCTGAGCCCAGCCCCTCGCCGGGCTGCAGCCCACCCTCGGCGCGCTCAGC
CCGCTTCACCGCTTCAGGGACGGAATGCGGCCGCAGGAACCCCTAGTGATGGAG
TTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCA
GCTGCCTGCAGGGGCGCCTGATGCGGTATTTTCTCCTTACGCATCTGTGCGGTAT
TTCACACCGCATACGTCAAAGCAACCATAGTACGCGCCCTGTAGCGGCGCATTA
AGCGCGGCGGGTGTGGTGGTTACGCGCAGCGTGACCGCTACACTTGCCAGCGCC
TTAGCGCCCGCTCCTTTCGCTTTCTTCCCTTCCTTTCTCGCCACGTTCGCCGGCTTT
CCCCGTCAAGCTCTAAATCGGGGGCTCCCTTTAGGGTTCCGATTTAGTGCTTTAC
GGCACCTCGACCCCAAAAAACTTGATTTGGGTGATGGTTCACGTAGTGGGCCATC
GCCCTGATAGACGGTTTTTCGCCCTTTGACGTTGGAGTCCACGTTCTTTAATAGTG
GACTCTTGTTCCAAACTGGAACAACACTCAACTCTATCTCGGGCTATTCTTTTGAT
TTATAAGGGATTTTGCCGATTTCGGTCTATTGGTTAAAAAATGAGCTGATTTAAC
AAAAATTTAACGCGAATTTTAACAAAATATTAACGTTTACAATTTTATGGTGCAC
TCTCAGTACAATCTGCTCTGATGCCGCATAGTTAAGCCAGCCCCGACACCCGCCA
ACACCCGCTGACGCGCCCTGACGGGCTTGTCTGCTCCCGGCATCCGCTTACAGAC
AAGCTGTGACCGTCTCCGGGAGCTGCATGTGTCAGAGGTTTTCACCGTCATCACC
GAAACGCGCGAGACGAAAGGGCCTCGTGATACGCCTATTTTTATAGGTTAATGT
CATGATAATAATGGTTTCTTAGACGTCAGGTGGCACTTTTCGGGGAAATGTGCGC
GGAACCCCTATTTGTTTATTTTTCTAAATACATTCAAATATGTATCCGCTCATGAG
ACAATAACCCTGATAAATGCTTCAATAATATTGAAAAAGGAAGAGTatgattgaacaag
atggattgcacgcaggttctccggccgcttgggtggagaggctattcggctatgactgggcacaacagacaatcggctgctctgatgc
cgccgtgttccggctgtcagcgcaggggcgcccggttctttttgtcaagaccgacctgtccggtgccctgaatgaactgcaggacga
ggcagcgcggctatcgtggctggccacgacgggcgttccttgcgcagclgigctcgacgtigtcactgaagcgggaagggactggc
tgctattgggcgaagtgccggggcaggatctcctgtcatctcaccttgctcctgccgagaaagtatccatcatggctgatgcaatgcgg
cggctgcatacgcttgatccggctacctgcccattcgaccaccaagegaaacatcgcatcgagcgagcacgtactcggatggaagc
cggtcttgtcgatcaggatgatctggacgaagagcatcaggggctcgcgccagccgaactgttcgccaggctcaaggcgcgcatgc
ccgacggcgaggatctcgtcgtgacccatggcgatgcctgcttgccgaatatcatggtggaaaatggccgcttttctggattcatcgac
tgtggccggctgggtgtggcggaccgctatcaggacatagcgttggctacccgtgatattgctgaagagcttggcggcgaatgggct
gaccgcttcctcgtgctttacggtatcgccgctcccgattcgcagcgcatcgccttctatcgccttcttgacgagttcttctgaCTGTC
AGACCAAGTTTACTCATATATACTTTAGATTGATTTAAAACTTCATTTTTAATTTA
AAAGGATCTAGGTGAAGATCCTTTTTGATAATCTCATGACCAAAATCCCTTAACG
TGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGC
TACCAGCGGTGGTTTGTTTGCCGGATCAAGAGCTACCAACTCTTTTTCCGAAGGT
AACTGGCTTCAGCAGAGCGCAGATACCAAATACTGTTCTTCTAGTGTAGCCGTAG
TTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGA
CTCAAGACGATAGTTACCGGATAAGGCGCAGCGGTCGGGCTGAACGGGGGGTTC
GTGCACACAGCCCAGCTTGGAGCGAACGACCTACACCGAACTGAGATACCTACA
GCGTGAGCTATGAGAAAGCGCCACGCTTCCCGAAGGGAGAAAGGCGGACAGGT
ATCCGGTAAGCGGCAGGGTCGGAACAGGAGAGCGCACGAGGGAGCTTCCAGGG
GGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGT
anti-CD38 ScFv v2 CD38CAR_V2_ISA-HLIG-41 ssAAV-BackBone_Kan (FIG. 13)
SEQ ID NO: 25
MLLLVTSLLLCELPHPAFLLIPQVQLQQSGAELARPGTSVKLSCKASGYTFTDYWMQ
WVKQRPGQGLEWIGTIYPGDGDTGYAQKFKGKATLTADKSSKTVYMHLSSLASEDS
AVYYCARGDYYGSNSLDYWGQGTSVTVSSGSTSGSGKPGSGEGSTKGDIVMAQSH
KFMSTSVGDRVSITCKASQDVSTVVAWYQQKPGQSPKRLIYSASYRYIGVPDRFTGS
GSGTDFTFTISSVQAEDLAVYYCQQHYSPPYTFGGGTKLEIKR
CD38CAR_V2_ISA-HLIG-41 ssAAV-BackBone_Kan
SEQ ID NO: 26
CCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCTGCGGCCGGCGCGCCGAAGTACAGAGAGGTTAA
GTAACTTGCCCCATGTCATCCAGCAAGAACTAAATTTGAACCCAGAGCTTAGCC
ACTGATGCCTCTTGAGAGAAGGAGTCAGACTTAAGTTGAGTCTTTAAAGGTGGTT
GACCAGGCATTTGTCAGAGTTAAGAAAGAGAGGTAGGACATCCTTTTCCAGGCA
GAGGGCATTGTGTGCACACACGTATAGAAGCAGGCAGCCCACCCTCATGCTTTC
CAGGAAGCAAATGTGGCTCAGGTGTAAAGTGCCCGGTTGATGAAGGGAGTTAGC
GGAGGGAGTATAAGGATGTACTGTCTGCCCCCTTAGGACACCTGCAGAGGATTA
AGGTGGCTGTTTCTCCCTGGAGGTGGAGTGGGTGGGTCACTGCACAGGAGCCTA
TAGTTGTTGGTCTTTTAAACTCTTATTGGTGTAACCAGCCACGGAACTCTGAGGC
AAGGGGTTGGGGGTGGGAAGGGAAACAGAGAAAAGGCAAGTGAAACAGAAGG
GGAGGTGCAGTTTCAGAACCCAGCCAGCCTCTCTCTTGCTGCCTAGCCTCCTGCC
GGCCTCATCTTCGCCCAGCCAACCCCGCCTGGAGCCCTATGCCTCGACTGTGCCT
TCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGT
CTGAGTAGGTGTCATTCTATTCATCGATCACGAGACTAGCCTCGAGAAGCTTGAT
ATCGAATTCCACGGGGTTGGACGCGTCTTAATTAAGGATCCAAGGTCAGGAACA
GAGAAACAGGAGAATATGGGCCAAACAGGATATCTGTGGTAAGCAGTTCCTGCC
CCGGCTCAGGGCCAAGAACAGTTGGAACAGCAGAATATGGGCCAAACAGGATA
TCTGTGGTAAGCAGTTCCTGCCCCGGCTCAGGGCCAAGAACAGATGGTCCCCAG
ATGCGGTCCCGCCCTCAGCAGTTTCTAGAGAACCATCAGATGTTTCCAGGGTGCC
CCAAGGACCTGAAATGACCCTGTGCCTTATTTGAACTAACCAATCAGTTCGCTTC
TCGCTTCTGTTCGCGCGCTTCTGCTCCCCGAGCTCTATATAAGCAGAGCTCGTTTA
GTGAACCGTCAGATCGCCTGGAGACGCCATCCACGCTGTTTTGACCTCCATAGAA
GACACCGACTCTAGAGGATCGATCCCCCGGGCTGCAGGAATTCAAGCGAGAAGA
CAAGGGCAGAAAGCACCGCCACCATGCTGCTGCTGGTGACCTCTCTGCTGCTGT
GCGAGCTGCCACACCCAGCCTTCCTGCTGATCCCACAGGTGCAGCTGCAGCAGT
CCGGAGCAGAGCTGGCCCGGCCCGGCACCTCCGTGAAGCTGTCTTGCAAGGCCA
GCGGCTACACCTTCACAGACTATTGGATGCAGTGGGTGAAGCAGAGACCAGGAC
AGGGACTGGAGTGGATCGGAACCATCTACCCTGGCGACGGCGATACAGGCTATG
CCCAGAAGTTTAAGGGCAAGGCCACCCTGACAGCCGACAAGAGCTCCAAGACA
GTGTACATGCACCTGTCTAGCCTGGCCTCTGAGGATAGCGCCGTGTACTATTGTG
CCCGGGGCGACTACTATGGCAGCAACTCCCTGGATTATTGGGGCCAGGGCACCT
CTGTGACAGTGTCCTCTGGCTCCACCTCTGGCAGCGGCAAGCCTGGCAGCGGAG
AGGGCTCCACAAAGGGAGACATCGTGATGGCCCAGTCCCACAAGTTCATGTCTA
CCAGCGTGGGCGACAGGGTGTCTATCACATGCAAGGCCTCTCAGGATGTGAGCA
CCGTGGTGGCCTGGTACCAGCAGAAGCCAGGCCAGAGCCCCAAGCGCCTGATCT
ATTCCGCCTCTTACCGGTATATCGGAGTGCCAGACAGATTCACCGGAAGCGGAT
CCGGCACAGATTTCACCTTTACAATCAGCTCCGTGCAGGCCGAGGATCTGGCCGT
GTACTATTGTCAGCAGCACTACTCCCCCCCTTATACCTTTGGCGGCGGCACAAAG
CTGGAGATCAAGCGGGAGAGCAAGTACGGACCACCTTGCCCACCATGTCCTGCA
CCAGAGTTCCTGGGAGGACCTTCCGTGTTCCTGTTTCCTCCAAAGCCAAAGGACA
CCCTGATGATCAGCCGGACCCCAGAGGTGACATGCGTGGTGGTGGACGTGAGCC
AGGAGGACCCCGAGGTGCAGTTCAACTGGTACGTGGATGGCGTGGAGGTGCACA
ATGCCAAGACCAAGCCAAGAGAGGAGCAGTTTAACTCCACCTATAGGGTGGTGT
CTGTGCTGACAGTGCTGCACCAGGACTGGCTGAACGGCAAGGAGTACAAGTGCA
AGGTGTCCAATAAGGGCCTGCCTTCCTCTATCGAGAAGACCATCTCTAAGGCAA
AGGGACAGCCAAGGGAGCCACAGGTGTATACACTGCCCCCTAGCCAGGAGGAG
ATGACCAAGAACCAGGTGTCCCTGACATGTCTGGTGAAGGGCTTTTACCCTTCTG
ACATCGCCGTGGAGTGGGAGAGCAATGGCCAGCCAGAGAACAATTATAAGACC
ACACCACCCGTGCTGGACTCTGATGGCAGCTTCTTTCTGTACAGCCGCCTGACCG
TGGATAAGTCCCGGTGGCAGGAGGGCAACGTGTTCTCCTGCTCTGTGATGCACG
AGGCCCTGCACAATCACTACACACAGAAGAGCCTGTCCCTGTCTCTGGGCAAGC
GGGGCAGAAAGAAGCTGCTGTACATCTTCAAGCAGCCCTTTATGAGGCCTGTGC
AGACCACACAGGAGGAGGACGGCTGCAGCTGTCGGTTCCCCGAGGAGGAGGAG
GGCGGCTGCGAGCTGCGGGTGAAGTTTAGCAGATCCGCCGATGCACCAGCATAT
CAGCAGGGACAGAATCAGCTGTACAACGAGCTGAATCTGGGCAGGCGCGAGGA
GTACGACGTGCTGGATAAGAGGCGGGGCCGGGACCCCGAGATGGGAGGCAAGC
CCAGGCGCAAGAACCCTCAGGAGGGCCTGTATAATGAGCTGCAGAAGGACAAG
ATGGCCGAGGCCTACAGCGAGATCGGCATGAAGGGAGAGCGGAGAAGGGGCAA
GGGACACGATGGCCTGTATCAGGGCCTGTCCACCGCCACAAAGGACACCTACGA
TGCACTGCACATGCAGGCCCTGCCACCTCGGTGAAAGTAACGCCCTCGACTGTG
CCTTCTAGTTGCCAGCCATCTGTTGTTTGCCCCTCCCCCGTGCCTTCCTTGACCCT
GGAAGGTGCCACTCCCACTGTCCTTTCCTAATAAAATGAGGAAATTGCATCGCAT
TGTCTGAGTAGGTGTCATTCTATTCGCCAACTGCGAGTTCAGCCCGGTGTCCGGG
GACAAACCCTGCTGCCGGCTCTCTAGGAGAGCCCAACTCTGTCTTGGCGTCAGTA
TCCTGGTCCTGATCCTCGTCGTGGTGCTCGCGGTGGTCGTCCCGAGGTGGCGCCA
GCAGTGGAGCGGTCCGGGCACCACCAAGCGCTTTCCCGAGACCGTCCTGGCGCG
ATGCGTCAAGTACACTGAAATTCATCCTGAGATGAGGTGGGTTGGCGACTAAGG
CGCACCGGTGGGCACTGCGGGGACAGCAGGGCCCCGCGCGCAGGGAAGCCGCC
CGGATCGCCCGGAACCGGGCATCTTCCGTGGCGGGTCAGCCGAGAGCCCGCCGG
GTGGTGCTGAGTAGGGAGTCCCGGGCTCGGGGCTCCGCGGGCCGCTTTCAGGAG
CAGCTGGCCTTGGCACCGAGCGTGCCCGCGGGAGGCGGGGGGGGGCGCTGCTCG
GTGGCTCTGCTGCGTAGCCGGTGAACACTTGGCACCGATGCCCGCCTTCTGGGCA
AGGTGCCCTGAGCCCAGCCCCTCGCCGGGCTGCAGCCCACCCTCGGCGCGCTCA
GCCCGCTTCACCGCTTCAGGGACGGAATGCGGCCGCAGGAACCCCTAGTGATGG
AGTTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCGCG
CAGCTGCCTGCAGGGGCGCCTGATGCGGTATTTTCTCCTTACGCATCTGTGCGGT
ATTTCACACCGCATACGTCAAAGCAACCATAGTACGCGCCCTGTAGCGGCGCAT
TAAGCGCGGCGGGTGTGGTGGTTACGCGCAGCGTGACCGCTACACTTGCCAGCG
CCTTAGCGCCCGCTCCTTTCGCTTTCTTCCCTTCCTTTCTCGCCACGTTCGCCGGC
TTTCCCCGTCAAGCTCTAAATCGGGGGCTCCCTTTAGGGTTCCGATTTAGTGCTTT
ACGGCACCTCGACCCCAAAAAACTTGATTTGGGTGATGGTTCACGTAGTGGGCC
ATCGCCCTGATAGACGGTTTTTCGCCCTTTGACGTTGGAGTCCACGTTCTTTAATA
GTGGACTCTTGTTCCAAACTGGAACAACACTCAACTCTATCTCGGGCTATTCTTTT
GATTTATAAGGGATTTTGCCGATTTCGGTCTATTGGTTAAAAAATGAGCTGATTT
AACAAAAATTTAACGCGAATTTTAACAAAATATTAACGTTTACAATTTTATGGTG
CACTCTCAGTACAATCTGCTCTGATGCCGCATAGTTAAGCCAGCCCCGACACCCG
CCAACACCCGCTGACGCGCCCTGACGGGCTTGTCTGCTCCCGGCATCCGCTTACA
GACAAGCTGTGACCGTCTCCGGGAGCTGCATGTGTCAGAGGTTTTCACCGTCATC
ACCGAAACGCGCGAGACGAAAGGGCCTCGTGATACGCCTATTTTTATAGGTTAA
TGTCATGATAATAATGGTTTCTTAGACGTCAGGTGGCACTTTTCGGGGAAATGTG
CGCGGAACCCCTATTTGTTTATTTTTCTAAATACATTCAAATATGTATCCGCTCAT
GAGACAATAACCCTGATAAATGCTTCAATAATATTGAAAAAGGAAGAGTatgattgaa
caagatggattgcacgcaggttctccggccgcttgggtggagaggctattcggctatgactgggcacaacagacaatcggctgctct
gatgccgccgtgttccggctgtcagcgcaggggcgcccggttctttttgtcaagaccgacctgtccggtgccctgaatgaactgcagg
acgaggcagcgcggctatcgtggctggccacgacgggcgttccttgcgcagctgtgctcgacgttgtcactgaagcgggaaggga
ctggctgctattgggcgaagtgccggggcaggatctcctgtcatctcaccttgctcctgccgagaaagtatccatcatggctgatgcaa
tgcggcggctgcatacgcttgatccggctacctgcccattcgaccaccaagcgaaacatcgcatcgagcgagcacgtactcggatgg
aagccggtcttgtcgatcaggatgatctggacgaagagcatcaggggctcgcgccagccgaactgttcgccaggctcaaggcgcgc
atgcccgacggcgaggatctcgtcgtgacccatggcgatgcctgcttgccgaatatcatggtggaaaatggccgcttttctggattcat
cgactgtggccggctgggtgtggcggaccgctatcaggacatagcgttggctacccgtgatattgctgaagagcttggcggcgaatg
ggctgaccgcttcctcgtgctttacggtatcgccgctcccgattcgcagcgcatcgccttctatcgccttcttgacgagttcttctgaCT
GTCAGACCAAGTTTACTCATATATACTTTAGATTGATTTAAAACTTCATTTTTAAT
TTAAAAGGATCTAGGTGAAGATCCTTTTTGATAATCTCATGACCAAAATCCCTTA
ACGTGAGTTTTCGTTCCACTGAGCGTCAGACCCCGTAGAAAAGATCAAAGGATC
TTCTTGAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTTTCCGA
AGGTAACTGGCTTCAGCAGAGCGCAGATACCAAATACTGTTCTTCTAGTGTAGCC
GTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGT
TGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACC
TACAGCGTGAGCTATGAGAAAGCGCCACGCTTCCCGAAGGGAGAAAGGCGGAC
AGGTATCCGGTAAGCGGCAGGGTCGGAACAGGAGAGCGCACGAGGGAGCTTCC
AGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGT
anti-CD38 ScFv v3 CD38CAR_V3_ISA-LHCD8-41 ssAAV-BackBone_Kan
SEQ ID NO: 27
MLLLVTSLLLCELPHPAFLLIPDIVMAQSHKFMSTSVGDRVSITCKASQDVSTVVAW
YQQKPGQSPKRLIYSASYRYIGVPDRFTGSGSGTDFTFTISSVQAEDLAVYYCQQHYS
PPYTFGGGTKLEIKRGSTSGSGKPGSGEGSTKGQVQLQQSGAELARPGTSVKLSCKA
SGYTFTDYWMQWVKQRPGQGLEWIGTIYPGDGDTGYAQKFKGKATLTADKSSKTV
YMHLSSLASEDSAVYYCARGDYYGSNSLDYWGQGTSVTVSS
CD38CAR_V3_ISA-LHCD8-41 ssAAV-BackBone_Kan
SEQ ID NO: 28
CCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCTGCGGCCGGCGCGCCGAAGTACAGAGAGGTTAA
GTAACTTGCCCCATGTCATCCAGCAAGAACTAAATTTGAACCCAGAGCTTAGCC
ACTGATGCCTCTTGAGAGAAGGAGTCAGACTTAAGTTGAGTCTTTAAAGGTGGTT
GACCAGGCATTTGTCAGAGTTAAGAAAGAGAGGTAGGACATCCTTTTCCAGGCA
GAGGGCATTGTGTGCACACACGTATAGAAGCAGGCAGCCCACCCTCATGCTTTC
CAGGAAGCAAATGTGGCTCAGGTGTAAAGTGCCCGGTTGATGAAGGGAGTTAGC
GGAGGGAGTATAAGGATGTACTGTCTGCCCCCTTAGGACACCTGCAGAGGATTA
AGGTGGCTGTTTCTCCCTGGAGGTGGAGTGGGTGGGTCACTGCACAGGAGCCTA
TAGTTGTTGGTCTTTTAAACTCTTATTGGTGTAACCAGCCACGGAACTCTGAGGC
AAGGGGTTGGGGGTGGGAAGGGAAACAGAGAAAAGGCAAGTGAAACAGAAGG
GGAGGTGCAGTTTCAGAACCCAGCCAGCCTCTCTCTTGCTGCCTAGCCTCCTGCC
GGCCTCATCTTCGCCCAGCCAACCCCGCCTGGAGCCCTATGCCTCGACTGTGCCT
TCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGT
CTGAGTAGGTGTCATTCTATTCATCGATCACGAGACTAGCCTCGAGAAGCTTGAT
ATCGAATTCCACGGGGTTGGACGCGTCTTAATTAAGGATCCAAGGTCAGGAACA
GAGAAACAGGAGAATATGGGCCAAACAGGATATCTGTGGTAAGCAGTTCCTGCC
CCGGCTCAGGGCCAAGAACAGTTGGAACAGCAGAATATGGGCCAAACAGGATA
TCTGTGGTAAGCAGTTCCTGCCCCGGCTCAGGGCCAAGAACAGATGGTCCCCAG
ATGCGGTCCCGCCCTCAGCAGTTTCTAGAGAACCATCAGATGTTTCCAGGGTGCC
CCAAGGACCTGAAATGACCCTGTGCCTTATTTGAACTAACCAATCAGTTCGCTTC
TCGCTTCTGTTCGCGCGCTTCTGCTCCCCGAGCTCTATATAAGCAGAGCTCGTTTA
GTGAACCGTCAGATCGCCTGGAGACGCCATCCACGCTGTTTTGACCTCCATAGAA
GACACCGACTCTAGAGGATCGATCCCCCGGGCTGCAGGAATTCAAGCGAGAAGA
CAAGGGCAGAAAGCACCGCCACCATGCTGCTGCTGGTGACCTCTCTGCTGCTGT
GCGAGCTGCCACACCCAGCCTTCCTGCTGATCCCAGACATCGTGATGGCCCAGTC
CCACAAGTTCATGTCTACCAGCGTGGGCGACAGGGTGTCTATCACATGCAAGGC
CTCTCAGGATGTGAGCACCGTGGTGGCCTGGTACCAGCAGAAGCCAGGCCAGAG
CCCCAAGCGCCTGATCTATTCCGCCTCTTACCGGTATATCGGAGTGCCAGACAGA
TTCACCGGAAGCGGATCCGGCACAGATTTCACCTTTACAATCAGCTCCGTGCAGG
CCGAGGATCTGGCCGTGTACTATTGTCAGCAGCACTACTCCCCCCCTTATACCTT
TGGCGGCGGCACAAAGCTGGAGATCAAGCGGGGCTCCACCTCTGGCAGCGGCAA
GCCTGGCAGCGGAGAGGGCTCCACAAAGGGACAGGTGCAGCTGCAGCAGTCCG
GAGCAGAGCTGGCCCGGCCCGGCACCTCCGTGAAGCTGTCTTGCAAGGCCAGCG
GCTACACCTTCACAGACTATTGGATGCAGTGGGTGAAGCAGAGACCAGGACAGG
GACTGGAGTGGATCGGAACCATCTACCCTGGCGACGGCGATACAGGCTATGCCC
AGAAGTTTAAGGGCAAGGCCACCCTGACAGCCGACAAGAGCTCCAAGACAGTGT
ACATGCACCTGTCTAGCCTGGCCTCTGAGGATAGCGCCGTGTACTATTGTGCCCG
GGGCGACTACTATGGCAGCAACTCCCTGGATTATTGGGGCCAGGGCACCTCTGT
GACAGTGTCCTCTACAACAACTCCAGCACCCCGCCCACCTACTCCCGCTCCTACA
ATCGCCTCTCAGCCCCTGTCCCTGCGCCCAGAAGCCTGTCGCCCCGCAGCAGGAG
GAGCAGTGCACACCAGGGGCCTGGACTTCGCCTGCGATATCTACATCTGGGCTC
CTCTGGCTGGAACTTGTGGCGTGCTGCTGCTGTCACTGGTCATTACCCTGTATTGT
AAGCGGGGCAGAAAGAAGCTGCTGTACATCTTCAAGCAGCCCTTTATGAGGCCT
GTGCAGACCACACAGGAGGAGGACGGCTGCAGCTGTCGGTTCCCCGAGGAGGA
GGAGGGCGGCTGCGAGCTGCGGGTGAAGTTTAGCAGATCCGCCGATGCACCAGC
ATATCAGCAGGGACAGAATCAGCTGTACAACGAGCTGAATCTGGGCAGGCGCGA
GGAGTACGACGTGCTGGATAAGAGGCGGGGCCGGGACCCCGAGATGGGAGGCA
AGCCCAGGCGCAAGAACCCTCAGGAGGGCCTGTATAATGAGCTGCAGAAGGAC
AAGATGGCCGAGGCCTACAGCGAGATCGGCATGAAGGGAGAGCGGAGAAGGGG
CAAGGGACACGATGGCCTGTATCAGGGCCTGTCCACCGCCACAAAGGACACCTA
CGATGCACTGCACATGCAGGCCCTGCCACCTCGGTGAAAGTAACGCCCTCGACT
GTGCCTTCTAGTTGCCAGCCATCTGTTGTTTGCCCCTCCCCCGTGCCTTCCTTGAC
CCTGGAAGGTGCCACTCCCACTGTCCTTTCCTAATAAAATGAGGAAATTGCATCG
CATTGTCTGAGTAGGTGTCATTCTATTCGCCAACTGCGAGTTCAGCCCGGTGTCC
GGGGACAAACCCTGCTGCCGGCTCTCTAGGAGAGCCCAACTCTGTCTTGGCGTC
AGTATCCTGGTCCTGATCCTCGTCGTGGTGCTCGCGGTGGTCGTCCCGAGGTGGC
GCCAGCAGTGGAGCGGTCCGGGCACCACCAAGCGCTTTCCCGAGACCGTCCTGG
CGCGATGCGTCAAGTACACTGAAATTCATCCTGAGATGAGGTGGGTTGGCGACT
AAGGCGCACCGGTGGGCACTGCGGGGACAGCAGGGCCCCGCGCGCAGGGAAGC
CGCCCGGATCGCCCGGAACCGGGCATCTTCCGTGGCGGGTCAGCCGAGAGCCCG
CCGGGTGGTGCTGAGTAGGGAGTCCCGGGCTCGGGGCTCCGCGGGCCGCTTTCA
GGAGCAGCTGGCCTTGGCACCGAGCGTGCCCGCGGGAGGCGGGGGGGGGCGCT
GCTCGGTGGCTCTGCTGCGTAGCCGGTGAACACTTGGCACCGATGCCCGCCTTCT
GGGCAAGGTGCCCTGAGCCCAGCCCCTCGCCGGGCTGCAGCCCACCCTCGGCGC
GCTCAGCCCGCTTCACCGCTTCAGGGACGGAATGCGGCCGCAGGAACCCCTAGT
GATGGAGTTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGA
GCGCGCAGCTGCCTGCAGGGGCGCCTGATGCGGTATTTTCTCCTTACGCATCTGT
GCGGTATTTCACACCGCATACGTCAAAGCAACCATAGTACGCGCCCTGTAGCGG
CGCATTAAGCGCGGCGGGTGTGGTGGTTACGCGCAGCGTGACCGCTACACTTGC
CAGCGCCTTAGCGCCCGCTCCTTTCGCTTTCTTCCCTTCCTTTCTCGCCACGTTCG
CCGGCTTTCCCCGTCAAGCTCTAAATCGGGGGCTCCCTTTAGGGTTCCGATTTAG
TGCTTTACGGCACCTCGACCCCAAAAAACTTGATTTGGGTGATGGTTCACGTAGT
GGGCCATCGCCCTGATAGACGGTTTTTCGCCCTTTGACGTTGGAGTCCACGTTCT
TTAATAGTGGACTCTTGTTCCAAACTGGAACAACACTCAACTCTATCTCGGGCTA
TTCTTTTGATTTATAAGGGATTTTGCCGATTTCGGTCTATTGGTTAAAAAATGAGC
TGATTTAACAAAAATTTAACGCGAATTTTAACAAAATATTAACGTTTACAATTTT
ATGGTGCACTCTCAGTACAATCTGCTCTGATGCCGCATAGTTAAGCCAGCCCCGA
CACCCGCCAACACCCGCTGACGCGCCCTGACGGGCTTGTCTGCTCCCGGCATCCG
CTTACAGACAAGCTGTGACCGTCTCCGGGAGCTGCATGTGTCAGAGGTTTTCACC
GTCATCACCGAAACGCGCGAGACGAAAGGGCCTCGTGATACGCCTATTTTTATA
GGTTAATGTCATGATAATAATGGTTTCTTAGACGTCAGGTGGCACTTTTCGGGGA
AATGTGCGCGGAACCCCTATTTGTTTATTTTTCTAAATACATTCAAATATGTATCC
GCTCATGAGACAATAACCCTGATAAATGCTTCAATAATATTGAAAAAGGAAGAG
Tatgattgaacaagatggattgcacgcaggttctccggccgcttgggtggagaggctattcggctatgactgggcacaacagacaat
cggctgctctgatgccgccgtgttccggctgtcagcgcaggggcgcccggttctttttgtcaagaccgacctgtccggtgccctgaatg
aactgcaggacgaggcagcgcggctatcgtggctggccacgacgggcgttccttgcgcagctgtgctcgacgttgtcactgaagcg
ggaagggactggctgctattgggcgaagtgccggggcaggatctcctgtcatctcaccttgctcctgccgagaaagtatccatcatgg
ctgatgcaatgcggcggctgcatacgcttgatccggctacctgcccattcgaccaccaagcgaaacatcgcatcgagcgagcacgta
ctcggatggaagccggtcttgtcgatcaggatgatctggacgaagagcatcaggggctcgcgccagccgaactgttcgccaggctc
aaggcgcgcatgcccgacggcgaggatctcgtcgtgacccatggcgatgcctgcttgccgaatatcatggtggaaaatggccgctttt
ctggattcatcgactgtggccggctgggtgtggcggaccgctatcaggacatagcgttggctacccgtgatattgctgaagagcttgg
cggcgaatgggctgaccgcttcctcgtgctttacggtatcgccgctcccgattcgcagcgcatcgccttctatcgccttcttgacgagtt
cttctgaCTGTCAGACCAAGTTTACTCATATATACTTTAGATTGATTTAAAACTTCATT
TTTAATTTAAAAGGATCTAGGTGAAGATCCTTTTTGATAATCTCATGACCAAAAT
CCCTTAACGTGAGTTTTCGTTCCACTGAGCGTCAGACCCCGTAGAAAAGATCAAA
GGATCTTCTTGAGATCCTTTTTTTCTGCGCGTAATCTGCTGCTTGCAAACAAAAA
AACCACCGCTACCAGCGGTGGTTTGTTTGCCGGATCAAGAGCTACCAACTCTTTT
TCCGAAGGTAACTGGCTTCAGCAGAGCGCAGATACCAAATACTGTTCTTCTAGTG
TAGCCGTAGTTAGGCCACCACTTCAAGAACTCTGTAGCACCGCCTACATACCTCG
CTCTGCTAATCCTGTTACCAGTGGCTGCTGCCAGTGGCGATAAGTCGTGTCTTAC
CGGGTTGGACTCAAGACGATAGTTACCGGATAAGGCGCAGCGGTCGGGCTGAAC
GGGGGGTTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTG
ACTTGAGCGTCGATTTTTGTGATGCTCGTCAGGGGGGCGGAGCCTATGGAAAAA
CGCCAGCAACGCGGCCTTTTTACGGTTCCTGGCCTTTTGCTGGCCTTTTGCTCACA
TGT
anti-CD38 ScFv v4 CD38CAR_V4_ISA-HLCD8-41 ssAAV-BackBone_Kan
SEQ ID NO: 29
MLLLVTSLLLCELPHPAFLLIPQVQLQQSGAELARPGTSVKLSCKASGYTFTDYWMQ
WVKQRPGQGLEWIGTIYPGDGDTGYAQKFKGKATLTADKSSKTVYMHLSSLASEDS
AVYYCARGDYYGSNSLDYWGQGTSVTVSSGSTSGSGKPGSGEGSTKGDIVMAQSH
KFMSTSVGDRVSITCKASQDVSTVVAWYQQKPGQSPKRLIYSASYRYIGVPDRFTGS
GSGTDFTFTISSVQAEDLAVYYCQQHYSPPYTFGGGTKLEIKR
CD38CAR_V4_ISA-HLCD8-41 ssAAV-BackBone_Kan
SEQ ID NO: 30
CCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCTGCGGCCGGCGCGCCGAAGTACAGAGAGGTTAA
GTAACTTGCCCCATGTCATCCAGCAAGAACTAAATTTGAACCCAGAGCTTAGCC
ACTGATGCCTCTTGAGAGAAGGAGTCAGACTTAAGTTGAGTCTTTAAAGGTGGTT
GACCAGGCATTTGTCAGAGTTAAGAAAGAGAGGTAGGACATCCTTTTCCAGGCA
GAGGGCATTGTGTGCACACACGTATAGAAGCAGGCAGCCCACCCTCATGCTTTC
CAGGAAGCAAATGTGGCTCAGGTGTAAAGTGCCCGGTTGATGAAGGGAGTTAGC
GGAGGGAGTATAAGGATGTACTGTCTGCCCCCTTAGGACACCTGCAGAGGATTA
AGGTGGCTGTTTCTCCCTGGAGGTGGAGTGGGTGGGTCACTGCACAGGAGCCTA
TAGTTGTTGGTCTTTTAAACTCTTATTGGTGTAACCAGCCACGGAACTCTGAGGC
AAGGGGTTGGGGGTGGGAAGGGAAACAGAGAAAAGGCAAGTGAAACAGAAGG
GGAGGTGCAGTTTCAGAACCCAGCCAGCCTCTCTCTTGCTGCCTAGCCTCCTGCC
GGCCTCATCTTCGCCCAGCCAACCCCGCCTGGAGCCCTATGCCTCGACTGTGCCT
TCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGT
CTGAGTAGGTGTCATTCTATTCATCGATCACGAGACTAGCCTCGAGAAGCTTGAT
ATCGAATTCCACGGGGTTGGACGCGTCTTAATTAAGGATCCAAGGTCAGGAACA
GAGAAACAGGAGAATATGGGCCAAACAGGATATCTGTGGTAAGCAGTTCCTGCC
CCGGCTCAGGGCCAAGAACAGTTGGAACAGCAGAATATGGGCCAAACAGGATA
TCTGTGGTAAGCAGTTCCTGCCCCGGCTCAGGGCCAAGAACAGATGGTCCCCAG
ATGCGGTCCCGCCCTCAGCAGTTTCTAGAGAACCATCAGATGTTTCCAGGGTGCC
CCAAGGACCTGAAATGACCCTGTGCCTTATTTGAACTAACCAATCAGTTCGCTTC
TCGCTTCTGTTCGCGCGCTTCTGCTCCCCGAGCTCTATATAAGCAGAGCTCGTTTA
GTGAACCGTCAGATCGCCTGGAGACGCCATCCACGCTGTTTTGACCTCCATAGAA
GACACCGACTCTAGAGGATCGATCCCCCGGGCTGCAGGAATTCAAGCGAGAAGA
CAAGGGCAGAAAGCACCGCCACCATGCTGCTGCTGGTGACCTCTCTGCTGCTGT
GCGAGCTGCCACACCCAGCCTTCCTGCTGATCCCACAGGTGCAGCTGCAGCAGT
CCGGAGCAGAGCTGGCCCGGCCCGGCACCTCCGTGAAGCTGTCTTGCAAGGCCA
GCGGCTACACCTTCACAGACTATTGGATGCAGTGGGTGAAGCAGAGACCAGGAC
AGGGACTGGAGTGGATCGGAACCATCTACCCTGGCGACGGCGATACAGGCTATG
CCCAGAAGTTTAAGGGCAAGGCCACCCTGACAGCCGACAAGAGCTCCAAGACA
GTGTACATGCACCTGTCTAGCCTGGCCTCTGAGGATAGCGCCGTGTACTATTGTG
CCCGGGGCGACTACTATGGCAGCAACTCCCTGGATTATTGGGGCCAGGGCACCT
CTGTGACAGTGTCCTCTGGCTCCACCTCTGGCAGCGGCAAGCCTGGCAGCGGAG
AGGGCTCCACAAAGGGAGACATCGTGATGGCCCAGTCCCACAAGTTCATGTCTA
CCAGCGTGGGCGACAGGGTGTCTATCACATGCAAGGCCTCTCAGGATGTGAGCA
CCGTGGTGGCCTGGTACCAGCAGAAGCCAGGCCAGAGCCCCAAGCGCCTGATCT
ATTCCGCCTCTTACCGGTATATCGGAGTGCCAGACAGATTCACCGGAAGCGGAT
CCGGCACAGATTTCACCTTTACAATCAGCTCCGTGCAGGCCGAGGATCTGGCCGT
GTACTATTGTCAGCAGCACTACTCCCCCCCTTATACCTTTGGCGGCGGCACAAAG
CTGGAGATCAAGCGGACAACAACTCCAGCACCCCGCCCACCTACTCCCGCTCCT
ACAATCGCCTCTCAGCCCCTGTCCCTGCGCCCAGAAGCCTGTCGCCCCGCAGCAG
GAGGAGCAGTGCACACCAGGGGCCTGGACTTCGCCTGCGATATCTACATCTGGG
CTCCTCTGGCTGGAACTTGTGGCGTGCTGCTGCTGTCACTGGTCATTACCCTGTAT
TGTAAGCGGGGCAGAAAGAAGCTGCTGTACATCTTCAAGCAGCCCTTTATGAGG
CCTGTGCAGACCACACAGGAGGAGGACGGCTGCAGCTGTCGGTTCCCCGAGGAG
GAGGAGGGCGGCTGCGAGCTGCGGGTGAAGTTTAGCAGATCCGCCGATGCACCA
GCATATCAGCAGGGACAGAATCAGCTGTACAACGAGCTGAATCTGGGCAGGCGC
GAGGAGTACGACGTGCTGGATAAGAGGCGGGGCCGGGACCCCGAGATGGGAGG
CAAGCCCAGGCGCAAGAACCCTCAGGAGGGCCTGTATAATGAGCTGCAGAAGG
ACAAGATGGCCGAGGCCTACAGCGAGATCGGCATGAAGGGAGAGCGGAGAAGG
GGCAAGGGACACGATGGCCTGTATCAGGGCCTGTCCACCGCCACAAAGGACACC
TACGATGCACTGCACATGCAGGCCCTGCCACCTCGGTGAAAGTAACGCCCTCGA
CTGTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGCAT
CGCATTGTCTGAGTAGGTGTCATTCTATTCGCCAACTGCGAGTTCAGCCCGGTGT
CCGGGGACAAACCCTGCTGCCGGCTCTCTAGGAGAGCCCAACTCTGTCTTGGCGT
CAGTATCCTGGTCCTGATCCTCGTCGTGGTGCTCGCGGTGGTCGTCCCGAGGTGG
CGCCAGCAGTGGAGCGGTCCGGGCACCACCAAGCGCTTTCCCGAGACCGTCCTG
GCGCGATGCGTCAAGTACACTGAAATTCATCCTGAGATGAGGTGGGTTGGCGAC
TAAGGCGCACCGGTGGGCACTGCGGGGACAGCAGGGCCCCGCGCGCAGGGAAG
CCGCCCGGATCGCCCGGAACCGGGCATCTTCCGTGGCGGGTCAGCCGAGAGCCC
GCCGGGTGGTGCTGAGTAGGGAGTCCCGGGCTCGGGGCTCCGCGGGCCGCTTTC
AGGAGCAGCTGGCCTTGGCACCGAGCGTGCCCGCGGGAGGCGGGGGGGGGCGC
TGCTCGGTGGCTCTGCTGCGTAGCCGGTGAACACTTGGCACCGATGCCCGCCTTC
TGGGCAAGGTGCCCTGAGCCCAGCCCCTCGCCGGGCTGCAGCCCACCCTCGGCG
CGCTCAGCCCGCTTCACCGCTTCAGGGACGGAATGCGGCCGCAGGAACCCCTAG
TGATGGAGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCGAGCG
AGCGCGCAGCTGCCTGCAGGGGCGCCTGATGCGGTATTTTCTCCTTACGCATCTG
TGCGGTATTTCACACCGCATACGTCAAAGCAACCATAGTACGCGCCCTGTAGCG
GCGCATTAAGCGCGGCGGGTGTGGTGGTTACGCGCAGCGTGACCGCTACACTTG
CCAGCGCCTTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTA
GTGCTTTACGGCACCTCGACCCCAAAAAACTTGATTTGGGTGATGGTTCACGTAG
TGGGCCATCGCCCTGATAGACGGTTTTTCGCCCTTTGACGTTGGAGTCCACGTTC
TTTAATAGTGGACTCTTGTTCCAAACTGGAACAACACTCAACTCTATCTCGGGCT
ATTCTTTTGATTTATAAGGGATTTTGCCGATTTCGGTCTATTGGTTAAAAAATGAG
CTGATTTAACAAAAATTTAACGCGAATTTTAACAAAATATTAACGTTTACAATTT
TATGGTGCACTCTCAGTACAATCTGCTCTGATGCCGCATAGTTAAGCCAGCCCCG
ACACCCGCCAACACCCGCTGACGCGCCCTGACGGGCTTGTCTGCTCCCGGCATCC
GCTTACAGACAAGCTGTGACCGTCTCCGGGAGCTGCATGTGTCAGAGGTTTTCAC
CGTCATCACCGAAACGCGCGAGACGAAAGGGCCTCGTGATACGCCTATTTTTAT
AGGTTAATGTCATGATAATAATGGTTTCTTAGACGTCAGGTGGCACTTTTCGGGG
AAATGTGCGCGGAACCCCTATTTGTTTATTTTTCTAAATACATTCAAATATGTATC
CGCTCATGAGACAATAACCCTGATAAATGCTTCAATAATATTGAAAAAGGAAGA
GTatgattgaacaagatggattgcacgcaggttctccggccgcttgggtggagaggctattcggctatgactgggcacaacagaca
atcggctgctctgatgccgccgtgttccggctgtcagcgcaggggcgcccggttctttttgtcaagaccgacctgtccggtgccctgaa
tgaactgcaggacgaggcagcgcggctatcgtggctggccacgacgggcgttccttgcgcagctgtgctcgacgttgtcactgaag
cgggaagggactggctgctattgggcgaagtgccggggcaggatctcctgtcatctcaccttgctcctgccgagaaagtatccatcat
ggctgatgcaatgcggcggctgcatacgcttgatccggctacctgcccattcgaccaccaagcgaaacategcatcgagcgagcac
gtactcggatggaagccggtcttgtcgatcaggatgatctggacgaagagcatcaggggctcgcgccagccgaactgttcgccagg
ctcaaggcgcgcatgcccgacggcgaggatctcgtcgtgacccatggcgatgcctgcttgccgaatatcatggtggaaaatggccg
cttttctggattcatcgactgtggccggctgggtgtggcggaccgctatcaggacatagcgttggctacccgtgatattgctgaagagct
tggcggcgaatgggctgaccgcttcctcgtgctttacggtatcgccgctcccgattcgcagcgcatcgccttctatcgccttcttgacga
gttcttctgaCTGTCAGACCAAGTTTACTCATATATACTTTAGATTGATTTAAAACTTCA
TTTTTAATTTAAAAGGATCTAGGTGAAGATCCTTTTTGATAATCTCATGACCAAA
ATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAA
AAAACCACCGCTACCAGCGGTGGTTTGTTTGCCGGATCAAGAGCTACCAACTCTT
TTTCCGAAGGTAACTGGCTTCAGCAGAGCGCAGATACCAAATACTGTTCTTCTAG
TGTAGCCGTAGTTAGGCCACCACTTCAAGAACTCTGTAGCACCGCCTACATACCT
CGCTCTGCTAATCCTGTTACCAGTGGCTGCTGCCAGTGGCGATAAGTCGTGTCTT
ACCGGGTTGGACTCAAGACGATAGTTACCGGATAAGGCGCAGCGGTCGGGCTGA
ACGGGGGGTTCGTGCACACAGCCCAGCTTGGAGCGAACGACCTACACCGAACTG
AGATACCTACAGCGTGAGCTATGAGAAAGCGCCACGCTTCCCGAAGGGAGAAA
GGCGGACAGGTATCCGGTAAGCGGCAGGGTCGGAACAGGAGAGCGCACGAGGG
AGCTTCCAGGGGGAAACGCCTGGTATCTTTATAGTCCTGTCGGGTTTCGCCACCT
CTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCA
CATGT
anti-CD38 ScFv v5 CD38CAR_V5_mISA-LHIG-41 ssAAV-BackBone_Kan
SEQ ID NO: 31
MLLLVTSLLLCELPHPAFLLIPDIVMTQSHLSMSTSLGDPVSITCKASQDVSTVVAWY
QQKPGQSPRRLIYSASYRYIGVPDRFTGSGAGTDFTFTISSVQAEDLAVYYCQQHYSP
PYTFGGGTKLEIKRGSTSGSGKPGSGEGSTKGQVQLVQSGAEVAKPGTSVKLSCKAS
GYTFTDYWMQWVKQRPGQGLEWIGTIYPGDGDTGYAQKFQGKATLTADKSSKTV
YMHLSSLASEDSAVYYCARGDYYGSNSLDYWGQGTSVTVSS
CD38CAR_V5_mISA-LHIG-41 ssAAV-BackBone_Kan
SEQ ID NO: 32
CCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCTGCGGCCGGCGCGCCGAAGTACAGAGAGGTTAA
GTAACTTGCCCCATGTCATCCAGCAAGAACTAAATTTGAACCCAGAGCTTAGCC
ACTGATGCCTCTTGAGAGAAGGAGTCAGACTTAAGTTGAGTCTTTAAAGGTGGTT
GACCAGGCATTTGTCAGAGTTAAGAAAGAGAGGTAGGACATCCTTTTCCAGGCA
GAGGGCATTGTGTGCACACACGTATAGAAGCAGGCAGCCCACCCTCATGCTTTC
CAGGAAGCAAATGTGGCTCAGGTGTAAAGTGCCCGGTTGATGAAGGGAGTTAGC
GGAGGGAGTATAAGGATGTACTGTCTGCCCCCTTAGGACACCTGCAGAGGATTA
AGGTGGCTGTTTCTCCCTGGAGGTGGAGTGGGTGGGTCACTGCACAGGAGCCTA
TAGTTGTTGGTCTTTTAAACTCTTATTGGTGTAACCAGCCACGGAACTCTGAGGC
AAGGGGTTGGGGGTGGGAAGGGAAACAGAGAAAAGGCAAGTGAAACAGAAGG
GGAGGTGCAGTTTCAGAACCCAGCCAGCCTCTCTCTTGCTGCCTAGCCTCCTGCC
GGCCTCATCTTCGCCCAGCCAACCCCGCCTGGAGCCCTATGCCTCGACTGTGCCT
TCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGT
CTGAGTAGGTGTCATTCTATTCATCGATCACGAGACTAGCCTCGAGAAGCTTGAT
ATCGAATTCCACGGGGTTGGACGCGTCTTAATTAAGGATCCAAGGTCAGGAACA
GAGAAACAGGAGAATATGGGCCAAACAGGATATCTGTGGTAAGCAGTTCCTGCC
CCGGCTCAGGGCCAAGAACAGTTGGAACAGCAGAATATGGGCCAAACAGGATA
TCTGTGGTAAGCAGTTCCTGCCCCGGCTCAGGGCCAAGAACAGATGGTCCCCAG
ATGCGGTCCCGCCCTCAGCAGTTTCTAGAGAACCATCAGATGTTTCCAGGGTGCC
CCAAGGACCTGAAATGACCCTGTGCCTTATTTGAACTAACCAATCAGTTCGCTTC
TCGCTTCTGTTCGCGCGCTTCTGCTCCCCGAGCTCTATATAAGCAGAGCTCGTTTA
GTGAACCGTCAGATCGCCTGGAGACGCCATCCACGCTGTTTTGACCTCCATAGAA
GACACCGACTCTAGAGGATCGATCCCCCGGGCTGCAGGAATTCAAGCGAGAAGA
CAAGGGCAGAAAGCACCGCCACCATGCTGCTGCTGGTGACCTCTCTGCTGCTGT
GCGAGCTGCCACACCCAGCCTTCCTGCTGATCCCAGACATCGTGATGACCCAGA
GCCACCTGTCTATGAGCACATCCCTGGGCGACCCCGTGAGCATCACCTGCAAGG
CCTCCCAGGACGTGAGCACAGTGGTGGCCTGGTACCAGCAGAAGCCAGGCCAGT
CTCCCCGGAGACTGATCTATTCTGCCAGCTACAGGTATATCGGCGTGCCAGACCG
CTTCACCGGAAGCGGAGCAGGCACAGATTTCACCTTTACAATCAGCTCCGTGCA
GGCCGAGGATCTGGCCGTGTACTATTGTCAGCAGCACTACTCCCCCCCTTATACC
TTTGGCGGCGGCACAAAGCTGGAGATCAAGCGGGGCTCCACCTCTGGCAGCGGC
AAGCCTGGCAGCGGAGAGGGCTCCACAAAGGGACAGGTGCAGCTGGTGCAGTC
CGGAGCAGAGGTGGCAAAGCCAGGCACCTCCGTGAAGCTGTCTTGCAAGGCCAG
CGGCTACACCTTCACAGACTATTGGATGCAGTGGGTGAAGCAGAGGCCAGGACA
GGGACTGGAGTGGATCGGCACCATCTACCCTGGCGACGGCGATACAGGCTATGC
CCAGAAGTTTCAGGGCAAGGCCACCCTGACAGCCGACAAGAGCTCCAAGACAGT
GTACATGCACCTGTCTAGCCTGGCCTCTGAGGATAGCGCCGTGTACTATTGTGCC
AGAGGCGACTACTATGGCAGCAACTCCCTGGATTATTGGGGCCAGGGCACCTCT
GTGACAGTGTCCTCTGAGAGCAAGTACGGACCACCTTGCCCACCATGTCCTGCAC
CAGAGTTCCTGGGAGGACCTTCCGTGTTCCTGTTTCCTCCAAAGCCAAAGGACAC
CCTGATGATCAGCCGGACCCCAGAGGTGACATGCGTGGTGGTGGACGTGAGCCA
GGAGGACCCCGAGGTGCAGTTCAACTGGTACGTGGATGGCGTGGAGGTGCACAA
TGCCAAGACCAAGCCAAGAGAGGAGCAGTTTAACTCCACCTATAGGGTGGTGTC
TGTGCTGACAGTGCTGCACCAGGACTGGCTGAACGGCAAGGAGTACAAGTGCAA
GGTGTCCAATAAGGGCCTGCCTTCCTCTATCGAGAAGACCATCTCTAAGGCAAA
GGGACAGCCAAGGGAGCCACAGGTGTATACACTGCCCCCTAGCCAGGAGGAGA
TGACCAAGAACCAGGTGTCCCTGACATGTCTGGTGAAGGGCTTTTACCCTTCTGA
CATCGCCGTGGAGTGGGAGAGCAATGGCCAGCCAGAGAACAATTATAAGACCA
CACCACCCGTGCTGGACTCTGATGGCAGCTTCTTTCTGTACAGCCGCCTGACCGT
GGATAAGTCCCGGTGGCAGGAGGGCAACGTGTTCTCCTGCTCTGTGATGCACGA
GGCCCTGCACAATCACTACACACAGAAGAGCCTGTCCCTGTCTCTGGGCAAGCG
GGGCAGAAAGAAGCTGCTGTACATCTTCAAGCAGCCCTTTATGAGGCCTGTGCA
GACCACACAGGAGGAGGACGGCTGCAGCTGTCGGTTCCCCGAGGAGGAGGAGG
GCGGCTGCGAGCTGCGGGTGAAGTTTAGCAGATCCGCCGATGCACCAGCATATC
AGCAGGGACAGAATCAGCTGTACAACGAGCTGAATCTGGGCAGGCGCGAGGAG
TACGACGTGCTGGATAAGAGGCGGGGCCGGGACCCCGAGATGGGAGGCAAGCC
CAGGCGCAAGAACCCTCAGGAGGGCCTGTATAATGAGCTGCAGAAGGACAAGA
TGGCCGAGGCCTACAGCGAGATCGGCATGAAGGGAGAGCGGAGAAGGGGCAAG
GGACACGATGGCCTGTATCAGGGCCTGTCCACCGCCACAAAGGACACCTACGAT
GCACTGCACATGCAGGCCCTGCCACCTCGGTGAAAGTAACGCCCTCGACTGTGC
CTTCTAGTTGCCAGCCATCTGTTGTTTGCCCCTCCCCCGTGCCTTCCTTGACCCTG
GAAGGTGCCACTCCCACTGTCCTTTCCTAATAAAATGAGGAAATTGCATCGCATT
GTCTGAGTAGGTGTCATTCTATTCGCCAACTGCGAGTTCAGCCCGGTGTCCGGGG
ACAAACCCTGCTGCCGGCTCTCTAGGAGAGCCCAACTCTGTCTTGGCGTCAGTAT
CCTGGTCCTGATCCTCGTCGTGGTGCTCGCGGTGGTCGTCCCGAGGTGGCGCCAG
CAGTGGAGCGGTCCGGGCACCACCAAGCGCTTTCCCGAGACCGTCCTGGCGCGA
TGCGTCAAGTACACTGAAATTCATCCTGAGATGAGGTGGGTTGGCGACTAAGGC
GCACCGGTGGGCACTGCGGGGACAGCAGGGCCCCGCGCGCAGGGAAGCCGCCC
GGATCGCCCGGAACCGGGCATCTTCCGTGGCGGGTCAGCCGAGAGCCCGCCGGG
TGGTGCTGAGTAGGGAGTCCCGGGCTCGGGGCTCCGCGGGCCGCTTTCAGGAGC
AGCTGGCCTTGGCACCGAGCGTGCCCGCGGGAGGCGGGGGGGGGCGCTGCTCGG
TGGCTCTGCTGCGTAGCCGGTGAACACTTGGCACCGATGCCCGCCTTCTGGGCAA
GGTGCCCTGAGCCCAGCCCCTCGCCGGGCTGCAGCCCACCCTCGGCGCGCTCAG
CCCGCTTCACCGCTTCAGGGACGGAATGCGGCCGCAGGAACCCCTAGTGATGGA
GTTGGCCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGCGCGC
AGCTGCCTGCAGGGGCGCCTGATGCGGTATTTTCTCCTTACGCATCTGTGCGGTA
TTTCACACCGCATACGTCAAAGCAACCATAGTACGCGCCCTGTAGCGGCGCATT
AAGCGCGGCGGGTGTGGTGGTTACGCGCAGCGTGACCGCTACACTTGCCAGCGC
CTTAGCGCCCGCTCCTTTCGCTTTCTTCCCTTCCTTTCTCGCCACGTTCGCCGGCTT
TCCCCGTCAAGCTCTAAATCGGGGGCTCCCTTTAGGGTTCCGATTTAGTGCTTTA
CGGCACCTCGACCCCAAAAAACTTGATTTGGGTGATGGTTCACGTAGTGGGCCAT
CGCCCTGATAGACGGTTTTTCGCCCTTTGACGTTGGAGTCCACGTTCTTTAATAGT
GGACTCTTGTTCCAAACTGGAACAACACTCAACTCTATCTCGGGCTATTCTTTTG
ATTTATAAGGGATTTTGCCGATTTCGGTCTATTGGTTAAAAAATGAGCTGATTTA
ACAAAAATTTAACGCGAATTTTAACAAAATATTAACGTTTACAATTTTATGGTGC
ACTCTCAGTACAATCTGCTCTGATGCCGCATAGTTAAGCCAGCCCCGACACCCGC
CAACACCCGCTGACGCGCCCTGACGGGCTTGTCTGCTCCCGGCATCCGCTTACAG
ACAAGCTGTGACCGTCTCCGGGAGCTGCATGTGTCAGAGGTTTTCACCGTCATCA
CCGAAACGCGCGAGACGAAAGGGCCTCGTGATACGCCTATTTTTATAGGTTAAT
GTCATGATAATAATGGTTTCTTAGACGTCAGGTGGCACTTTTCGGGGAAATGTGC
GCGGAACCCCTATTTGTTTATTTTTCTAAATACATTCAAATATGTATCCGCTCATG
AGACAATAACCCTGATAAATGCTTCAATAATATTGAAAAAGGAAGAGTatgattgaac
aagatggattgcacgcaggttctccggccgcttgggtggagaggctattcggctatgactgggcacaacagacaatcggctgctctg
atgccgccgtgttccggctgtcagcgcaggggcgcccggttctttttgtcaagaccgacctgtccggtgccctgaatgaactgcagga
cgaggcagcgcggctatcgtggctggccacgacgggcgttccttgcgcagctgtgctcgacgttgtcactgaagcgggaagggact
ggctgctattgggcgaagtgccggggcaggatctcctgtcatctcaccttgctcctgccgagaaagtatccatcatggctgatgcaatg
cggcggctgcatacgcttgatccggctacctgcccattcgaccaccaagcgaaacatcgcatcgagcgagcacgtactcggatgga
agccggtcttgtcgatcaggatgatctggacgaagagcatcaggggctcgcgccagccgaactgttcgccaggctcaaggcgcgca
tgcccgacggcgaggatctcgtcgtgacccatggcgatgcctgcttgccgaatatcatggtggaaaatggccgcttttctggattcatc
gactgtggccggctgggtgtggcggaccgctatcaggacatagcgttggctacccgtgatattgctgaagagcttggcggcgaatgg
gctgaccgcttcctcgtgctttacggtatcgccgctcccgattcgcagcgcatcgccttctatcgccttcttgacgagttcttctgaCTG
TCAGACCAAGTTTACTCATATATACTTTAGATTGATTTAAAACTTCATTTTTAATT
TAAAAGGATCTAGGTGAAGATCCTTTTTGATAATCTCATGACCAAAATCCCTTAA
CGTGAGTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTCCGAAG
GTAACTGGCTTCAGCAGAGCGCAGATACCAAATACTGTTCTTCTAGTGTAGCCGT
AGTTAGGCCACCACTTCAAGAACTCTGTAGCACCGCCTACATACCTCGCTCTGCT
AATCCTGTTACCAGTGGCTGCTGCCAGTGGCGATAAGTCGTGTCTTACCGGGTTG
GACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAG
GTATCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGACTTGA
GCGTCGATTTTTGTGATGCTCGTCAGGGGGGCGGAGCCTATGGAAAAACGCCAG
CAACGCGGCCTTTTTACGGTTCCTGGCCTTTTGCTGGCCTTTTGCTCACATGT

1. An engineered cell comprising a nucleic acid sequence encoding a chimeric antigen receptor (CAR) comprising a single-chain variable fragment (scFV) that specifically binds to a target molecule, wherein the nucleic acid sequence encoding the CAR is integrated into a CD38 gene locus, and wherein the engineered cell has a decreased expression of CD38 as compared with a reference control.

2. The engineered cell of claim 1, wherein the CD38 gene locus is at exon 1, exon 2, exon 3, exon 4, exon 5, exon 6, exon 7, and/or exon 8 of a CD38 gene.

3. (canceled)

4. The engineered cell of any claim 1, wherein the nucleic acid sequence encoding the CAR is integrated into the CD38 locus using a method comprising introducing into the cell a CRISPR/Cas endonuclease system with a CRISPR/Cas guide RNA, wherein the guide RNA targets the CD38 gene or a fragment thereof.

5. The engineered cell of claim 4, wherein the guide RNA comprises the sequence of CTGAACTCGCAGTTGGCCAT (SEQ IQ NO: 1) or a fragment thereof.

6. The engineered cell of claim 1, wherein the target molecule is a protein, a mutated protein, a polysaccharide, or a toxin.

7. (canceled)

8. The engineered cell of claim 6, wherein the protein or the mutated protein has an increased level in a subject or a cell in comparison to a reference control.

9. The engineered cell of claim 1, wherein the target molecule is CD33 or CD38.

10. The engineered cell of claim 1, wherein the target molecule is on a cell or a tumor cell.

11-12. (canceled)

13. The engineered cell of claim 1, wherein the CAR further comprises a transmembrane domain selected from a CD28 transmembrane domain, a CD3ζ transmembrane domain, or an NKG2D transmembrane domain.

14. The engineered cell of claim 1, wherein the CAR further comprises a co-stimulatory domain selected from a 2B4 domain, a CD28 co-stimulatory domain, a 4-1BB co-stimulatory domain, or any combination thereof.

15. The engineered cell of claim 1, wherein the CAR comprises the amino acid sequence as set forth in SEQ ID NO: 18, SEQ ID NO: 25, SEQ ID NO: 27, SEQ ID NO: 29, or SEQ ID NO: 31.

16. A pharmaceutical composition comprising an engineered cell and a pharmaceutically acceptable carrier, wherein the engineered cell comprises a nucleic acid sequence encoding a chimeric antigen receptor (CAR) comprising a single-chain variable fragment (scFV) that specifically binds to a target molecule, wherein the nucleic acid sequence encoding the CAR is integrated into a CD38 gene locus, and wherein the engineered cell has a decreased expression of CD38 as compared with a reference control.

17. The pharmaceutical composition of claim 16, further comprising an anti-CD38 inhibitor.

18. The pharmaceutical composition of claim 17, wherein the anti-CD38 inhibitor is an anti-CD38 antibody.

19-22. (canceled)

23. A method of making the engineered cell of claim 1, wherein the engineered cell comprising the chimeric antigen receptor (CAR) is created by:
a) obtaining a ribonucleoprotein (RNP) complex comprising a class 2 CRISPR/Cas endonuclease (Cas9) complexed with a corresponding CRISPR/Cas guide RNA and an AAV vector comprising a plasmid, nucleic acid, or construct comprising a polynucleotide sequence encoding a chimeric antigen receptor (CAR) polypeptide; wherein the polynucleotide sequence is flanked by homology arms; and wherein the homology arms are 800 bp in length or less; and
b) introducing the polynucleotide sequence encoding the CAR polypeptide and the RNP complex into a cell; wherein the polynucleotide sequence encoding the CAR polypeptide is introduced into the cell via infection with the AAV into the cell; wherein the RNP complex hybridizes to a target sequence within the genomic DNA of the cell and the cell's DNA repair enzymes insert the polynucleotide sequence encoding the CAR polypeptide into the host genome at the target sequence within the genomic DNA of the cell thereby creating the engineered cell.

24. The method of making an engineered cell of claim 23, wherein the left homology arm and right homology arm are the same length or are different lengths.

25. The method of making an engineered cell of claim 23,
wherein the homology arms are each 600 bp in length.

26-32. (canceled)

33. A method of treating a disease in a subject comprising administering to the subject a therapeutically effective amount of a engineered cell or a pharmaceutical composition comprising the engineered cell and a pharmaceutically acceptable carrier, wherein the engineered cell comprises a nucleic acid sequence encoding a chimeric antigen receptor (CAR) comprising a single-chain variable fragment (scFV) that specifically binds to a target molecule, wherein the nucleic acid sequence encoding the CAR is integrated into a CD38 gene locus, wherein the engineered cell has a decreased expression of CD38 as compared with a reference control.

34-37. (canceled)

38. The method of claim 33, further comprising administering to the subject a therapeutically effective amount of an agent to increase an expression of CD38.

39-42. (canceled)

43. The method of claim 33, wherein the disease comprises a minimal residual disease (MRD).

44-53. (canceled)


SEQUENCE LISTING (32 sequences)

SEQ ID NO: 1 - DNA, 20 bp, synthetic construct
  misc_feature 1..20: note = Description of sequence: gRNA
  source 1..20: mol_type = other DNA
ctgaactcgcagttggccat

SEQ ID NO: 2 - DNA, 600 bp, synthetic construct
  misc_feature 1..600: note = Description of sequence: Left CD38 Homology Arm
  source 1..600: mol_type = other DNA
gaagtacagagaggttaagtaacttgccccatgtcatccagcaagaactaaatttgaacccagagcttagccactgatgcctcttgagagaaggagtcagacttaagttgagtctttaaaggtggttgaccaggcatttgtcagagttaagaaagagaggtaggacatccttttccaggcagagggcattgtgtgcacacacgtatagaagcaggcagcccaccctcatgctttccaggaagcaaatgtggctcaggtgtaaagtgcccggttgatgaagggagttagcggagggagtataaggatgtactgtctgcccccttaggacacctgcagaggattaaggtggctgtttctccctggaggtggagtgggtgggtcactgcacaggagcctatagttgttggtcttttaaactcttattggtgtaaccagccacggaactctgaggcaaggggttgggggtgggaagggaaacagagaaaaggcaagtgaaacagaaggggaggtgcagtttcagaacccagccagcctctctcttgctgcctagcctcctgccggcctcatcttcgcccagccaaccccgcctggagccctatg

SEQ ID NO: 3 - DNA, 600 bp, synthetic construct
  misc_feature 1..600: note = Description of sequence: Right CD38 Homology Arm
  source 1..600: mol_type = other DNA
gccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttggcaccgagcgtgcccgcgggaggcgggggggggcgctgctcggtggctctgctgcgtagccggtgaacacttggcaccgatgcccgccttctgggcaaggtgccctgagcccagcccctcgccgggctgcagcccaccctcggcgcgctcagcccgcttcaccgcttcagggacggaat

SEQ ID NO: 4 - DNA, 147 bp, synthetic construct
  misc_feature 1..147: note = Description of sequence: BGH polyA terminator
  source 1..147: mol_type = other DNA
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc

SEQ ID NO: 5 - DNA, 20 bp, synthetic construct
  misc_feature 1..20: note = Description of sequence: crRNA
  source 1..20: mol_type = other DNA
ggggccactagggacaggat

SEQ ID NO: 6 - protein, 22 aa, synthetic construct
  REGION 1..22: note = Description of sequence: GM-CSRFRa signal peptide
MLLLVTSLLLCELPHPAFLLIP

SEQ ID NO: 7 - DNA, 207 bp, synthetic construct
  misc_feature 1..207: note = Description of sequence: CD28
  source 1..207: mol_type = other DNA
atgttttgggtgctggtggtggtgggaggcgtgctggcctgttattccctgctggtgaccgtggccttcatcatcttttgggtgcgctccaagcggagccggggcggacactctgactacatgaacatgaccccacggagacccggacctacaaggaagcactatcagccctacgcccctccacgggacttcgcagcatatcgcagc

SEQ ID NO: 8 - DNA, 339 bp, synthetic construct
  misc_feature 1..339: note = Description of sequence: CD3z
  source 1..339: mol_type = other DNA
cgggtgaagtttagcagatccgccgatgcaccagcatatcagcagggacagaatcagctgtacaacgagctgaatctgggcaggcgcgaggagtacgacgtgctggataagaggcggggccgggaccccgagatgggaggcaagcccaggcgcaagaaccctcaggagggcctgtataatgagctgcagaaggacaagatggccgaggcctacagcgagatcggcatgaagggagagcggagaaggggcaagggacacgatggcctgtatcagggcctgtccaccgccacaaaggacacctacgatgcactgcacatgcaggccctgccacctcggtga

SEQ ID NO: 9 - protein, 18 aa, synthetic construct
  REGION 1..18: note = Description of sequence: Whitlow linker
GSTSGSGKPGSGEGSTKG

SEQ ID NO: 10 - protein, 69 aa, synthetic construct
  REGION 1..69: note = Description of sequence: CD8 stalk region
TTTPAPRPPTPAPTIASQPLSLRPEACRPAAGGAVHTRGLDFACDIYIWAPLAGTCGVLLLSLVITLYC

SEQ ID NO: 11 - DNA, 93 bp, synthetic construct
  misc_feature 1..93: note = Description of sequence: NKG2D Transmembrane domain
  source 1..93: mol_type = other DNA
agcaacctgttcgtggcctcctggatcgccgtgatgatcatctttcgcatcggcatggccgtggccatcttctgctgtttctttttcccatcc

SEQ ID NO: 12 - DNA, 24 bp, synthetic construct
  misc_feature 1..24: note = Description of sequence: Linker
  source 1..24: mol_type = other DNA
ggaggctctggaggaggctccggc

SEQ ID NO: 13 - DNA, 360 bp, synthetic construct
  misc_feature 1..360: note = Description of sequence: 2B4
  source 1..360: mol_type = other DNA
tggcggagaaagcggaaggagaagcagagcgagacctcccctaaggagtttctgacaatctatgaggacgtgaaggatctgaagaccaggcgcaatcacgagcaggagcagaccttcccaggaggaggctctacaatctacagcatgatccagtcccagagcagcgccccaaccagccaggagccagcctatacactgtactctctgatccagcctagccggaagtctggcagccgcaagcggaaccactccccatctttcaattctaccatctatgaagtgatcggcaagagccagcctaaggcccagaacccagccagactgtccaggaaggagctggagaattttgacgtgtactct

SEQ ID NO: 14 - DNA, 24 bp, synthetic construct
  misc_feature 1..24: note = Description of sequence: Linker
  source 1..24: mol_type = other DNA
ggaggcagcggaggaggctctggc

SEQ ID NO: 15 - DNA, 342 bp, synthetic construct
  misc_feature 1..342: note = Description of sequence: CD3z
  source 1..342: mol_type = other DNA
cgcgtgaagttcagccggtccgccgatgccccagcctataagcagggccagaaccagctgtacaacgagctgaatctgggccggagagaggagtacgacgtgctggataagaggcggggccgggaccccgagatgggaggcaagccccggagaaagaaccctcaggagggcctgtataatgagctgcagaaggacaagatggccgaggcctactccgagatcggcatgaagggagagaggcgccggggcaagggacacgatggcctgtatcagggcctgagcaccgccacaaaggacacctacgatgccctgcacatgcaggccctgcctccacggtgatga

SEQ ID NO: 16 - DNA, 6349 bp, synthetic construct
  misc_feature 1..6349: note = Description of sequence: CD38CAR_V4_ISA-HLCD8-41__ssAAV-BackBone_KanAAAE46
  source 1..6349: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggcgcgccgaagtacagagaggttaagtaacttgccccatgtcatccagcaagaactaaatttgaacccagagcttagccactgatgcctcttgagagaaggagtcagacttaagttgagtctttaaaggtggttgaccaggcatttgtcagagttaagaaagagaggtaggacatccttttccaggcagagggcattgtgtgcacacacgtatagaagcaggcagcccaccctcatgctttccaggaagcaaatgtggctcaggtgtaaagtgcccggttgatgaagggagttagcggagggagtataaggatgtactgtctgcccccttaggacacctgcagaggattaaggtggctgtttctccctggaggtggagtgggtgggtcactgcacaggagcctatagttgttggtcttttaaactcttattggtgtaaccagccacggaactctgaggcaaggggttgggggtgggaagggaaacagagaaaaggcaagtgaaacagaaggggaggtgcagtttcagaacccagccagcctctctcttgctgcctagcctcctgccggcctcatcttcgcccagccaaccccgcctggagccctatgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcatcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccgccaccatgctgctgctggtgacctctctgctgctgtgcgagctgccacacccagccttcctgctgatcccacaggtgcagctgcagcagtccggagcagagctggcccggcccggcacctccgtgaagctgtcttgcaaggccagcggctacaccttcacagactattggatgcagtgggtgaagcagagaccaggacagggactggagtggatcggaaccatctaccctggcgacggcgatacaggctatgcccagaagtttaagggcaaggccaccctgacagccgacaagagctccaagacagtgtacatgcacctgtctagcctggcctctgaggatagcgccgtgtactattgtgcccggggcgactactatggcagcaactccctggattattggggccagggcacctctgtgacagtgtcctctggctccacctctggcagcggcaagcctggcagcggagagggctccacaaagggagacatcgtgatggcccagtcccacaagttcatgtctaccagcgtgggcgacagggtgtctatcacatgcaaggcctctcaggatgtgagcaccgtggtggcctggtaccagcagaagccaggccagagccccaagcgcctgatctattccgcctcttaccggtatatcggagtgccagacagattcaccggaagcggatccggcacagatttcacctttacaatcagctccgtgcaggccgaggatctggccgtgtactattgtcagcagcactactcccccccttatacctttggcggcggcacaaagctggagatcaagcggacaacaactccagcaccccgcccacctactcccgctcctacaatcgcctctcagcccctgtccctgcgcccagaagcctgtcgccccgcagcaggaggagcagtgcacaccaggggcctggacttcgcctgcgatatctacatctgggctcctctggctggaacttgtggcgtgctgctgctgtcactggtcattaccctgtattgtaagcggggcagaaagaagctgctgtacatcttcaagcagccctttatgaggcctgtgcagaccacacaggaggaggacggctgcagctgtcggttccccgaggaggaggagggcggctgcgagctgcgggtgaagtttagcagatccgccgatgcaccagcatatcagcagggacagaatcagctgtacaacgagctgaatctgggcaggcgcgaggagtacgacgtgctggataagaggcggggccgggaccccgagatgggaggcaagcccaggcgcaagaaccctcaggagggcctgtataatgagctgcagaaggacaagatggccgaggcctacagcgagatcggcatgaagggagagcggagaaggggcaagggacacgatggcctgtatcagggcctgtccaccgccacaaaggacacctacgatgcactgcacatgcaggccctgccacctcggtgaaagtaacgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttggcaccgagcgtgcccgcgggaggcgggggggggcgctgctcggtggctctgctgcgtagccggtgaacacttggcaccgatgcccgccttctgggcaaggtgccctgagcccagcccctcgccgggctgcagcccaccctcggcgcgctcagcccgcttcaccgcttcagggacggaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 17 - DNA, 540 bp, synthetic construct
  misc_feature 1..540: note = Description of sequence: MND promoter
  source 1..540: mol_type = other DNA
atcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcacc

SEQ ID NO: 18 - protein, 268 aa, synthetic construct
  REGION 1..268: note = Description of sequence: CD38CAR_V1_ISA-LHIG-41__ssAAV-BackBone_Kan
MLLLVTSLLLCELPHPAFLLIPDIVMAQSHKFMSTSVGDRVSITCKASQDVSTVVAWYQQKPGQSPKRLIYSASYRYIGVPDRFTGSGSGTDFTFTISSVQAEDLAVYYCQQHYSPPYTFGGGTKLEIKRGSTSGSGKPGSGEGSTKGQVQLQQSGAELARPGTSVKLSCKASGYTFTDYWMQWVKQRPGQGLEWIGTIYPGDGDTGYAQKFKGKATLTADKSSKTVYMHLSSLASEDSAVYYCARGDYYGSNSLDYWGQGTSVTVSS

SEQ ID NO: 19 - protein, 228 aa, synthetic construct
  REGION 1..228: note = Description of sequence: IgG Hinge CD4
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 42 aa, synthetic construct
  REGION 1..42: note = Description of sequence: 41BB-L
KRGRKKLLYIFKQPFMRPVQTTQEEDGCSCRFPEEEEGGCEL

SEQ ID NO: 21 - protein, 112 aa, synthetic construct
  REGION 1..112: note = Description of sequence: CD3z
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 747 bp, synthetic construct
  misc_feature 1..747: note = Description of sequence: Right Homology Arm
  source 1..747: mol_type = other DNA
gaagtacagagaggttaagtaacttgccccatgtcatccagcaagaactaaatttgaacccagagcttagccactgatgcctcttgagagaaggagtcagacttaagttgagtctttaaaggtggttgaccaggcatttgtcagagttaagaaagagaggtaggacatccttttccaggcagagggcattgtgtgcacacacgtatagaagcaggcagcccaccctcatgctttccaggaagcaaatgtggctcaggtgtaaagtgcccggttgatgaagggagttagcggagggagtataaggatgtactgtctgcccccttaggacacctgcagaggattaaggtggctgtttctccctggaggtggagtgggtgggtcactgcacaggagcctatagttgttggtcttttaaactcttattggtgtaaccagccacggaactctgaggcaaggggttgggggtgggaagggaaacagagaaaaggcaagtgaaacagaaggggaggtgcagtttcagaacccagccagcctctctcttgctgcctagcctcctgccggcctcatcttcgcccagccaaccccgcctggagccctatgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc

SEQ ID NO: 23 - DNA, 600 bp, synthetic construct; identical to SEQ ID NO: 3
  misc_feature 1..600: note = Description of sequence: Left Homology Arm
  source 1..600: mol_type = other DNA

SEQ ID NO: 24 - DNA, 6826 bp, synthetic construct
  misc_feature 1..6826: note = Description of sequence: CD38CAR_V1_ISA-LHIG-41__ssAAV-BackBone_Kan
  source 1..6826: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggcgcgccgaagtacagagaggttaagtaacttgccccatgtcatccagcaagaactaaatttgaacccagagcttagccactgatgcctcttgagagaaggagtcagacttaagttgagtctttaaaggtggttgaccaggcatttgtcagagttaagaaagagaggtaggacatccttttccaggcagagggcattgtgtgcacacacgtatagaagcaggcagcccaccctcatgctttccaggaagcaaatgtggctcaggtgtaaagtgcccggttgatgaagggagttagcggagggagtataaggatgtactgtctgcccccttaggacacctgcagaggattaaggtggctgtttctccctggaggtggagtgggtgggtcactgcacaggagcctatagttgttggtcttttaaactcttattggtgtaaccagccacggaactctgaggcaaggggttgggggtgggaagggaaacagagaaaaggcaagtgaaacagaaggggaggtgcagtttcagaacccagccagcctctctcttgctgcctagcctcctgccggcctcatcttcgcccagccaaccccgcctggagccctatgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcatcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccgccaccatgctgctgctggtgacctctctgctgctgtgcgagctgccacacccagccttcctgctgatcccagacatcgtgatggcccagtcccacaagttcatgtctaccagcgtgggcgacagggtgtctatcacatgcaaggcctctcaggatgtgagcaccgtggtggcctggtaccagcagaagccaggccagagccccaagcgcctgatctattccgcctcttaccggtatatcggagtgccagacagattcaccggaagcggatccggcacagatttcacctttacaatcagctccgtgcaggccgaggatctggccgtgtactattgtcagcagcactactcccccccttatacctttggcggcggcacaaagctggagatcaagcggggctccacctctggcagcggcaagcctggcagcggagagggctccacaaagggacaggtgcagctgcagcagtccggagcagagctggcccggcccggcacctccgtgaagctgtcttgcaaggccagcggctacaccttcacagactattggatgcagtgggtgaagcagagaccaggacagggactggagtggatcggaaccatctaccctggcgacggcgatacaggctatgcccagaagtttaagggcaaggccaccctgacagccgacaagagctccaagacagtgtacatgcacctgtctagcctggcctctgaggatagcgccgtgtactattgtgcccggggcgactactatggcagcaactccctggattattggggccagggcacctctgtgacagtgtcctctgagagcaagtacggaccaccttgcccaccatgtcctgcaccagagttcctgggaggaccttccgtgttcctgtttcctccaaagccaaaggacaccctgatgatcagccggaccccagaggtgacatgcgtggtggtggacgtgagccaggaggaccccgaggtgcagttcaactggtacgtggatggcgtggaggtgcacaatgccaagaccaagccaagagaggagcagtttaactccacctatagggtggtgtctgtgctgacagtgctgcaccaggactggctgaacggcaaggagtacaagtgcaaggtgtccaataagggcctgccttcctctatcgagaagaccatctctaaggcaaagggacagccaagggagccacaggtgtatacactgccccctagccaggaggagatgaccaagaaccaggtgtccctgacatgtctggtgaagggcttttacccttctgacatcgccgtggagtgggagagcaatggccagccagagaacaattataagaccacaccacccgtgctggactctgatggcagcttctttctgtacagccgcctgaccgtggataagtcccggtggcaggagggcaacgtgttctcctgctctgtgatgcacgaggccctgcacaatcactacacacagaagagcctgtccctgtctctgggcaagcggggcagaaagaagctgctgtacatcttcaagcagccctttatgaggcctgtgcagaccacacaggaggaggacggctgcagctgtcggttccccgaggaggaggagggcggctgcgagctgcgggtgaagtttagcagatccgccgatgcaccagcatatcagcagggacagaatcagctgtacaacgagctgaatctgggcaggcgcgaggagtacgacgtgctggataagaggcggggccgggaccccgagatgggaggcaagcccaggcgcaagaaccctcaggagggcctgtataatgagctgcagaaggacaagatggccgaggcctacagcgagatcggcatgaagggagagcggagaaggggcaagggacacgatggcctgtatcagggcctgtccaccgccacaaaggacacctacgatgcactgcacatgcaggccctgccacctcggtgaaagtaacgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttggcaccgagcgtgcccgcgggaggcgggggggggcgctgctcggtggctctgctgcgtagccggtgaacacttggcaccgatgcccgccttctgggcaaggtgccctgagcccagcccctcgccgggctgcagcccaccctcggcgcgctcagcccgcttcaccgcttcagggacggaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 25 - protein, 268 aa, synthetic construct
  REGION 1..268: note = Description of sequence: CD38CAR_V2_ISA-HLIG-41__ssAAV-BackBone_Kan
MLLLVTSLLLCELPHPAFLLIPQVQLQQSGAELARPGTSVKLSCKASGYTFTDYWMQWVKQRPGQGLEWIGTIYPGDGDTGYAQKFKGKATLTADKSSKTVYMHLSSLASEDSAVYYCARGDYYGSNSLDYWGQGTSVTVSSGSTSGSGKPGSGEGSTKGDIVMAQSHKFMSTSVGDRVSITCKASQDVSTVVAWYQQKPGQSPKRLIYSASYRYIGVPDRFTGSGSGTDFTFTISSVQAEDLAVYYCQQHYSPPYTFGGGTKLEIKR

SEQ ID NO: 26 - DNA, 6826 bp, synthetic construct
  misc_feature 1..6826: note = Description of sequence: CD38CAR_V2_ISA-HLIG-41__ssAAV-BackBone_Kan
  source 1..6826: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggcgcgccgaagtacagagaggttaagtaacttgccccatgtcatccagcaagaactaaatttgaacccagagcttagccactgatgcctcttgagagaaggagtcagacttaagttgagtctttaaaggtggttgaccaggcatttgtcagagttaagaaagagaggtaggacatccttttccaggcagagggcattgtgtgcacacacgtatagaagcaggcagcccaccctcatgctttccaggaagcaaatgtggctcaggtgtaaagtgcccggttgatgaagggagttagcggagggagtataaggatgtactgtctgcccccttaggacacctgcagaggattaaggtggctgtttctccctggaggtggagtgggtgggtcactgcacaggagcctatagttgttggtcttttaaactcttattggtgtaaccagccacggaactctgaggcaaggggttgggggtgggaagggaaacagagaaaaggcaagtgaaacagaaggggaggtgcagtttcagaacccagccagcctctctcttgctgcctagcctcctgccggcctcatcttcgcccagccaaccccgcctggagccctatgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcatcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccgccaccatgctgctgctggtgacctctctgctgctgtgcgagctgccacacccagccttcctgctgatcccacaggtgcagctgcagcagtccggagcagagctggcccggcccggcacctccgtgaagctgtcttgcaaggccagcggctacaccttcacagactattggatgcagtgggtgaagcagagaccaggacagggactggagtggatcggaaccatctaccctggcgacggcgatacaggctatgcccagaagtttaagggcaaggccaccctgacagccgacaagagctccaagacagtgtacatgcacctgtctagcctggcctctgaggatagcgccgtgtactattgtgcccggggcgactactatggcagcaactccctggattattggggccagggcacctctgtgacagtgtcctctggctccacctctggcagcggcaagcctggcagcggagagggctccacaaagggagacatcgtgatggcccagtcccacaagttcatgtctaccagcgtgggcgacagggtgtctatcacatgcaaggcctctcaggatgtgagcaccgtggtggcctggtaccagcagaagccaggccagagccccaagcgcctgatctattccgcctcttaccggtatatcggagtgccagacagattcaccggaagcggatccggcacagatttcacctttacaatcagctccgtgcaggccgaggatctggccgtgtactattgtcagcagcactactcccccccttatacctttggcggcggcacaaagctggagatcaagcgggagagcaagtacggaccaccttgcccaccatgtcctgcaccagagttcctgggaggaccttccgtgttcctgtttcctccaaagccaaaggacaccctgatgatcagccggaccccagaggtgacatgcgtggtggtggacgtgagccaggaggaccccgaggtgcagttcaactggtacgtggatggcgtggaggtgcacaatgccaagaccaagccaagagaggagcagtttaactccacctatagggtggtgtctgtgctgacagtgctgcaccaggactggctgaacggcaaggagtacaagtgcaaggtgtccaataagggcctgccttcctctatcgagaagaccatctctaaggcaaagggacagccaagggagccacaggtgtatacactgccccctagccaggaggagatgaccaagaaccaggtgtccctgacatgtctggtgaagggcttttacccttctgacatcgccgtggagtgggagagcaatggccagccagagaacaattataagaccacaccacccgtgctggactctgatggcagcttctttctgtacagccgcctgaccgtggataagtcccggtggcaggagggcaacgtgttctcctgctctgtgatgcacgaggccctgcacaatcactacacacagaagagcctgtccctgtctctgggcaagcggggcagaaagaagctgctgtacatcttcaagcagccctttatgaggcctgtgcagaccacacaggaggaggacggctgcagctgtcggttccccgaggaggaggagggcggctgcgagctgcgggtgaagtttagcagatccgccgatgcaccagcatatcagcagggacagaatcagctgtacaacgagctgaatctgggcaggcgcgaggagtacgacgtgctggataagaggcggggccgggaccccgagatgggaggcaagcccaggcgcaagaaccctcaggagggcctgtataatgagctgcagaaggacaagatggccgaggcctacagcgagatcggcatgaagggagagcggagaaggggcaagggacacgatggcctgtatcagggcctgtccaccgccacaaaggacacctacgatgcactgcacatgcaggccctgccacctcggtgaaagtaacgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttggcaccgagcgtgcccgcgggaggcgggggggggcgctgctcggtggctctgctgcgtagccggtgaacacttggcaccgatgcccgccttctgggcaaggtgccctgagcccagcccctcgccgggctgcagcccaccctcggcgcgctcagcccgcttcaccgcttcagggacggaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 27 - protein, 268 aa, synthetic construct; identical to SEQ ID NO: 18
  REGION 1..268: note = Description of sequence: CD38CAR_V3_ISA-LHCD8-41__ssAAV-BackBone_Kan

SEQ ID NO: 28 - DNA, 6349 bp, synthetic construct
  misc_feature 1..6349: note = Description of sequence: CD38CAR_V3_ISA-LHCD8-41__ssAAV-BackBone_Kan
  source 1..6349: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggcgcgccgaagtacagagaggttaagtaacttgccccatgtcatccagcaagaactaaatttgaacccagagcttagccactgatgcctcttgagagaaggagtcagacttaagttgagtctttaaaggtggttgaccaggcatttgtcagagttaagaaagagaggtaggacatccttttccaggcagagggcattgtgtgcacacacgtatagaagcaggcagcccaccctcatgctttccaggaagcaaatgtggctcaggtgtaaagtgcccggttgatgaagggagttagcggagggagtataaggatgtactgtctgcccccttaggacacctgcagaggattaaggtggctgtttctccctggaggtggagtgggtgggtcactgcacaggagcctatagttgttggtcttttaaactcttattggtgtaaccagccacggaactctgaggcaaggggttgggggtgggaagggaaacagagaaaaggcaagtgaaacagaaggggaggtgcagtttcagaacccagccagcctctctcttgctgcctagcctcctgccggcctcatcttcgcccagccaaccccgcctggagccctatgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcatcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccgccaccatgctgctgctggtgacctctctgctgctgtgcgagctgccacacccagccttcctgctgatcccagacatcgtgatggcccagtcccacaagttcatgtctaccagcgtgggcgacagggtgtctatcacatgcaaggcctctcaggatgtgagcaccgtggtggcctggtaccagcagaagccaggccagagccccaagcgcctgatctattccgcctcttaccggtatatcggagtgccagacagattcaccggaagcggatccggcacagatttcacctttacaatcagctccgtgcaggccgaggatctggccgtgtactattgtcagcagcactactcccccccttatacctttggcggcggcacaaagctggagatcaagcggggctccacctctggcagcggcaagcctggcagcggagagggctccacaaagggacaggtgcagctgcagcagtccggagcagagctggcccggcccggcacctccgtgaagctgtcttgcaaggccagcggctacaccttcacagactattggatgcagtgggtgaagcagagaccaggacagggactggagtggatcggaaccatctaccctggcgacggcgatacaggctatgcccagaagtttaagggcaaggccaccctgacagccgacaagagctccaagacagtgtacatgcacctgtctagcctggcctctgaggatagcgccgtgtactattgtgcccggggcgactactatggcagcaactccctggattattggggccagggcacctctgtgacagtgtcctctacaacaactccagcaccccgcccacctactcccgctcctacaatcgcctctcagcccctgtccctgcgcccagaagcctgtcgccccgcagcaggaggagcagtgcacaccaggggcctggacttcgcctgcgatatctacatctgggctcctctggctggaacttgtggcgtgctgctgctgtcactggtcattaccctgtattgtaagcggggcagaaagaagctgctgtacatcttcaagcagccctttatgaggcctgtgcagaccacacaggaggaggacggctgcagctgtcggttccccgaggaggaggagggcggctgcgagctgcgggtgaagtttagcagatccgccgatgcaccagcatatcagcagggacagaatcagctgtacaacgagctgaatctgggcaggcgcgaggagtacgacgtgctggataagaggcggggccgggaccccgagatgggaggcaagcccaggcgcaagaaccctcaggagggcctgtataatgagctgcagaaggacaagatggccgaggcctacagcgagatcggcatgaagggagagcggagaaggggcaagggacacgatggcctgtatcagggcctgtccaccgccacaaaggacacctacgatgcactgcacatgcaggccctgccacctcggtgaaagtaacgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttggcaccgagcgtgcccgcgggaggcgggggggggcgctgctcggtggctctgctgcgtagccggtgaacacttggcaccgatgcccgccttctgggcaaggtgccctgagcccagcccctcgccgggctgcagcccaccctcggcgcgctcagcccgcttcaccgcttcagggacggaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 29 - protein, 268 aa, synthetic construct; identical to SEQ ID NO: 25
  REGION 1..268: note = Description of sequence: CD38CAR_V4_ISA-HLCD8-41__ssAAV-BackBone_Kan

SEQ ID NO: 30 - DNA, 6349 bp, synthetic construct; identical to SEQ ID NO: 16
  misc_feature 1..6349: note = Description of sequence: CD38CAR_V4_ISA-HLCD8-41__ssAAV-BackBone_Kan
  source 1..6349: mol_type = other DNA

SEQ ID NO: 31 - protein, 268 aa, synthetic construct
  REGION 1..268: note = Description of sequence: CD38CAR_V5_mISA-LHIG-41__ssAAV-BackBone_Kan
MLLLVTSLLLCELPHPAFLLIPDIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRGSTSGSGKPGSGEGSTKGQVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSS

SEQ ID NO: 32 - DNA, 6826 bp, synthetic construct
  misc_feature 1..6826: note = Description of sequence: CD38CAR_V5_mISA-LHIG-41__ssAAV-BackBone_Kan
  source 1..6826: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggcgcgccgaagtacagagaggttaagtaacttgccccatgtcatccagcaagaactaaatttgaacccagagcttagccactgatgcctcttgagagaaggagtcagacttaagttgagtctttaaaggtggttgaccaggcatttgtcagagttaagaaagagaggtaggacatccttttccaggcagagggcattgtgtgcacacacgtatagaagcaggcagcccaccctcatgctttccaggaagcaaatgtggctcaggtgtaaagtgcccggttgatgaagggagttagcggagggagtataaggatgtactgtctgcccccttaggacacctgcagaggattaaggtggctgtttctccctggaggtggagtgggtgggtcactgcacaggagcctatagttgttggtcttttaaactcttattggtgtaaccagccacggaactctgaggcaaggggttgggggtgggaagggaaacagagaaaaggcaagtgaaacagaaggggaggtgcagtttcagaacccagccagcctctctcttgctgcctagcctcctgccggcctcatcttcgcccagccaaccccgcctggagccctatgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcatcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccgccaccatgctgctgctggtgacctctctgctgctgtgcgagctgccacacccagccttcctgctgatcccagacatcgtgatgacccagagccacctgtctatgagcacatccctgggcgaccccgtgagcatcacctgcaaggcctcccaggacgtgagcacagtggtggcctggtaccagcagaagccaggccagtctccccggagactgatctattctgccagctacaggtatatcggcgtgccagaccgcttcaccggaagcggagcaggcacagatttcacctttacaatcagctccgtgcaggccgaggatctggccgtgtactattgtcagcagcactactcccccccttatacctttggcggcggcacaaagctggagatcaagcggggctccacctctggcagcggcaagcctggcagcggagagggctccacaaagggacaggtgcagctggtgcagtccggagcagaggtggcaaagccaggcacctccgtgaagctgtcttgcaaggccagcggctacaccttcacagactattggatgcagtgggtgaagcagaggccaggacagggactggagtggatcggcaccatctaccctggcgacggcgatacaggctatgcccagaagtttcagggcaaggccaccctgacagccgacaagagctccaagacagtgtacatgcacctgtctagcctggcctctgaggatagcgccgtgtactattgtgccagaggcgactactatggcagcaactccctggattattggggccagggcacctctgtgacagtgtcctctgagagcaagtacggaccaccttgcccaccatgtcctgcaccagagttcctgggaggaccttccgtgttcctgtttcctccaaagccaaaggacaccctgatgatcagccggaccccagaggtgacatgcgtggtggtggacgtgagccaggaggaccccgaggtgcagttcaactggtacgtggatggcgtggaggtgcacaatgccaagaccaagccaagagaggagcagtttaactccacctatagggtggtgtctgtgctgacagtgctgcaccaggactggctgaacggcaaggagtacaagtgcaaggtgtccaataagggcctgccttcctctatcgagaagaccatctctaaggcaaagggacagccaagggagccacaggtgtatacactgccccctagccaggaggagatgaccaagaaccaggtgtccctgacatgtctggtgaagggcttttacccttctgacatcgccgtggagtgggagagcaatggccagccagagaacaattataagaccacaccacccgtgctggactctgatggcagcttctttctgtacagccgcctgaccgtggataagtcccggtggcaggagggcaacgtgttctcctgctctgtgatgcacgaggccctgcacaatcactacacacagaagagcctgtccctgtctctgggcaagcggggcagaaagaagctgctgtacatcttcaagcagccctttatgaggcctgtgcagaccacacaggaggaggacggctgcagctgtcggttccccgaggaggaggagggcggctgcgagctgcgggtgaagtttagcagatccgccgatgcaccagcatatcagcagggacagaatcagctgtacaacgagctgaatctgggcaggcgcgaggagtacgacgtgctggataagaggcggggccgggaccccgagatgggaggcaagcccaggcgcaagaaccctcaggagggcctgtataatgagctgcagaaggacaagatggccgaggcctacagcgagatcggcatgaagggagagcggagaaggggcaagggacacgatggcctgtatcagggcctgtccaccgccacaaaggacacctacgatgcactgcacatgcaggccctgccacctcggtgaaagtaacgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttggcaccgagcgtgcccgcgggaggcgggggggggcgctgctcggtggctctgctgcgtagccggtgaacacttggcaccgatgcccgccttctgggcaaggtgccctgagcccagcccctcgccgggctgcagcccaccctcggcgcgctcagcccgcttcaccgcttcagggacggaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt